SEQUENCE LISTING (27 sequences)

SEQ ID NO: 1 - protein, 14 aa, Artificial
  heavy chain CDR3H, <VEGF>ranibizumab
YPYYYGTSHWYFDV

SEQ ID NO: 2 - protein, 17 aa, Artificial
  heavy chain CDR2H, <VEGF>ranibizumab
WINTYTGEPTYAADFKR

SEQ ID NO: 3 - protein, 5 aa, Artificial
  heavy chain CDR1H, <VEGF>ranibizumab
HYGMN

SEQ ID NO: 4 - protein, 9 aa, Artificial
  light chain CDR3L,  <VEGF>ranibizumab
QQYSTVPWT

SEQ ID NO: 5 - protein, 7 aa, Artificial
  light chain CDR2L,  <VEGF>ranibizumab
FTSSLHS

SEQ ID NO: 6 - protein, 11 aa, Artificial
  light chain CDR1L,  <VEGF>ranibizumab
SASQDISNYLN

SEQ ID NO: 7 - protein, 123 aa, Artificial [sequence omitted]

SEQ ID NO: 8 - protein, 107 aa, Artificial [sequence omitted]

SEQ ID NO: 9 - protein, 20 aa, Artificial
  heavy chain CDR3H, <ANG-2> Ang2i_LC10 variant
SPNPYYYDSSGYYYPGAFDI

SEQ ID NO: 10 - protein, 17 aa, Artificial
  heavy chain CDR2H, <ANG-2> Ang2i_LC10 variant
WINPNSGGTNYAQKFQG

SEQ ID NO: 11 - protein, 5 aa, Artificial
  heavy chain CDR1H, <ANG-2> Ang2i_LC10 variant
GYYMH

SEQ ID NO: 12 - protein, 11 aa, Artificial
  light chain CDR3L, <ANG-2> Ang2i_LC10 variant
QVWDSSSDHWV

SEQ ID NO: 13 - protein, 7 aa, Artificial
  light chain CDR2L, <ANG-2> Ang2i_LC10 variant
DDSDRPS

SEQ ID NO: 14 - protein, 11 aa, Artificial
  light chain CDR1L, <ANG-2> Ang2i_LC10 variant
GGNNIGSKSVH

SEQ ID NO: 15 - protein, 129 aa, Artificial
  heavy chain variable domain VH, <ANG-2> Ang2i_LC10 variant
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSS

SEQ ID NO: 16 - protein, 110 aa, Artificial
  light chain variable domain VL, <ANG-2> Ang2i_LC10 variant
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVLSS

SEQ ID NO: 17 - protein, 451 aa, Artificial
  Heavy chain 1 of  <VEGF-ANG-2> CrossMAb IgG1 with AAA mutations and P329G LALA mutations (VEGFang2-0016)
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNAYTQKSLSLSP

SEQ ID NO: 18 - protein, 461 aa, Artificial
  Heavy chain 2 of  <VEGF-ANG-2> CrossMAb IgG1 with AAA mutations and P329G LALA mutations (VEGFang2-0016)
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSP

SEQ ID NO: 19 - protein, 214 aa, Artificial
  Light chain 1 of  <VEGF-ANG-2> CrossMAb IgG1 with AAA mutations and P329G LALA mutations (VEGFang2-0016)
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 213 aa, Artificial
  Light chain 2 of  <VEGF-ANG-2> CrossMAb IgG1 with AAA mutations and P329G LALA mutations (VEGFang2-0016)
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 21 - protein, 107 aa, Homo sapiens [sequence omitted]

SEQ ID NO: 22 - protein, 105 aa, homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 24 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 25 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 26 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 27 - protein, 1124 aa, Homo sapiens
MDSLASLVLCGVSLLLSGTVEGAMDLILINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIPRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPIFPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATITQYQLKGLEPETAYQVDIFAENNIGSSNPAFSHELVTLPESQAPADLGGGKMLLIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

(19) United States
(12) Patent Application Publication
(10) Pub. No.: US 2022/0162296 A1
(43) Pub. Date: May 26, 2022

(54) PERSONALIZED TREATMENT OF OPHTHALMOLOGIC DISEASES

(71) Applicants: Hoffmann-La Roche Inc., Little Falls, NJ (US); Genentech, Inc., South San Francisco, CA (US)

(72) Inventors: Hugh Lin, Burlingame, CA (US); Aaron Osborne, San Mateo, CA (US); David Andrew Silverman, London (GB); Robert James Weikert, Basel (CH); Jeffery R. Willis, San Francisco, CA (US)

(73) Assignees: Hoffmann-La Roche Inc., Little Falls, NJ (US); Genentech, Inc., South San Francisco, CA (US)

(21) Appl. No.: 17/665,144

(22) Filed: Feb. 4, 2022

Related U.S. Application Data
(63) Continuation of application No. PCT/EP2020/072088, filed on Aug. 6, 2020.
(60) Provisional application No. 62/883,499, filed on Aug. 6, 2019.

(51) Int. Cl. C07K 16/22; C07K 16/46; A61P 27/02

(52) U.S. Cl. CPC C07K 16/22; A61K 2039/545; A61P 27/02; C07K 16/468

(57) ABSTRACT

The current invention relates to antibodies which bind to VEGF and ANG2 for use in the treatment of ocular vascular diseases such as neovascular AMD (nAMD) (also known as choroidal neovascularization [CNV] secondary to age-related macular degeneration [AMD] or wet AMD), diabetic retinopathy in particular diabetic macular edema (DME) or macular edema secondary to retinal vein occlusion (RVO).

### CROSS REFERENCE TO RELATED APPLICATIONS

This application is a Continuation of International Application No. PCT/EP2020/072088, filed Aug. 6, 2020 claiming priority to U.S. Provisional Application No. 62/883,499 filed Aug. 6, 2019 which are both incorporated herein by reference in their entirety.

SEQUENCE LISTING

This application contains a Sequence Listing which has been submitted electronically in ASCII format and is hereby incorporated by reference in its entirety. Said ASCII copy, created on Jan. 24, 2022, is named Sequence_Listing.txt and is 44,568 bytes in size.

The current invention relates to antibodies, which bind to VEGF and ANG2 for use in the treatment of ocular vascular diseases such as neovascular AMD (nAMD) (also known as choroidal neovascularization [CNV] secondary to age-related macular degeneration [AMD] or wet AMD), diabetic retinopathy in particular diabetic macular edema (DME) or macular edema secondary to retinal vein occlusion (RVO).

### BACKGROUND OF THE INVENTION

Ocular vascular diseases such as neovascular AMD (nAMD) (also known as choroidal neovascularization [CNV] secondary to age-related macular degeneration [AMD] or wet AMD), diabetic retinopathy in particular diabetic macular edema (DME) are severe diseases leading to often to visual loss and blindness.

Neovascular age-related macular degeneration (nAMD) (also known as choroidal neovascularization [CNV] secondary to age-related macular degeneration [AMD] or wet AMD) is a form of advanced AMD that causes rapid and severe visual loss and remains a leading cause of visual impairment in the elderly (Bourne et al. Lancet Glob Health 2013; 1:e339-49; Wong et al. Lancet Glob Health 2014; 2:e106-16). Several biochemical and biological processes, such as angiogenesis, inflammation, and oxidative stress, are known to play a role in the pathogenesis of nAMD, which is characterized by the abnormal proliferation of choroidal capillaries that penetrate Bruch's membrane and migrate to or through the retinal pigment epithelium. CNV leaks fluid, lipids, and blood into the outer retina causing severe, irreversible loss of central vision if left untreated.

Prior to anti-vascular endothelial growth factor (anti-VEGF) agents, laser photocoagulation therapy and photodynamic therapy with verteporfin were the standard of care and were shown to stabilize vision. Although such treatments remain a therapeutic option for selected patients, the treatment of nAMD has been markedly improved by the introduction of biological molecules that target an important factor in pathological angiogenesis, VEGF-A (Brown et al. N Engl J Med 2006; 355:1432-44; Rosenfeld et al. N Engl J Med 2006; 355:1419-31; Heier et al. Ophthalmology 2012; 119:2537-48). The impressive benefit of anti-VEGF therapies and their ability to restore vision has been widely recognized since the first approval of Lucentis® (ranibizumab) in 2006 (American Academy of Ophthalmology 2015). A key challenge with currently available anti-VEGF treatments is the requirement for frequent and long-term administration to maintain vision gains (Heier et al. Ophthalmology 2012; 119:2537-48; the Comparison of Age-Related Macular Degeneration Treatment Trials [CATT] Research Group 2016 Ophthalmology 2016; 123:1751-61). Real-world data suggest that many patients with nAMD do not receive treatment at the optimal frequency, and this under-treatment in clinical practice is associated with lower visual acuity (VA) gains compared with those observed in controlled clinical trials (Cohen et al. Retina 2013; 33:474-81; Finger et al. Acta Ophthalmol 2013; 91:540-6; Holz et al. Br J Ophthalmol 2015; 99:220-6; Rao et al. Ophthalmology 2018; 125:522-28). Under-treatment of nAMD in clinical practice reflects the burden of frequent therapy on patients, caregivers, and the healthcare system (Gohil et al. PLoS One 2015; 10:e0129361; Prenner et al. Am J Ophthalmol 2015; 160:725-31; Varano et al. Clin Ophthalmol 2015; 9:2243-50; CATT Research Group et al. Ophthalmology 2016; 123:1751-61; Vukicevic et al. Eye 2016; 30: 413-21).

Diabetic macular edema (DME), a complication of diabetic retinopathy (DR), can develop at any stage of the underlying disease of retinal microvasculature (Fong et al. Diabetes Care 2004; 27:2540-53). DME occurs with increasing frequency as the underlying DR worsens (Henricsson et al. Acta Ophthalmol. Scand. 1999: 77: 218-223; Johnson Am J Ophthalmol 2009; 147:11-21) from non-proliferative DR (NPDR) to proliferative DR (PDR). DME is the most common cause of moderate and severe visual impairment in patients with DR (Ciulla et al. Diabetes Care 2003; 26:2653-64; Davidson et al. Endocrine 2007; 32:107-16; Leasher et al. Diabetes Care 2016; 39:1643-9), and if left untreated can lead to a loss of 10 or more letters in visual acuity (VA) within 2 years in approximately 50% of patients (Ferris and Patz Sury Ophthamol 1984; 28 Supp1:452-61; Diabetes Care 2003; 26:2653-64et al. 2003). DME affects approximately 14% of patients with diabetes and can be found in patients with both Type 1 and Type 2 diabetes (Girach and Lund-Andersen Int J Clin Practice 2007; 61:88-97). In 2013, the worldwide population of people with diabetes was approximately 382 million, and it is estimated to grow to 592 million by 2035 (International Diabetes Federation 2013). With advances in imaging technology, DME is now often diagnosed by optical coherence tomography (OCT) rather than the traditional Early Treatment Diabetic

Retinopathy Study (ETDRS) ophthalmoscopy-based criteria. On a molecular level, DME is a result of a vascular endothelial growth factor-A (VEGF-A)-mediated increase in vessel permeability and loss of pericytes, consequent to hypoxia-mediated release of pro-angiogenic, hyperpermeability, and pro-inflammatory mediators (Antonetti et al. Semin Ophthalmol 1999; 14:240-8). VEGF also upregulates a homeostatic factor, angiopoietin-2 (Ang-2), which acts as an antagonist of the Tie2 receptor tyrosine kinase on endothelial cells, counteracting vessel stabilization maintained through Ang-1-dependent Tie2 activation. Therefore, Ang-2 acts as a vascular destabilization factor, rendering the vasculature more elastic and amenable to endothelial barrier breakdown and sprouting. The excess of Ang-2 and VEGF in the retinal tissues promotes vessel destabilization, vascular leakage, and neovascularization. Ang-2 is also involved in inflammatory pathways such as lymphocyte recruitment. In summary, both VEGF-A and Ang-2 are recognized as key factors mediating diabetic eye disease pathogenesis (Aiello et al. N Engl J Med 1994; 331:1480-7; Davis et al. Cell 1996; 87:1161-9; Maisonpierre et al. Science 1997; 277:55-60; Gardner et al. Sury Ophthalmol 2002; 47(Suppl 2):5253-62; Joussen et al. Am J Path 2002; 160:501-9; Fiedler et al. J Biol Chem 2003; 278:1721-7).

Although macular laser used to be the standard of care (SOC) for treatment of DME, the development of anti-VEGF pharmacotherapy in the past 10 years has led to dramatic improvements in visual outcomes for patients with DME. Currently available anti-VEGF therapies for DME include ranibizumab and aflibercept. Other available approved options for the treatment of DME include periocular or intravitreal (IVT) steroids and steroid implants.

Despite the strong efficacy achieved with anti-VEGF therapies in DME, a significant proportion of patients do not experience clinically meaningful improvements in vision in the real world. Frequent IVT administration is required to achieve, and in some cases, to maintain the observed early benefits of DME treatment over a long period of time. The current SOC for administration of anti-VEGF injections requires patients to undergo frequent clinical examinations and IVT injections. This imposes a significant burden on patients, caregivers, treating physicians, and the healthcare system.

Large Phase III trials of anti-VEGF agents in DME demonstrated that after the first year of treatment, the number of injections needed for maintenance of vision gains can be decreased (Diabetic Retinopathy Clinical Research Network et al. Ophthalmology 2010:117:1064-77. Epub: 28 Apr. 2010; Schmidt-Erfurth et al. Ophthalmology 2014; 121:193-201; Elman et al. Ophthalmology 2015; 122:375-81). However, to achieve optimal outcomes in the absence of validated predictive biomarkers of treatment frequency, the standard anti-VEGF approach in DME still relies on frequent monitoring visits and places a substantial burden on patients and healthcare providers. In addition, anti-VEGF monotherapy does not fully address other pathways, including inflammation and pericyte destabilization, that contribute to worsening of diabetic eye disease.

New treatments that target additional pathways and that lead to reduced burden of IVT injections are needed to address high unmet medical need in DME.

### SUMMARY OF THE INVENTION

According to one aspect of the present invention, methods, uses, bispecific antibodies (for use), medicaments or pharmaceutical formulations are provided for the treatment of patients suffering from an ocular vascular disease selected from neovascular AMD (nAMD) and diabetic macular edema (DME), the method comprising administering to the patient an effective amount of a bispecific antibody which binds to human vascular endothelial growth factor (VEGF) and to human angiopoietin-2 (ANG-2) with personalized treatment interval (PTI) regimen wherein the treatment of patients suffering from an ocular vascular disease selected from nAMD and DME includes a dosing schedule that extends the administration interval in stable absence of disease, or shortens the interval if there is disease activity. In such a way patients are optimally treated ensuring improvement and/or maintenance of their visual acuity and at the same time reducing unnecessary treatment burden.

According to another aspect of the present invention, methods, uses, bispecific antibodies (for use), medicaments or pharmaceutical formulations are provided for the treatment of patients suffering from particular neovascular AMD (nAMD) (also called wet AMD (wAMD)), the method comprising administering to the patient an effective amount of a bispecific antibody which binds to human vascular endothelial growth factor (VEGF) and to human angiopoietin-2 (ANG-2) with personalized treatment interval (PTI) regimen wherein the treatment of patients suffering from nAMD includes a dosing schedule that extends the administration interval in stable absence of disease, or shortens the interval if there is disease activity. In such a way patients are optimally treated ensuring improvement and/or maintenance of their visual acuity and at the same time reducing unnecessary treatment burden.

According to one aspect of the present invention, methods, uses, bispecific antibodies (for use), medicaments or pharmaceutical formulations are provided for the treatment of patients suffering from the method comprising administering to the patient an effective amount of a bispecific antibody which binds to human vascular endothelial growth factor (VEGF) and to human angiopoietin-2 (ANG-2), wherein the treatment of patients suffering from AMD includes following treatment initiation a dosing schedule that extends the administration interval in stable absence of disease, or shortens the interval if there is disease activity.
One embodiment is such method, use, bispecific antibody (for use), medicament or pharmaceutical formulation for the treatment of patients suffering from neovascular AMD (nAMD) the method comprising administering to the patient an effective amount of a bispecific antibody which binds to human vascular endothelial growth factor (VEGF) and to human angiopoietin-2 (ANG-2) with a personalized treatment interval, wherein
a) patients are treated first 4 times with the bispecific VEGF/ANG2 antibody at an every 4 weeks (Q4W) dosing interval;
b) at Weeks 20 and 24 the disease activity is assessed wherein the disease activity is determined if one of the following criteria are met:
   i) increase of >50 μm in central subfield thickness (CST) compared with the average CST value over the previous two scheduled visits which are Weeks 12 and 16 for the Week 20 assessment, and Weeks 16 and 20 for the Week 24 assessment, or
   ii) increase >75 μm in CST compared with the lowest CST value recorded at either of the previous two scheduled visits;
   iii) decrease >5 letters in best-corrected visual acuity (BCVA) compared with average BCVA value over the previous two scheduled visits, owing to nAMD disease activity,
   iv) decrease 10 letters in BCVA compared with the highest BCVA value recorded at either of the previous two scheduled visits, owing to nAMD disease activity, or
   v) presence of new macular hemorrhage, owing to nAMD activity
c) then patients
   i) patients who meet the disease activity criteria at Week20 will be treated at an every 8 weeks (Q8W) dosing interval from week 20 onward (with the first Q8W dosing at Week20);
   ii) patients who meet the disease activity criteria at Week24 will be treated at an 12 weeks (Q12W) dosing interval from week 24 onward (with the first Q12W dosing at Week24); and
   iii) patients who do not meet disease activity criteria at Week20 and Week24 will be treated at an 16 weeks (Q16W) dosing interval from week 28 onward (with the first Q16W dosing at Week28).
In one embodiment the personalized treatment interval will be extended, reduced, or maintained after week 60 wherein the
   a) interval is extended by 4 weeks (to a maximum of Q16W) if all of the following criteria are met:
      i) stable CST compared with the average of the last 2 study drug dosing visits where stability is defined as a change of CST of less than 30 μm and no increase ≥50 μm in CST compared with the lowest on-study drug dosing visit measurement,
      ii) no decrease ≥5 letters in BCVA compared with the average from the last two study drug dosing visits, and no decrease ≥10 letters in BCVA compared with the highest on-study drug dosing visit measurement,
      iii) no new macular hemorrhage;
   b) interval is reduced (to a minimum Q8W) by 4 weeks if one of the following criteria is met,
      or
      is reduced to an 8-week interval if two or more of the following criteria are met or one criterion includes new macular hemorrhage:
      i) increase of ≥50 μm in CST compared with the average from the last two dosing visits or of ≥75 μm compared with the lowest dosing visit measurement,
      ii) decrease of ≥5 letters in BCVA compared with average of last two dosing visits or decrease ≥10 letters in BCVA compared with the highest dosing visit measurement,
      iii) new macular hemorrhage.
   According to another aspect of the present invention, methods, uses, bispecific antibodies (for use), medicaments or pharmaceutical formulations are provided for the treatment of patients suffering from diabetic retinopathy, in particular from diabetic macular edema (DME) the method comprising administering to the patient an effective amount of a bispecific antibody which binds to human vascular endothelial growth factor (VEGF) and to human angiopoietin-2 (ANG-2) with personalized treatment interval (PTI) regimen wherein the treatment of patients suffering from DME includes a dosing schedule that extends the administration interval in stable absence of disease, or shortens the interval if there is disease activity. In such a way patients are optimally treated ensuring improvement and/or maintenance of their visual acuity and at the same time reducing unnecessary treatment burden.
One embodiment is such method, use, bispecific antibody (for use), medicament or pharmaceutical formulation for the treatment of patients suffering from diabetic macular edema (DME) the method comprising administering to the patient an effective amount of a bispecific antibody which binds to human vascular endothelial growth factor (VEGF) and to human angiopoietin-2 (ANG-2) with a personalized treatment interval, wherein
a) patients are treated first with the bispecific VEGF/ANG2 antibody at an every 4 weeks (Q4W) dosing interval until the central subfield thickness (CST) meets a predefined reference CST threshold (of CST <325 μm for Spectralis spectral domain-central subfield thickness SD-OCT, or <315 μm for Cirrus SD-OCT or Topcon SD-OCT) (as measured at week 12 or later);
b) then the dosing interval is increased by 4 weeks to an initial every 8 weeks (Q8W) dosing interval;
c) from this point forward, the dosing interval is extended, reduced, or maintained based on assessments made at the dosing visits which are based on the relative change of the CST and best-corrected visual acuity (BCVA) compared with the respective reference CST and BCVA;
   wherein the
   i) interval is extended by 4 weeks,
      if the CST value is increased or decreased by ≤10% without an associated ≥10-letter BCVA decrease;
   ii) interval will be maintained:
      if the CST is decreased by >10%, or
      the CST value is increased or decreased by ≤10% with an associated ≥10-letter BCVA decrease, or
      the CST value is increased between >10% and ≤20% without an associated ≥5-letter BCVA decrease;
   iii) interval is reduced by 4 weeks
      if the CST value is increased between >10% and ≤20% with an associated ≥5 to <10-letter BCVA decrease; or
      the CST value is increased by >20% without an associated ≥10-letter BCVA decrease;
   iv) interval is reduced by 8 weeks if the CST value is increased by >10% with an associated ≥10-letter BCVA decrease;
wherein the respective reference central subfield thickness (CST) is the CST value when the initial CST threshold criteria are met and the reference CST is adjusted if CST decreases by >10% from the previous reference CST for two consecutive dosing visits and the values obtained are within 30 μm so that the CST value obtained at the latter visit will serve as the new reference CST; and
wherein the reference best-corrected visual acuity (BCVA) is the mean of the three best BCVA scores obtained at any prior dosing visit.

In one embodiment such dosing interval can by adjusted by 4-week increments to a maximum of every 16 weeks (Q16W) and a minimum of Q4W.

According to another aspect of the present invention, methods, uses, bispecific antibodies (for use), medicaments or pharmaceutical formulations are provided for the treatment of patients suffering from macular edema secondary to central retinal vein occlusion, secondary to hemiretinal vein occlusion or secondary to branch vein occlusion the method comprising administering to the patient an effective amount of a bispecific antibody which binds to human vascular endothelial growth factor (VEGF) and to human angiopoietin-2 (ANG-2) with personalized treatment interval (PTI) regimen wherein the treatment of patients suffering from macular edema secondary to central retinal vein occlusion, secondary to hemiretinal vein occlusion or secondary to branch vein occlusion includes a dosing schedule that extends the administration interval in stable absence of disease, or shortens the interval if there is disease activity. In such a way patients are optimally treated ensuring improvement and/or maintenance of their visual acuity and at the same time reducing unnecessary treatment burden.
One embodiment is such method, use, bispecific antibody (for use), medicament or pharmaceutical formulation for the treatment of patients suffering from macular edema secondary to central retinal vein occlusion, secondary to hemiretinal vein occlusion or secondary to branch vein occlusion the method comprising administering to the patient an effective amount of a bispecific antibody which binds to human vascular endothelial growth factor (VEGF) and to human angiopoietin-2 (ANG-2) with a personalized treatment interval, wherein
   a) patients are treated first with the bispecific VEGF/ANG2 antibody at an every 4 weeks (Q4W) dosing interval from Day 1 through Week 20
   b) from Week 24, patients receive the bispecific VEGF/ANG2 antibody at a frequency of Q4W until the central subfield thickness (CST) meets a predefined reference CST threshold;
   c) from this point forward, the dosing interval is extended, reduced, or maintained based on assessments made at the dosing visits which are based on the relative change of the CST and best-corrected visual acuity (BCVA) compared with the respective reference CST and BCVA; wherein the
      i) interval is extended by 4 weeks
      if the CST value is increased or decreased by ≤10% without an associated ≥10-letter BCVA decrease; or
      ii) interval is maintained if any of the following criteria are met:
      if the CST value is decreased by >10%; or
      if the CST value is decreased ≤10% with an associated ≥10-letter BCVA decrease; or
      if the CST value is increased between >10% and ≤20% without an associated ≥5-letter BCVA decrease;
      iii) interval is reduced by 4 weeks if any of the following criteria are met:
      if the CST value is increased between >10% and ≤20% with an associated ≥5-to <10-letter BCVA decrease, or
      if the CST value is increased by >20% without an associated ≥10-letter BCVA decrease, or
      if the CST value is increased by ≤10% with an associated BCVA decrease of ≥10-letters;
      iv) interval is reduced to Q4W
      if the CST value is increased by >10% with an associated ≥10-letter BCVA decrease,
         wherein the respective reference central subfield thickness (CST) is the CST value when the initial CST threshold criteria are met and the reference CST is adjusted if CST decreases by >10% from the previous reference CST for two consecutive dosing visits and the values obtained are within 30 μm so that the CST value obtained at the latter visit will serve as the new reference CST; and
         wherein the reference best-corrected visual acuity (BCVA) is the mean of the three best BCVA scores obtained at any prior dosing visit.
In one embodiment such dosing interval can by adjusted by 4-week increments to a maximum of every 16 weeks (Q16W) and a minimum of Q4W. In one embodiment of the invention the bispecific antibody which binds to human VEGF and to human ANG2 is a bispecific, bivalent anti-VEGF/ANG2 antibody comprising a first antigen-binding site that specifically binds to human VEGF and a second antigen-binding site that specifically binds to human ANG-2, wherein
   i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
   ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14, and wherein
   iii) the bispecific antibody comprises a constant heavy chain region of human IgG1 subclass comprising the mutations I253A, H310A, and H435A and the mutations L234A, L235A and P329G (numberings according to EU Index of Kabat).
In one embodiment of the invention the patients suffering from an ocular vascular disease have not been previously treated with anti-VEGF treatment (e.g. monotherapy) (are treatment naive).
In one embodiment of the invention the patients suffering from an ocular vascular disease have been previously treated with anti-VEGF treatment (e.g. monotherapy).
In one embodiment of the present invention, the disclosed bispecific antibody is administered according to determinations of a software tool.

### DESCRIPTION OF THE FIGURES

FIG. 1: FIG. 1 presents an overview of the study design for nAMD
a At Weeks 20 and 24, patients will undergo a disease activity assessment. Patients with anatomic or functional signs of disease activity at these time points will receive Q8W or Q12W dosing, respectively, rather than Q16W dosing.
b The primary endpoint is the change from baseline in BCVA (as assessed on the ETDRS chart at a starting distance of 4 meters) based on an average at Weeks 40, 44, and 48.
c From Week 60 (when all patients in Arm A are scheduled to receive faricimab) onward, patients in Arm A will be treated according to a PTI dosing regimen (between Q8W and Q16W).
BCVA=best-corrected visual acuity; ETDRS=Early Treatment Diabetic Retinopathy Study; IVT=intravitreal; PTI=personalized treatment interval; Q8W=every 8 weeks; Q12W=every 12 weeks; Q16W=every 16 weeks; W=Week.

FIG. 2: FIG. 2 presents an overview of the study design for DME
Arm A (administered Q8W): Patients randomized to Arm A will receive 6-mg IVT RO6867461 (faricimab) injections Q4W to Week 20, followed by 6-mg IVT RO6867461 (faricimab) injections Q8W to Week 96, followed by the final study visit at Week 100.
Arm B (personalized treatment interval PTI): Patients randomized to Arm B will receive 6-mg IVT RO6867461 (faricimab) injections Q4W to at least Week 12, followed by PTI dosing (see the PTI dosing criteria below) of 6-mg IVT RO6867461 (faricimab) injections to Week 96, followed by the final study visit at Week 100.
Arm C (comparator arm) (administered Q8W): Patients randomized to Arm C will receive 2-mg IVT aflibercept injections Q4W to Week 16, followed by 2-mg IVT aflibercept injections Q8W to Week 96, followed by the final study visit at Week 100.
Patients in all three treatment arms will complete scheduled study visits Q4W for the entire study duration (100 weeks). A sham procedure will be administered to patients in all three treatment arms at applicable visits to maintain masking among treatment arms
IVT=intravitreal; Q8W=every 8 weeks; PTI=personalized treatment interval (see section 3.1.2 for additional details); W=week.
a The definition of 1 year used for the primary efficacy endpoint—defined as the change from baseline in BCVA, as measured on the ETDRS chart at a starting distance of 4 meters at 1 year—is the average of the Week 48, 52, and 56 visits.

FIG. 3: Schematic Personalized treatment interval for DME—FIG. 3 outlines the algorithm for interval decision-making, which is based on the relative change of the CST and BCVA compared with reference CST and reference BCVA.
Significance of * and ** in FIG. 3:
Reference central subfield thickness (CST): the CST value when the initial CST threshold criteria are met. Reference CST is adjusted if CST decreases by >10% from the previous reference CST for two consecutive study drug dosing visits and the values obtained are within 30 μm. The CST value obtained at the latter visit will serve as the new reference CST, starting immediately at that visit.
Reference best-corrected visual acuity (BCVA): the mean of the three best BCVA scores obtained at any prior study drug dosing visit.

FIG. 4: Schematic comparison of durability (time to retreatment) in DME and nAMD and efficacy (DME) to other treatment options of DME and nAMD based on published results (Compared agents Lucentis® (ranibizumab), Eylea® (aflibercept), brolucizumab and VA2 (RO6867461/faricimab).

FIG. 5: BCVA gains from baseline of patients with neovascular age-related macular degeneration (nAMD) comparing the bispecific anti-VEGF/ANG2 antibody RO6867461 (faricimab) at 12- and 16-week intervals and ranibizumab (Lucentis®) at 4-week intervals.

FIG. 6: Time to necessary retreatment of diabetic macular edema (DME) based on disease activity assessed by both: BCVA decreased by ≥5 letters and CST increased by ≥50 μm (after dosing has discontinued (after 20 weeks or 6 monthly doses=Time post last intravitreal (IVT) administration). The bispecific anti-VEGF/ANG2 antibody RO6867461 (faricimab), was compared to ranibizumab (Lucentis®) and showed longer time to retreatment.

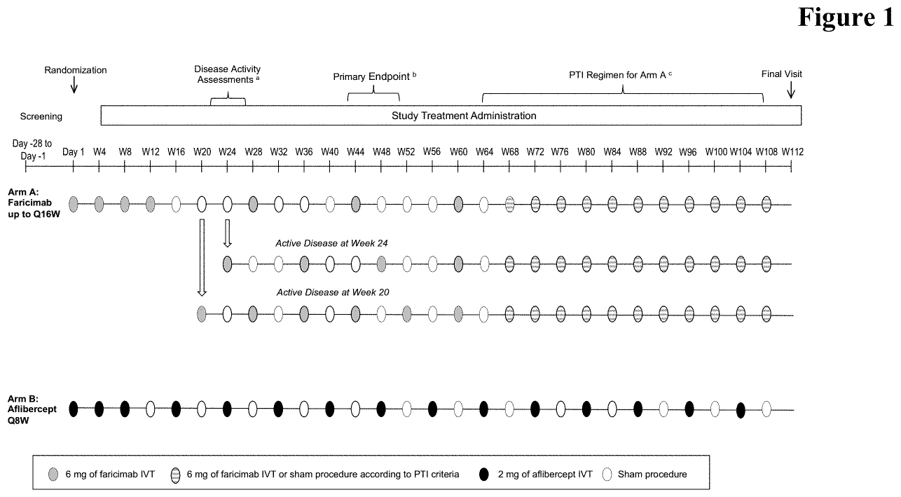

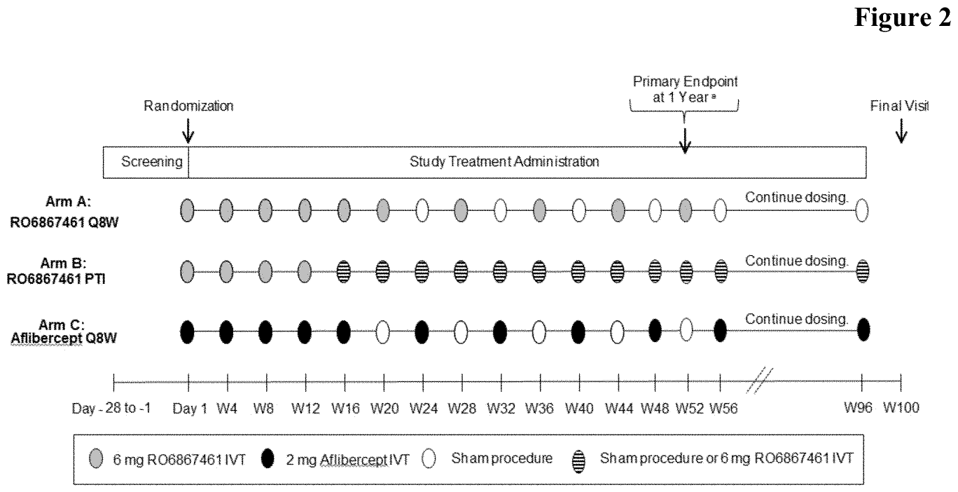

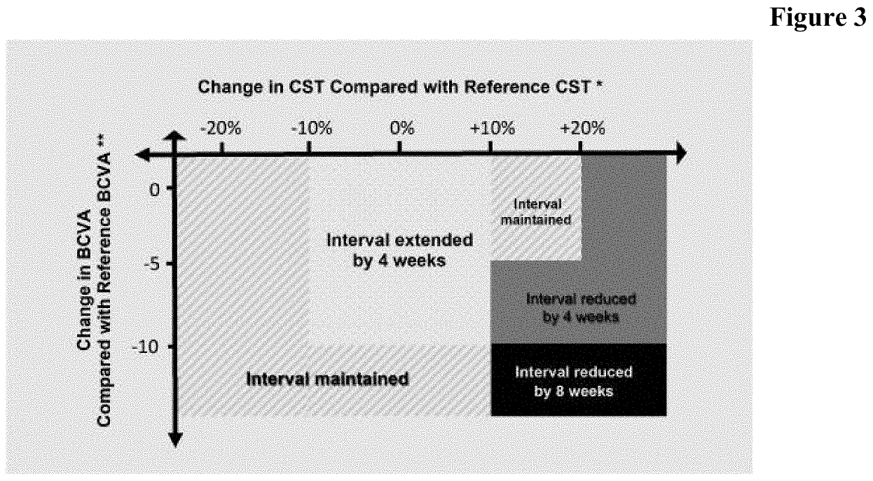

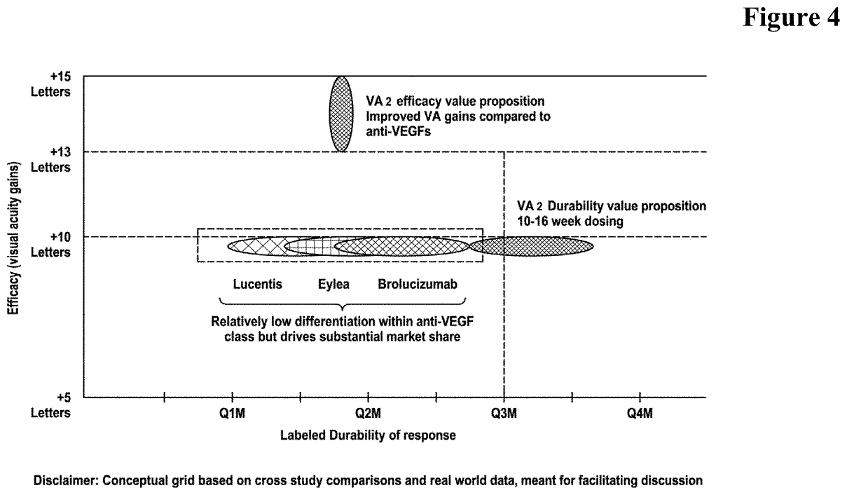

FIG. 7: FIG. 1 presents an overview of the study design for the treatment of macular edema secondary to retinal vein occlusion (RVO)
IVT=intravitreal; PTI=personalized treatment interval; Q4W=every 4 weeks; W=Week

FIG. 8: Schematic Personalized treatment interval for the treatment of macular edema secondary to retinal vein occlusion (RVO)—FIG. 8 outlines the algorithm for interval decision-making, which is based on the relative change of the CST and BCVA compared with reference CST and reference BCVA.
BCVA=best-corrected visual acuity; CST=central subfield thickness; Q4W=every 4 weeks.
a Initial reference CST=CST value when the initial CST threshold criteria are met, but no earlier than Week 20. Reference CST is adjusted if CST decreases by>10% from the previous reference CST for two consecutive faricimab dosing visits and the values obtained are within 30 μm. The CST value obtained at the latter visit will serve as the new reference CST, starting immediately at that visit.
b Reference BCVA=mean of the three best BCVA scores obtained at any prior dosing visit.

### DETAILED DESCRIPTION OF THE INVENTION

The method, use, bispecific antibody (for use), medicament or pharmaceutical formulation for use in the treatment of ocular vascular disease selected from nAMD and DME comprises sequentially administering initial doses (“treatment initiation”). In some embodiments the initial doses may vary , e.g. from 3 to 7 monthly administrations; in one embodiment the treatment initiation includes 3 to 4 monthly administrations, in one embodiment the treatment initiation includes 4 to 5 monthly administrations; in one embodiment the treatment initiation includes 4 to 6 monthly administrations; in one embodiment the treatment initiation includes at least 4 monthly administrations; in one embodiment the treatment initiation includes 5 to 7 monthly administrations, in one embodiment the treatment initiation includes 6 monthly administrations.

In one embodiment of the invention the bispecific antibody, medicament or pharmaceutical formulation is administered in a dose of about 5 to 7 mg (at each treatment). In one embodiment the bispecific antibody is administered in a dose of 6 mg+/−10% (at each treatment). In one embodiment the bispecific antibody is administered in a dose of about 6 mg (at each treatment) (in one embodiment in a dose of 6 mg (at each treatment)).

In one embodiment of the invention the bispecific antibody, medicament or pharmaceutical formulation is administered in a concentration of about 120 mg/ml (+/−12 mg/ml), of the bispecific antibody.

Macular degeneration is a medical condition predominantly found in elderly adults in which the center of the inner lining of the eye, known as the macula area of the retina, suffers thinning, atrophy, and in some cases, bleeding. This can result in loss of central vision, which entails inability to see fine details, to read, or to recognize faces. According to the American Academy of Ophthalmology, it is the leading cause of central vision loss (blindness) in the United States today for those over the age of fifty years. Although some macular dystrophies that affect younger individuals are sometimes referred to as macular degeneration, the term generally refers to age-related macular degeneration (AMD or ARMD).

“Age-related macular degeneration (AMD)”, as used herein, refers to a serious eye condition when the small central portion of the retina, known as the macula, deteriorates. AMD includes wet AMD and neovascular AMD. The wet form of AMD (wet AMD, wAMD or also called neovascular AMD, nAMD) is characterized by the growth of abnormal blood vessels from the choroid underneath the macula. This is called choroidal neovascularization. These blood vessels leak blood and fluid (below and) into the retina, causing (elevation of the retina and) distortion of vision that makes straight lines look wavy, as well as blind spots and loss of central vision. These abnormal blood vessels eventually scar, leading to permanent loss of central vision. The symptoms of AMD include dark, blurry areas in the center of vision; and diminished or changed color perception. AMD can be detected in a routine eye exam. One of the most common early signs of macular degeneration is the presence of drusen which are tiny yellow deposits under the retina and pigment clumping.

Advanced AMD, which is responsible for profound vision loss, has two forms: dry and wet. Central geographic atrophy, the dry form of advanced AMD, results from atrophy to the retinal pigment epithelial layer below the retina, which causes vision loss through loss of photoreceptors (rods and cones) in the central part of the eye.

While no treatment is available for this condition, vitamin supplements with high doses of antioxidants, lutein and zeaxanthin, have been demonstrated by the National Eye Institute and others to slow the progression of dry macular degeneration and in some patients, improve visual acuity.

“Diabetic Macular Edema” (DME), as used herein, refers to a serious eye condition that affects people with diabetes (type 1 or 2). Macular edema occurs when blood vessels in the retina leak into the macula and fluid and protein deposits collect on or under the macula of the eye and causes it to thicken and swell (edema). The swelling may distort a person's central vision, as the macula is near the center of the retina at the back of the eyeball. The primary symptoms of DME include, but are not limited to, blurry vision, floaters, loss of contrast, double vision, and eventual loss of vision. The pathology of DME is characterized by breakdown of inner the blood-retinal barrier, normally preventing fluid movement in the retina, thus allowing fluid to accumulate in the retinal tissue, and presence of retinal thickening. DME is presently diagnosed during an eye examination consisting of a visual acuity test, which determines the smallest letters a person can read on a standardized chart, a dilated eye exam to check for signs of the disease, imaging tests such as optical coherence tomography (OCT) or fluorescein angiography (FA) and tonometry, an instrument that measures pressure inside the eye. The following studies are also performed to determine treatment: optical coherence tomography (OCT), fluorescein angiography, and color stereo fundus photography. DME can be broadly characterized into two main categories—Focal and Diffuse. Focal DME is characterized by specific areas of separate and distinct leakage in the macula with sufficient macular blood flow. Diffuse DME results from leakage of the entire capillary bed surrounding the macula, resulting from a breakdown of the inner blood-retina barrier of the eye. In addition to Focal and Diffuse, DME is also categorized based on clinical exam findings into clinically significant macular edema (CSME), non-CSME and CSME with central involvement (CSME-CI), which involves the fovea. The present invention includes methods to treat the above-mentioned categories of DME.

Retinal vein occlusion (RVO) is one of the most common retinal vascular disorders and is associated with varying degrees of visual loss (Hayreh and Zimmerman 1994). RVO has been reported as the second leading cause of blindness for patients with retinal vascular disease, following diabetic retinopathy (DR) (Cugati S, Wang J J, Rochtchina E, et al. Arch Ophthalmol 2006; 124 :726-732; Klein R, Knudtson M D, Lee K E, et al. Ophthalmology 2008; 115 :1859-1868; Rogers S, McIntosh R L, Cheung N, et al. Ophthalmology 2010 Feb; 117:313-9.el; Yasuda M, Kiyohara Y, Arakawa S, et al. Invest Ophthalmol Vis Sci 2010; 51:3205-3209).

The main types of RVO include branch retinal vein occlusion (BRVO), hemiretinal vein occlusion (HRVO), and central retinal vein occlusion (CRVO). The most common presenting complaint of RVO is an abrupt, painless decrease of central vision due to macular edema.

The main types of macular edema secondary to RVO include macular edema secondary to branch retinal vein occlusion (BRVO), macular edema secondary to hemiretinal vein occlusion (HRVO), and macular edema secondary to central retinal vein occlusion (CRVO).

Less frequently, patients may present with a history of transient vision loss, lasting a few seconds to minutes, with complete recovery of vision. These symptoms may recur over several days to weeks, followed by a permanent decrease in vision.

Metamorphopsia and visual field defects have also been described (Achiron A, Lagstein O, Glick M, et al. Acta Ophthalmologica 2015; 93:e649-53; Manabe K, Osaka R, Nakano Y, et al. PLoS One 2017; 12 :e0186737).

The pathogenesis of macular edema in these patients starts with an increase in intraluminal pressure due to vascular obstruction, which causes areas of reduced perfusion and ischemia. Ischemia leads to up-regulation and secretion of vascular endothelial growth factor (VEGF) (Boyd S R, Zachary I, Chakravarthy U, et al. Arch Ophthalmol 2002; 12:1644-1650; Noma H, Minamoto A, Funatsu H, et al. Graefes Arch Clin Exp Ophthalmol 2006; 244:309-315) and angiopoietin-2 (Ang-2), both well-known proangiogenic and vessel hyperpermeability cytokines with Ang-2 contributing additional pro-inflammatory and vessel destabilization properties (Maisonpierre P C, Suri C, Jones P F, et al. Science 1997; 277:55-60; Hackett S F, Ozaki H, Strauss R W, et al. J Cell Physiol 2000; 184 :275-284; Fiedler U, Reiss Y, Scharpfenecker M, et al. Nat Med 2006; 12:235-239. Epub: 5 Feb. 2006). Patients with RVO were found to have the highest vitreous levels of both Ang-2 and VEGF among all retinal vascular diseases (Aiello L P, Avery R L, Arrigg P G, et al. N Engl J Med 1994; 331:1480-1487; Regula J T, Lundh von Leithner P, Foxton R, et al. EMBO Mol Med 2016; 8:1265-1288). Increased levels of Ang-2 and VEGF in retinal tissue results in pathological changes in the retina and, in many patients, also macular edema accompanied with decrease in vision. A hallmark of RVO is the characteristic pattern of retinal hemorrhages, tortuous and dilated retinal veins across the affected area of retina (one quadrant in BRVO, two quadrants in HRVO and the entire retina in CRVO). In more severe cases, patients can develop retinal ischemia with subsequent retinal neovascularization, hemorrhages, neovascularization in the anterior segment leading to rubeosis or neovascular glaucoma, and some patients may develop optic disc edema.

Although macular edema due to RVO and diabetic macular edema (DME) have different origins, they share a common pathophysiology. Both are characterized by a thickening of the macula due to fluid accumulation consequent to breakdown of the blood-retinal barrier and a pathological increase of retinal vessel permeability, which can lead to irreversible vision loss in both diseases.

Anti-VEGF pharmacotherapy is the current mainstay of treatment in macular edema due to RVO and has demonstrated efficacy across several pivotal, randomized clinical studies, although macular laser and intravitreal (IVT) steroids—especially steroid implants—are also used in some cases. Despite anti-VEGF being the most effective therapy for macular edema due to RVO, data from anti-VEGF clinical trials showed that many patients do not achieve optimal best-corrected visual acuity (BCVA) and anatomical outcomes, and many require frequent long-term injections to maintain the gains achieved during initial intensive treatment. Moreover, real-world data analyses suggested that many patients with RVO do not achieve the gains reached in clinical trials due to suboptimal injection frequency (Vaz-Pereira, S, Marques I P, Matias J, et al. Eur J Ophthalmol 2017; 27:756-761; Wecker T, Ehlken C, Buhler A, et al. Br J Ophthalmol 2017; 101:353-359; Jumper J M, Dugel P U, Chen S, et al. Clin Ophthalmol 2018; 12:621-629). The data suggest that many patients with macular edema due to BRVO and the majority of patients with macular edema due to CRVO require close monitoring and treatment for a longer period of time and that more durable and efficacious treatment options are needed (Bhisitkul R B, Campochiaro P A, Shapiro H, et al. Ophthalmology 2013; 120:1057-1063; Scott I U, Neal N L, VanVeldhuisen, et al. JAMA Ophthalmol 2019; E1-E10).

Nonclinical studies have shown that Ang-2 and VEGF act in concert to regulate the vasculature and to increase retinal endothelial cell permeability in vitro. Simultaneous inhibition of Ang-2 and VEGF with the bispecific monoclonal antibody faricimab led to a greater reduction in the leakiness and severity of choroidal neovascularization (CNV) lesions in a laser-induced CNV model in non-human primates compared with the molar equivalent of anti-VEGF (ranibizumab) or anti-Ang-2 alone. Earlier experiments using a mouse model of spontaneous CNV showed that dual inhibition of Ang-2 and VEGF consistently outperformed monotherapeutic inhibition of either target alone in terms of reduction in vascular growth, leakage, edema, leukocyte infiltration, and photoreceptor loss (Regula J T, Lundh von Leithner P, Foxton R, et al. EMBO Mol Med 2016; 8:1265-1288). In addition, aqueous and vitreous concentrations of both Ang-2 and VEGF were shown to be upregulated in patients with neovascular age-related macular degeneration (nAMD), DR, and RVO (Tong J P, Chan W M, Liu D T, et al. Am J Ophthalmol 2006; 141:456-462; Penn J S, Madan A, Caldwell R B, et al. Prog Retin Eye Res 2008; 27:331-371.; Kinnunen K, Puustjärvi T, Terasvirta M, et al. Br J Ophthalmol 2009; 93:1109-1115; Tuuminen R, Loukovaara S. Eye (Lond) 2014; 28 :1095-1099; Regula J T, Lundh von Leithner P, Foxton R, et al. EMBO Mol Med 2016; 8:1265-1288; Ng D S, Yip Y W, Bakthavatsalam M, et al. Sci Rep 2017; 7:45081). Therefore, simultaneous neutralization of both targets, Ang-2 and VEGF, may further normalize the pathological ocular vasculature compared with anti-VEGF therapy alone. Data from the completed Phase II studies in DME and nAMD (see below) also support the hypothesis that targeting Ang-2 has the potential to extend the durability of effect beyond anti-VEGF therapy alone in diseases affecting the retinal vasculature.

Faricimab has been studied for the treatment of nAMD and DME in two Phase I studies (BP28936 in nAMD and JP39844 in nAMD and DME) and in three Phase II studies (BP29647 [AVENUE] and CR39521 [STAIRWAY] for nAMD and BP30099 [BOULEVARD] for DME). Four global Phase III studies are ongoing: GR40349 (YOSEMITE) and GR40398 (RHINE) in DME and GR40306 (TENAYA) and GR40844 (LUCERNE) in nAMD.

Based on the mechanism of action of faricimab, data from nonclinical and clinical trials, and the pathophysiology of macular edema due to RVO, it is hypothesized that faricimab may lead to stabilization of the pathological ocular vasculature and to improved visual and anatomical outcomes in RVO compared with anti-VEGF monotherapies.

Macular edema secondary to/due to RVO are among the highest in retinal vascular diseases (Aiello L P, Avery R L, Arrigg P G, et al. N Engl J Med1994; 331:1480-1487; Regula J T, Lundh von Leithner P, Foxton R, et al. EMBO Mol Med 2016; 8:1265-1288). The effect of Ang-2 and VEGF inhibition in the nonclinical models of angiogenesis and inflammation (Regula J T, Lundh von Leithner P, Foxton R, et al. EMBO Mol Med 2016; 8:1265-1288) and the data from Phase I and Phase II faricimab studies in patients with nAMD and DME provide the evidence of efficacy on pathological pathways that are common to all three retinal vascular diseases: nAMD, DME/DR, and macular edema due to RVO (Phase I study BP28936 in nAMD; Phase II studies AVENUE in nAMD, STAIRWAY in nAMD, and BOULEVARD in DME).

Data from the Phase II BOULEVARD study are reported here due to parallels in pathophysiology between DME and macular edema due to RVO. While the trigger for macular edema in diabetic and RVO patients is different, the downstream pathophysiology of hypoxia-driven macular edema with subsequent vision loss is similar and driven by the same proangiogenic, pro-inflammatory, vessel destabilization and vessel permeability factors, including Ang-2, VEGF, and interleukin-6 (IL-6). The BOULEVARD study provided preliminary evidence of a positive benefit-risk profile for the use of 6-mg IVT injections of faricimab for patients with DME and supported further evaluation of faricimab in the Phase III DME studies. The study met its primary efficacy endpoint, demonstrating statistically significant improvement in the mean change from baseline in BCVA at Week 24 in patients naive to anti-VEGF treatment who were treated with 6 mg faricimab compared with 0.3 mg ranibizumab. Best Corrected Visual Acuity (BCVA) is determined using methodology adapted from the 4-meter Early Treatment Diabetic Retinopathy Study [ETDRS] protocol (using Early Treatment Diabetic Retinopathy Study (ETDRS) like charts) and resulting in the respective letter score. In one embodiment BCVA determination in such method, use, bispecific antibody (for use), medicament or pharmaceutical formulation is based on the Early Treatment of Diabetic Retinopathy Study (ETDRS) Protocol adapted visual acuity charts and is assessed at a starting distance of 4 meters.

Disease activity is determined e.g. via reduction of the BCVA/ETDRs letter score and/or e.g. via the macular thickening by spectral domain optical coherence tomography (SD-OCT) involving the center of the macula as central subfield thickness (CST) (also known as center subfoveal thickness). In one preferred embodiment Central Subfield Thickness (CST) is determined using spectral domain optical coherence tomography (SD-OCT): In one preferred embodiment CST is measured by spectral domain optical coherence tomography (SD-OCT) with a Spectralis™ device; in one preferred embodiment CST is measured by spectral domain optical coherence tomography (SD-OCT) with a Cirrus' device; in one embodiment CST is measured by spectral domain optical coherence tomography (SD-OCT) with a Topcon™ device; in one embodiment CST is measured by spectral domain optical coherence tomography (SD-OCT) with a Optovue™ device). As used herein, the term “a patient suffering from” refers to a human that exhibits one or more symptoms or indications of, and/or who has been diagnosed with an ocular vascular disease as described herein. The term “a patient suffering from” may also include, e.g., subjects who, prior to treatment, exhibit (or have exhibited) one or more indications of a vascular eye disease such as, e.g., retinal angiogenesis, neovascularization, vascular leak, retinal thickening of the center of the fovea, hard, yellow exudates of the center of the fovea with adjacent retinal thickening, and at least 1 disc area of retinal thickening, any part of which is within 1 disc diameter of the center of the fovea, blurry vision, floaters, loss of contrast, double vision, and eventual loss of vision.

As used herein, the term “a patient suffering from” an ocular vascular disease such as nAMD or DME may include a subset of population which is more susceptible to nAMD or DME or may show an elevated level of a nAMD-associated or DME associated biomarker. For example, “a patient suffering from DME” may include a subject suffering from diabetes for more than 10 years, have frequent high blood sugar levels or high fasting blood glucose levels. In certain embodiments, the term “a patient suffering from DME” includes a subject who, prior to or at the time of administration of the bispecific anti-VEGF/ANG2 antibody, has or is diagnosed with diabetes. In certain embodiments, the term “a patient suffering from nAMD” includes a subject who, prior to or at the time of administration of the anti-VEGF/ANG2 antibody, is more than 50 years old. In some embodiments, the term “a patient suffering from” includes subjects who are smokers, or subjects with high blood pressure or high cholesterol.

As used herein, the term “a patient suffering from” an ocular vascular disease such as macular edema secondary to branch retinal vein occlusion (BRVO), macular edema secondary to hemiretinal vein occlusion (HRVO), or macular edema secondary to central retinal vein occlusion (CRVO)may include a subset of population which is more susceptible to macular edema secondary to branch retinal vein occlusion (BRVO), macular edema secondary to hemiretinal vein occlusion (HRVO), or macular edema secondary to central retinal vein occlusion (CRVO) or may show an elevated level of a RVO-associated biomarker. For example, “a patient suffering from RVO or macular edema secondary to RVO” may include a subject with increased levels of VEGF, ANG2 or IL-6. In some embodiments, the term “a patient suffering from” includes subjects who are smokers, or subjects with high blood pressure or high cholesterol. The present invention includes methods or bispecific antibodies (for use), medicaments or pharmaceutical formulations for treating, preventing or reducing the severity of an ocular vascular disease comprising administering a therapeutically effective amount of a bispecific anti-VEGF/ANG2 antibody (or a medicament or pharmaceutical formulation comprising the bispecific anti-VEGF/ANG2 antibody) to a subject in need thereof, wherein the bispecific antibody, medicament or pharmaceutical formulation comprising such bispecific anti-VEGF/ANG2 antibody is administered (intravitreally) to the subject in multiple doses, e.g., as part of a specific therapeutic dosing regimen.
One embodiment of the invention is the method of treatment, use, bispecific antibody (for use), medicament or pharmaceutical formulation as described herein wherein patients suffering from an ocular vascular disease have not been previously treated with anti-VEGF treatment (e.g. monotherapy) (are treatment naïve).
One embodiment of the invention is the method of treatment, use, bispecific antibody (for use), medicament or pharmaceutical formulation as described herein wherein patients suffering from an ocular vascular disease have been previously treated with anti-VEGF treatment (e.g. monotherapy, e.g., with ranibizumab, aflibercept or brolocizumab).
One embodiment of the invention is a method, use, bispecific antibody (for use), medicament or pharmaceutical formulation for use in the treatment of patients suffering from neovascular AMD (nAMD) the method comprising administering to the patient an effective amount of a bispecific antibody which binds to human vascular endothelial growth factor (VEGF) and to human angiopoietin-2 (ANG-2) with a personalized treatment interval, wherein
a) patients are treated first 4 times with the bispecific VEGF/ANG2 antibody at an every 4 weeks (Q4W) dosing interval;
b) at Weeks 20 and 24 the disease activity is assessed wherein the disease activity is determined if one of the following criteria are met:
   i) increase of >50 μm in central subfield thickness (CST) compared with the average CST value over the previous two scheduled visits which are Weeks 12 and 16 for the Week 20 assessment, and Weeks 16 and 20 for the Week 24 assessment, or
   ii) increase 75 μm in CST compared with the lowest CST value recorded at either of the previous two scheduled visits;
   iii) decrease 5 letters in best-corrected visual acuity (BCVA) compared with average BCVA value over the previous two scheduled visits, owing to nAMD disease activity,
   iv) decrease 10 letters in BCVA compared with the highest BCVA value recorded at either of the previous two scheduled visits, owing to nAMD disease activity, or
   v) presence of new macular hemorrhage, owing to nAMD activity
c) then patients
   i) patients who meet the disease activity criteria at Week20 will be treated at a Q8W dosing interval from week 20 onward (with the first Q8W dosing at Week20);
   ii) patients who meet the disease activity criteria at Week24 will be treated at a Q12W dosing interval from week 24 onward (with the first Q12W dosing at Week24); and
   iii) patients who do not meet disease activity criteria at Week20 and Week24 will be treated at a Q16W dosing interval from week 28 onward (with the first Q16W dosing at Week28).
In one embodiment the personalized treatment interval will be extended, reduced, or maintained after week 60 wherein the
   a) interval is extended by 4 weeks (to a maximum of Q16W) if all of the following criteria are met:
      i) stable CST compared with the average of the last 2 study drug dosing visits where stability is defined as a change of CST of less than 30 μm and no increase ≥50 μm in CST compared with the lowest on-study drug dosing visit measurement,
      ii) no decrease ≥5 letters in BCVA compared with the average from the last two study drug dosing visits, and no decrease ≥10 letters in BCVA compared with the highest on-study drug dosing visit measurement,
      iii) no new macular hemorrhage;
   b) interval
      is reduced (to a minimum Q8W) by 4 weeks if one of the following criteria is met,
      or
      is reduced to an 8-week interval if two or more of the following criteria are met or one criterion includes new macular hemorrhage:
      i) increase of ≥50 μm in CST compared with the average from the last two dosing visits or of ≥75 μm compared with the lowest dosing visit measurement,
      ii) decrease of ≥5 letters in BCVA compared with average of last two dosing visits or decrease ≥10 letters in BCVA compared with the highest dosing visit measurement,
      iii) new macular hemorrhage.
In one embodiment the disease activity assessment before the personalized treatment interval will be at Weeks 16 and Week 20, or at Weeks 24 and Week 28.
In one embodiment the personalized treatment interval with further extension, reduction, or maintenance will start at a different time point e.g. between after week 50 and 70, e.g. after week 52 or after week 65 depending on the disease activity. Another embodiment of the invention is a method, use, bispecific antibody (for use), medicament or pharmaceutical formulation for use in the treatment of patients suffering from diabetic macular edema (DME) the method comprising administering to the patient an effective amount of a bispecific antibody which binds to human vascular endothelial growth factor (VEGF) and to human angiopoietin-2 (ANG-2) with a personalized treatment interval, wherein
a) patients are treated first with the bispecific VEGF/ANG2 antibody at an every 4 weeks (Q4W) dosing interval until the central subfield thickness (CST) meets a predefined reference CST threshold (of CST <325 μm for Spectralis spectral domain-central subfield thickness SD-OCT, or <315 μm for Cirrus SD-OCT or Topcon SD-OCT) (as measured at week 12 or later);
b) then the dosing interval is increased by 4 weeks to an initial Q8W dosing interval;
c) from this point forward, the dosing interval is extended, reduced, or maintained based on assessments made at the dosing visits. which are based on the relative change of the CST and best-corrected visual acuity (BCVA) compared with the respective reference CST and BCVA;
   wherein the
   i) interval is extended by 4 weeks,
      if the CST value is increased or decreased by ≤10% without an associated ≥10-letter BCVA decrease;
   ii) interval will be maintained:
      if the CST is decreased by >10%, or
      the CST value is increased or decreased by ≤10% with an associated ≥10-letter BCVA decrease, or
      the CST value is increased between >10% and ≤20% without an associated ≥5-letter BCVA decrease;
   iii) interval is reduced by 4 weeks
      if the CST value is increased between >10% and ≤20% with an associated ≥5 to <10-letter BCVA decrease; or
      the CST value is increased by >20% without an associated ≥10-letter BCVA decrease;
   iv) interval is reduced by 8 weeks if the CST value is increased by >10% with an associated >10-letter BCVA decrease;
wherein the respective reference central subfield thickness (CST) is the CST value when the initial CST threshold criteria are met and the reference CST is adjusted if CST decreases by >10% from the previous reference CST for two consecutive dosing visits and the values obtained are within 30 μm so that the CST value obtained at the latter visit will serve as the new reference CST; and
wherein the reference best-corrected visual acuity (BCVA) is the mean of the three best BCVA scores obtained at any prior dosing visit.

In one embodiment such dosing interval can by adjusted by 4-week increments to a maximum of every 16 weeks (Q16W) and a minimum of Q4W.
Another embodiment of the invention is a method, use, bispecific antibody (for use), medicament or pharmaceutical formulation for use in the treatment of patients suffering from an ocular vascular disease selected from macular edema secondary to central retinal vein occlusion, secondary to hemiretinal vein occlusion or secondary to branch vein occlusion, or of patients suffering from an ocular vascular disease selected from macular edema secondary to central retinal vein occlusion, secondary to hemiretinal vein occlusion or secondary to branch vein occlusion, wherein the treatment includes a personalized treatment interval (PTI), wherein
   a) patients are treated first with the bispecific VEGF/ANG2 antibody at an every 4 weeks (Q4W) dosing interval from Day 1 through Week 20
   b) from Week 24, patients receive the bispecific VEGF/ANG2 antibody at a frequency of Q4W until the central subfield thickness (CST) meets a predefined reference CST threshold (of CST <325 μm for Spectralis spectral domain-central subfield thickness SD-OCT, or <315 μm for Cirrus SD-OCT or Topcon SD-OCT) (as measured at week 24 or later);
   c) from this point forward, the dosing interval is extended, reduced, or maintained based on assessments made at the dosing visits which are based on the relative change of the CST and best-corrected visual acuity (BCVA) compared with the respective reference CST and BCVA; wherein the
      i) interval is extended by 4 weeks
      if the CST value is increased or decreased by ≤10% without an associated ≥10-letter BCVA decrease; or
      ii) interval is maintained if any of the following criteria are met:
      if the CST value is decreased by >10%; or
      if the CST value is decreased ≤10% with an associated ≥10-letter BCVA decrease; or
      if the CST value is increased between >10% and ≤20% without an associated ≥5-letter BCVA decrease;
      iii) interval is reduced by 4 weeks if any of the following criteria are met:
      if the CST value is increased between >10% and ≤20% with an associated ≥5-to <10-letter BCVA decrease, or
      if the CST value is increased by >20% without an associated ≥10-letter BCVA decrease, or
      if the CST value is increased by ≤10% with an associated BCVA decrease of ≥10-letters;
      iv) interval is reduced to Q4W
      if the CST value is increased by >10% with an associated ≥10-letter BCVA decrease,
   wherein the respective reference central subfield thickness (CST) is the CST value when the initial CST threshold criteria are met and the reference CST is adjusted if CST decreases by >10% from the previous reference CST for two consecutive dosing visits and the values obtained are within 30 μm so that the CST value obtained at the latter visit will serve as the new reference CST; and
   wherein the reference best-corrected visual acuity (BCVA) is the mean of the three best BCVA scores obtained at any prior dosing visit.

In one embodiment such dosing interval can by adjusted by 4-week increments to a maximum of every 16 weeks (Q16W) and a minimum of Q4W. As used herein, “antibody” refers to a binding protein that comprises antigen-binding sites. The terms “binding site” or “antigen-binding site” as used herein denotes the region(s) of an antibody molecule to which a ligand actually binds. The term “antigen-binding site” comprises an antibody heavy chain variable domains (VH) and an antibody light chain variable domains (VL) (pair of VH/VL).).

Antibody specificity refers to selective recognition of the antibody for a particular epitope of an antigen. Natural antibodies, for example, are monospecific.

“Bispecific antibodies” according to the invention are antibodies which have two different antigen-binding specificities. Antibodies of the present invention are specific for two different antigens, VEGF as first antigen and ANG-2 as second antigen.

The term “monospecific” antibody as used herein denotes an antibody that has one or more binding sites each of which bind to the same epitope of the same antigen.

The term “valent” as used within the current application denotes the presence of a specified number of binding sites in an antibody molecule. As such, the terms “bivalent”, “tetravalent”, and “hexavalent” denote the presence of two binding site, four binding sites, and six binding sites, respectively, in an antibody molecule. The bispecific antibodies according to the invention are preferably “bivalent”.

The terms “bispecific antibody which binds to human vascular endothelial growth factor (VEGF) and to human angiopoietin-2 (ANG-2)”, “bispecific anti-VEGF/ANG2 antibody” and bispecific <VEGF/ANG2> antibody” as used herein are interchangeable and refer to an antibody which has at least two different antigen-binding sites, a first one which binds to VEGF and a second one which binds to ANG2.

Bispecific anti-VEGF/ANG2 antibodies are e.g. described in WO2010040508, WO2011/117329, WO2012/131078, WO2015/083978, WO2017/197199, and WO2014/009465. WO2014/009465 describes bispecific anti-VEGF/ANG2 antibodies especially designed for treatment of ocular vascular diseases. The bispecific anti-VEGF/ANG2 antibodies of WO2014/009465 (which is incorporated herein in its entirety) are especially useful in the treatment and treatment schedules of ocular vascular diseases as described herein.

In one embodiment the bispecific antibody which binds to human vascular endothelial growth factor (VEGF) and to human angiopoietin-2 (ANG-2) is a bispecific anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14, and wherein
iii) the bispecific antibody comprises a constant heavy chain region of human IgG1 subclass comprising the mutations I253A, H310A, and H435A and the mutations L234A, L235A and P329G (numberings according to EU Index of Kabat).

In one embodiment such bispecific anti-VEGF/ANG2 antibody is bivalent.
In one embodiment such bispecific anti-VEGF/ANG2 antibody is characterized in that
   i) said first antigen-binding site specifically binding to VEGF comprises as heavy chain variable domain VH an amino acid sequence of SEQ ID NO: 7, and as light chain variable domain VL an amino acid sequence of SEQ ID NO: 8, and
   ii) said second antigen-binding site specifically binding to ANG-2 comprises as heavy chain variable domain VH an amino acid sequence of SEQ ID NO: 15, and as light chain variable domain VL an amino acid sequence of SEQ ID NO: 16.

In one aspect of the invention such bispecific, bivalent antibody according to the invention is characterized in comprising
a) the heavy chain and the light chain of a first full length antibody that specifically binds to VEGF;
b) the modified heavy chain and modified light chain of a second full length antibody that specifically binds to ANG-2, wherein the constant domains CL and CH1 are replaced by each other.

This bispecific, bivalent antibody format for the bispecific antibody specifically binding to human vascular endothelial growth factor (VEGF) and human angiopoietin-2 (ANG-2) is described in WO 2009/080253 (including Knobs-into-Holes modified CH3 domains). The antibodies based on this bispecific, bivalent antibody format are named CrossMAbs.

In one embodiment such bispecific, bivalent anti-VEGF/ANG2 antibody is characterized in comprising
a) as heavy chain of the first full length antibody the amino acid sequence of SEQ ID NO: 17, and as light chain of the first full length antibody the amino acid sequence of SEQ ID NO: 18, and
b) as modified heavy chain of the second full length antibody the amino acid sequence of SEQ ID NO: 19, and as modified light chain of the second full length antibody the amino acid sequence of SEQ ID NO: 20.
In one embodiment such bispecific, bivalent anti-VEGF/ANG2 antibody is characterized in comprising the amino acid sequences of SEQ ID NO: 17, of SEQ ID NO: 18, of SEQ ID NO: 19, and of SEQ ID NO: 20. In one preferred embodiment the bispecific, bivalent anti-VEGF/ANG2 antibody is faricimab.

Accordingly, one embodiment of the invention is a bispecific, bivalent antibody comprising a first antigen-binding site that specifically binds to human VEGF and a second antigen-binding site that specifically binds to human ANG-2, characterized in comprising the amino acid sequences of SEQ ID NO: 17, of SEQ ID NO: 18, of SEQ ID NO: 19, and of SEQ ID NO: 20. In one preferred embodiment the bispecific, bivalent anti-VEGF/ANG2 antibody is faricimab.

In on embodiment the CH3 domains of the bispecific, bivalent antibody according to the invention is altered by the “knob-into-holes” technology which is described in detail with several examples in e.g. WO 96/027011, Ridgway J. B., et al., Protein Eng 9 (1996) 617-621; and Merchant, A. M., et al., Nat Biotechnol 16 (1998) 677-681. In this method the interaction surfaces of the two CH3 domains are altered to increase the heterodimerisation of both heavy chains containing these two CH3 domains. Each of the two CH3 domains (of the two heavy chains) can be the “knob”, while the other is the “hole”. The introduction of a disulfide bridge stabilizes the heterodimers (Merchant, A. M, et al., Nature Biotech 16 (1998) 677-681; Atwell, S., et al. J. Mol. Biol. 270 (1997) 26-35) and increases the yield.

In a preferred aspect of the invention the bispecific anti-VEGF/ANG2 antibodies according to the invention are characterized in that

the CH3 domain of one heavy chain and the CH3 domain of the other heavy chain each meet at an interface which comprises an original interface between the antibody CH3 domains;

wherein said interface is altered to promote the formation of the bispecific antibody, wherein the alteration is characterized in that:

a) the CH3 domain of one heavy chain is altered,

so that within the original interface the CH3 domain of one heavy chain that meets the original interface of the CH3 domain of the other heavy chain within the bispecific antibody,

an amino acid residue is replaced with an amino acid residue having a larger side chain volume, thereby generating a protuberance within the interface of the CH3 domain of one heavy chain which is positionable in a cavity within the interface of the CH3 domain of the other heavy chain

and

b) the CH3 domain of the other heavy chain is altered,

so that within the original interface of the second CH3 domain that meets the original interface of the first CH3 domain within the bispecific antibody

an amino acid residue is replaced with an amino acid residue having a smaller side chain volume, thereby generating a cavity within the interface of the second CH3 domain within which a protuberance within the interface of the first CH3 domain is positionable.
Thus the bispecific anti-VEGF/ANG2 antibodies for use described herein are preferably characterized in that
   the CH3 domain of the heavy chain of the full length antibody of a) and the CH3 domain of the heavy chain of the full length antibody of b) each meet at an interface which comprises an alteration in the original interface between the antibody CH3 domains;
   wherein i) in the CH3 domain of one heavy chain
   an amino acid residue is replaced with an amino acid residue having a larger side chain volume, thereby generating a protuberance within the interface of the CH3 domain of one heavy chain which is positionable in a cavity within the interface of the CH3 domain of the other heavy chain and wherein
   ii) in the CH3 domain of the other heavy chain
   an amino acid residue is replaced with an amino acid residue having a smaller side chain volume, thereby generating a cavity within the interface of the second CH3 domain within which a protuberance within the interface of the first CH3 domain is positionable.

Preferably said amino acid residue having a larger side chain volume is selected from the group consisting of arginine (R), phenylalanine (F), tyrosine (Y), tryptophan (W). Preferably said amino acid residue having a smaller side chain volume is selected from the group consisting of alanine (A), serine (S), threonine (T), valine (V).

In one aspect of the invention both CH3 domains are further altered by the introduction of cysteine (C) as amino acid in the corresponding positions of each CH3 domain such that a disulfide bridge between both CH3 domains can be formed. In one embodiment, the bispecific antibody comprises a T366W mutation in the CH3 domain of the “knobs chain” and T366S, L368A, Y407V mutations in the CH3 domain of the “hole chain”. An additional interchain disulfide bridge between the CH3 domains can also be used (Merchant, A. M, et al., Nature Biotech 16 (1998) 677-681) e.g. by introducing a S354C mutation into one CH3 domain and a Y349C mutation into the other CH3 domain.

In a another preferred embodiment the bispecific antibody comprises S354C and T366W mutations in one of the two CH3 domains and Y349C, T366S, L368A, Y407V mutations in the other of the two CH3 domains In a another preferred embodiment the bispecific antibody comprises Y349C, T366W mutations in one of the two CH3 domains and S354C, T366S, L368A, Y407V mutations in the other of the two CH3 domains (the additional Y349C or S354C mutation in one CH3 domain and the additional S354C or Y349C mutation in the other CH3 domain forming a interchain disulfide bridge) (numbering always according to EU index of Kabat (Kabat, E. A., et al., Sequences of Proteins of Immunological Interest, 5th ed., Public Health Service, National Institutes of Health, Bethesda, Md. (1991)).

Other techniques for CH3-modifications to enforce the heterodimerization are contemplated as alternatives of the invention and described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954 and WO 2013/096291.

In one embodiment the heterodimerization approach described in EP 1 870 459A1 is used alternatively. This approach is based on the introduction of substitutions/mutations of charged amino acids with the opposite charge at specific amino acid positions of the in the CH3/CH3 domain interface between both heavy chains. One preferred embodiment for said multispecific antibodies are amino acid R409D and K370E mutations in the CH3 domain of one heavy chain and amino acid D399K and E357K mutations in the CH3 domain of the other heavy chain of the multispecific antibody (numberings according to Kabat EU index).

In another embodiment said multispecific antibody comprises an amino acid T366W mutation in the CH3 domain of the “knobs chain” and amino acid T366S, L368A and Y407V mutations in the CH3 domain of the “hole chain”; and additionally comprises amino acid R409D and K370E mutations in the CH3 domain of the “knobs chain” and amino acid D399K and E357K mutations in the CH3 domain of the “hole chain”.

In one embodiment the heterodimerization approach described in WO2013/157953 is used alternatively. In one embodiment the CH3 domain of one heavy chain comprises an amino acid T366K mutation and the CH3 domain of the other heavy chain comprises an amino acid L351D mutation. In a further embodiment the CH3 domain of the one heavy chain further comprises an amino acid L351K mutation. In a further embodiment the CH3 domain of the other heavy chain further comprises an amino acid mutation selected from Y349E, Y349D and L368E (in one embodiment L368E).

In one embodiment the heterodimerization approach described in WO2012/058768 is used alternatively. In one embodiment the CH3 domain of one heavy chain comprises amino acid L35 1Y and Y407A mutations and the CH3 domain of the other heavy chain comprises amino acid T366A and K409F mutations. In a further embodiment the CH3 domain of the other heavy chain further comprises an amino acid mutation at position T411, D399, 5400, F405, N390 or K392. In one embodiment said amino acid mutation is selected from the group consisting of

a) T411N, T411R, T411Q, T411K, T411D, T411E and T411W,

b) D399R, D399W, D399Y and D399K,

c) S400E, S400D, S400R and S400K,

d) F4051, F405M, F405T, F405S, F405V and F405W,

e) N390R, N390K and N390D,

f) K392V, K392M, K392R, K392L, K392F and K392E.

In a further embodiment the CH3 domain of one heavy chain comprises amino acid L351Y and Y407A mutations and the CH3 domain of the other heavy chain comprises amino acid T366V and K409F mutations. In a further embodiment the CH3 domain of one heavy chain comprises an amino acid Y407A mutation and the

CH3 domain of the other heavy chain comprises amino acid T366A and K409F mutations. In a further embodiment the CH3 domain of the other heavy chain further comprises amino acid K392E, T411E, D399R and S400R mutations.

In one embodiment the heterodimerization approach described in WO2011/143545 is used alternatively. In one embodiment the amino acid modification according to WO2011/143545 is introduced in the CH3 domain of the heavy chain at a position selected from the group consisting of 368 and 409.

In one embodiment the heterodimerization approach described in WO2011/090762 which also uses the knob-into-hole technology described above is used alternatively. In one embodiment the CH3 domain of one heavy chain comprises an amino acid T366W mutation and the CH3 domain of the other heavy chain comprises an amino acid Y407A mutation. In one embodiment the CH3 domain of one heavy chain comprises an amino acid T366Y mutation and the CH3 domain of the other heavy chain comprises an amino acid Y407T mutation.

In one embodiment the multispecific antibody is of IgG2 isotype and the heterodimerization approach described in WO2010/129304 is used alternatively.

In one embodiment the heterodimerization approach described in WO2009/089004 is used alternatively. In one embodiment the CH3 domain of one heavy chain comprises an amino acid substitution of K392 or N392 with a negatively-charged amino acid (in one embodiment glutamic acid (E) or aspartic acid (D); in a further embodiment a K392D or N392D mutation) and the CH3 domain of the other heavy chain comprises an amino acid substitution of D399, E356, D356, or E357 with a positively-charged amino acid (in one embodiment Lysine (K) or arginine (R), in a further embodiment a D399K, E356K, D356K or E357K substitution; and in an even further embodiment a D399K or E356K mutation). In a further embodiment the CH3 domain of the one heavy chain further comprises an amino acid substitution of K409 or R409 with a negatively-charged amino acid (in one embodiment glutamic acid (E) or aspartic acid (D); in a further embodiment a K409D or R409D mutation). In a further embodiment the CH3 domain of the one heavy chain further or alternatively comprises an amino acid substitution of K439 and/or K370 with a negatively-charged amino acid (in one embodiment glutamic acid (E) or aspartic acid (D)).

In one embodiment the heterodimerization approach described in WO2007/147901 is used alternatively. In one embodiment the CH3 domain of one heavy chain comprises amino acid K253E, D282K and K322D mutations and the CH3 domain of the other heavy chain comprises amino acid D239K, E240K and K292D mutations.

In one embodiment the heterodimerization approach described in WO2007/110205 is used alternatively.
In one embodiment the bispecific antibody which binds to human vascular endothelial growth factor (VEGF) and to human angiopoietin-2 (ANG-2) is a bispecific anti-VEGF/ANG2 antibody comprising a first antigen-binding site that specifically binds to human VEGF and a second antigen-binding site that specifically binds to human ANG-2, wherein
   i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
   ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14, and wherein
   iii) the bispecific antibody comprises a constant heavy chain region of human IgG1 subclass comprising the mutations I253A, H310A, and H435A and the mutations L234A, L235A and P329G (numberings according to EU Index of Kabat; and wherein
   iv) in the constant heavy chain region a T366W mutation is comprised in one CH3 domain and T366S, L368A, Y407V mutations are comprised the other CH3 domain (numberings according to EU Index of Kabat).
In one embodiment the bispecific antibody which binds to human vascular endothelial growth factor (VEGF) and to human angiopoietin-2 (ANG-2) is a bispecific anti-VEGF/ANG2 antibody comprising a first antigen-binding site that specifically binds to human VEGF and a second antigen-binding site that specifically binds to human ANG-2, wherein
   i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
   ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14, and wherein
   iii) the bispecific antibody comprises a constant heavy chain region of human IgG1 subclass comprising the mutations I253A, H310A, and H435A and the mutations L234A, L235A and P329G (numberings according to EU Index of Kabat; and wherein
   iv) in the constant heavy chain region a S354C and T366W mutations are comprised in one CH3 domain and Y349C, T366S, L368A and Y407V mutations are comprised the other CH3 domain (numberings according to EU Index of Kabat).

In one embodiment such bispecific anti-VEGF/ANG2 antibody is bivalent.
In one embodiment such bispecific anti-VEGF/ANG2 antibody is characterized in that
   i) said first antigen-binding site specifically binding to VEGF comprises as heavy chain variable domain VH an amino acid sequence of SEQ ID NO: 7, and as light chain variable domain VL an amino acid sequence of SEQ ID NO: 8, and
   ii) said second antigen-binding site specifically binding to ANG-2 comprises as heavy chain variable domain VH an amino acid sequence of SEQ ID NO: 15, and as light chain variable domain VL an amino acid sequence of SEQ ID NO: 16.

In one aspect of the invention such bispecific, bivalent antibody according to the invention is characterized in comprising
a) the heavy chain and the light chain of a first full length antibody that specifically binds to VEGF;
b) the modified heavy chain and modified light chain of a second full length antibody that specifically binds to ANG-2, wherein the constant domains CL and CH1 are replaced by each other.

The term “VEGF” as used herein refers to human vascular endothelial growth factor (VEGF/VEGF-A,) the 165-amino acid human vascular endothelial cell growth factor (amino acid 27-191 of precursor sequence of human VEGF165: SEQ ID NO: 24; amino acids 1-26 represent the signal peptide), and related 121, 189, and 206 vascular endothelial cell growth factor isoforms, as described by Leung, D. W., et al., Science 246 (1989) 1306-9; Houck et al., Mol. Endocrin. 5 (1991) 1806 -1814; Keck, P. J., et al., Science 246 (1989) 1309-12 and Connolly, D. T., et al., J. Biol. Chem. 264 (1989) 20017-24; together with the naturally occurring allelic and processed forms of those growth factors. VEGF is involved in the regulation of normal and abnormal angiogenesis and neovascularization associated with tumors and intraocular disorders (Ferrara, N., et al., Endocr. Rev. 18 (1997) 4-25; Berkman, R. A., et al., J. Clin. Invest. 91 (1993) 153-159; Brown, L. F., et al., Human Pathol. 26 (1995) 86-91; Brown, L. F., et al., Cancer Res. 53 (1993) 4727-4735; Mattern, J., et al., Brit. J. Cancer. 73 (1996) 931-934; and Dvorak, H. F., et al., Am. J. Pathol. 146 (1995) 1029-1039). VEGF is a homodimeric glycoprotein that has been isolated from several sources and includes several isoforms. VEGF shows highly specific mitogenic activity for endothelial cells. A VEGF antagonist/inhibitor inhibits binding of VEGF to its receptor VEGFR. Known VEGF antagonist/inhibitors include bispecific anti-VEGF/ANG2 antibodies as described in WO2014/009465.

The term “ANG-2” as used herein refers to human angiopoietin-2 (ANG-2) (alternatively abbreviated with ANGPT2 or ANG2) (SEQ ID NO: 25) which is described e.g. in Maisonpierre, P. C., et al, Science 277 (1997) 55-60 and Cheung, A. H., et al., Genomics 48 (1998) 389-91. The angiopoietins-1 (SEQ ID NO: 26) and −2 were discovered as ligands for the Ties, a family of tyrosine kinases that is selectively expressed within the vascular endothelium (Yancopoulos, G. D., et al., Nature 407 (2000) 242-48). There are now four definitive members of the angiopoietin family. Angiopoietin-3 and -4 (Ang-3 and Ang-4) may represent widely diverged counterparts of the same gene locus in mouse and man (Kim, I., et al., FEBS Let, 443 (1999) 353-56; Kim, I., et al., J Biol Chem 274 (1999) 26523-28). ANG-1 and ANG-2 were originally identified in tissue culture experiments as agonist and antagonist, respectively (see for ANG-1: Davis, S., et al., Cell 87 (1996) 1161-69; and for ANG-2: Maisonpierre, P. C., et al., Science 277 (1997) 55-60). All of the known angiopoietins bind primarily to its receptor TIE2 (SEQ ID NO: 27), and both Ang-1 and -2 bind to TIE2 with an affinity of 3 nM (Kd) (Maisonpierre, P. C., et al., Science 277 (1997) 55-60). An ANG2 antagonist/inhibitor inhibits binding of ANG2 to its receptor TIE2. Known ANG2 antagonist/inhibitors include bispecific anti-VEGF/ANG2 antibodies as described in WO2014/009465.

An antigen-binding sites of the bispecific antibody of the invention contain six complementarity determining regions (CDRs) which contribute in varying degrees to the affinity of the binding site for antigen. There are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences.

The antibodies of the invention comprise immunoglobulin constant regions derived from human origin of one or more immunoglobulin classes, wherein such immunoglobulin classes include IgG, IgM, IgA, IgD, and IgE classes and, in the case of IgG and IgA, their subclasses, especially IgG1 and IgG4.

The terms “monoclonal antibody” or “monoclonal antibody composition” as used herein refer to a preparation of antibody molecules of a single amino acid composition.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” encompassed by the present invention are those in which the constant region has been modified or changed from that of the original antibody to generate the properties according to the invention, especially in regard to C1q binding and/or Fc receptor (FcR) binding. Such chimeric antibodies are also referred to as “class-switched antibodies”. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art. See, e.g., Morrison, S. L., et al., Proc. Natl. Acad. Sci. USA 81 (1984) 6851-6855; U.S. Pat. Nos. 5,202,238 and 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See, e.g., Riechmann, L., et al., Nature 332 (1988) 323-327; and Neuberger, M. S., et al., Nature 314 (1985) 268-270. Particularly preferred CDRs correspond to those representing sequences recognizing the antigens noted above for chimeric antibodies. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Brueggemann, M., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole, A., et al. and Boerner, P., et al. are also available for the preparation of human monoclonal antibodies (Cole, A., et al., Monoclonal Antibodies and Cancer Therapy, Liss, A. L., p. 77 (1985); and Boerner, P., et al., J. Immunol. 147 (1991) 86-95). As already mentioned for chimeric and humanized antibodies according to the invention the term “human antibody” as used herein also comprises such antibodies which are modified in the constant region to generate the properties according to the invention, especially in regard to C1q binding and/or FcR binding, e.g. by “class switching” i.e. change or mutation of Fc parts (e.g. from IgG1 to IgG4 and/or IgG1/IgG4 mutation).

The term “recombinant antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant antibodies have variable and constant regions in a rearranged form. The recombinant antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The “variable domain” (variable domain of a light chain (VL), variable domain of a heavy chain (VH) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a n-sheet conformation and the CDRs may form loops connecting the n-sheet structure. The CDRs in each chain are held in their three-dimensional structure by the framework regions and form together with the CDRs from the other chain the antigen binding site. The antibody heavy and light chain CDR3 regions play a particularly important role in the binding specificity/affinity of the antibodies according to the invention and therefore provide a further object of the invention.

The terms “hypervariable region” or “antigen-binding portion of an antibody” when used herein refer to the amino acid residues of an antibody which are responsible for antigen-binding. The hypervariable region comprises amino acid residues from the “complementarity determining regions” or “CDRs”. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. CDRs on each chain are separated by such framework amino acids. Especially, CDR3 of the heavy chain is the region which contributes most to antigen binding. CDR and FR regions are determined according to the standard definition of Kabat, E. A., et al., Sequences of Proteins of Immunological Interest, 5th ed., Public Health Service, National Institutes of Health, Bethesda, Md. (1991).

The term “full length antibody” denotes an antibody consisting of two “full length antibody heavy chains” and two “full length antibody light chains”. A “full length antibody heavy chain” is a polypeptide consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH1-HR-CH2-CH3; and optionally an antibody heavy chain constant domain 4 (CH4) in case of an antibody of the subclass IgE. Preferably the “full length antibody heavy chain” is a polypeptide consisting in N-terminal to C-terminal direction of VH, CH1, HR, CH2 and CH3. A “full length antibody light chain” is a polypeptide consisting in N-terminal to C-terminal direction of an antibody light chain variable domain (VL), and an antibody light chain constant domain (CL), abbreviated as VL-CL. The antibody light chain constant domain (CL) can be κ (kappa) or λ (lambda). The two full length antibody chains are linked together via inter-polypeptide disulfide bonds between the CL domain and the CH1 domain and between the hinge regions of the full length antibody heavy chains. Examples of typical full length antibodies are natural antibodies like IgG (e.g. IgG 1 and IgG2), IgM, IgA, IgD, and IgE. The full length antibodies according to the invention can be from a single species e.g. human, or they can be chimerized or humanized antibodies. The full length antibodies according to the invention comprise two antigen binding sites each formed by a pair of VH and VL, which both specifically bind to the same antigen. The C-terminus of the heavy or light chain of said full length antibody denotes the last amino acid at the C-terminus of said heavy or light chain. The N-terminus of the heavy or light chain of said full length antibody denotes the last amino acid at the N-terminus of said heavy or light chain.

The term “constant region” as used within the current applications denotes the sum of the domains of an antibody other than the variable region. The constant region is not involved directly in binding of an antigen, but exhibits various effector functions. Depending on the amino acid sequence of the constant region of their heavy chains, antibodies are divided in the classes: IgA, IgD, IgE, IgG and IgM, and several of these may be further divided into subclasses, such as IgG1, IgG2, IgG3, and IgG4, IgA1 and IgA2. The heavy chain constant regions that correspond to the different classes of antibodies are called α, δ, ε, γ, and μ, respectively. The light chain constant regions which can be found in all five antibody classes are called κ (kappa) and λ (lambda).

The terms “constant region derived from human origin” or “human constant region” as used in the current application denotes a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., et al., Sequences of Proteins of Immunological Interest, 5th ed., Public Health Service, National Institutes of Health, Bethesda, Md. (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Within the application for the numbering of positions and mutations the EU numbering system (EU Index) according to Kabat, E. A., et al., Sequences of Proteins of Immunological Interest, 5th ed., Public Health Service, National Institutes of Health, Bethesda, Md. (1991) is used and referred to as “numbering according to EU Index of Kabat”.

In one embodiment the bispecific antibodies according to the invention have a constant region of human IgG1 subclass (derived from human IgG1 subclass). However, the C-terminal lysine (Lys447), or the C-terminal glycine (Gly446) and the C-terminal lysine (Lys447), of the Fc region may or may not be present.

In one embodiment the bispecific antibody as described herein is of IgG1 isotype/subclass and comprises a constant heavy chain domain of SEQ ID NO: 23 or the constant parts of the heavy chain amino acid sequence of SEQ ID NO: 17 and of the heavy chain amino acid sequence of SEQ ID NO: 18. In one embodiment additionally the C-terminal glycine (Gly446) is present. In one embodiment additionally the C-terminal glycine (Gly446) and the C-terminal lysine (Lys447) is present.

Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, Md. (1991), NIH Publication 91-3242.

In one embodiment the bispecific antibody according to the invention is of human IgG1 subclass with mutations L234A (Leu235A1a), L235A (Leu234A1a) and P329G (Pro329Gly). Such antibody has a reduced FcR binding (especially they show no more binding to FcRgammaI, FcRgammaII and FcRgammaIII). This especially useful to reduce potential side effects like e.g. thrombosis (Meyer, T., et al., J. Thromb. Haemost. 7 (2009) 171-81).

While Pro329Ala mutation which was described already removes only two third of the FcgammaRIIIa sandwich interaction, the Pro329Gly in the antibodies according to the invention fully imparts binding of the Fc part to FcgammaRIII. This is especially useful as the binding to FcgammaRIII is involved in ADCC (antibody-dependent cellular toxicity) which leads to cell death, which may be helpful in the treatment of cancer diseases, but which can cause serious side effect in the antibody based treatment of other vascular or immunological diseases. So the antibodies according to the invention of IgG1 subclass with mutations L234A, L235A and P329G and IgG4 subclass with mutations S228P, L235E and P329G are especially useful, as they both show no more binding to FcRgammaI, FcRgammaII and FcRgammaIII.

An “effective amount” of an agent, e.g., a pharmaceutical formulation or bispecific anti-VEGF/ANG2 antibody, refers to an amount effective, at dosages and for periods of time necessary, to achieve the desired therapeutic or prophylactic result.

In one embodiment of the invention the bispecific antibody, medicament or pharmaceutical formulation as described herein is administered via intravitreal application, e.g. via intravitreal injection (is administered “intravitreally”). This can be performed in accordance with standard procedures known in the art. See, e.g., Ritter et al., J. Clin. Invest. 116 (2006) 3266-76; Russelakis-Carneiro et al., Neuropathol. Appl. Neurobiol. 25 (1999) 196-206; and Wray et al., Arch. Neurol. 33 (1976) 183-5.

In some embodiments, therapeutic kits of the invention can contain one or more doses of the bispecific antibody described present in a medicament or pharmaceutical formulation, a suitable device for intravitreal injection of the medicament or pharmaceutical formulation, and an instruction detailing suitable subjects and protocols for carrying out the injection. In these embodiments, the medicament or pharmaceutical formulation are typically administered to the subject in need of treatment via intravitreal injection. This can be performed in accordance with standard procedures known in the art. See, e.g., Ritter et al., J. Clin. Invest. 116 (2006) 3266-76; Russelakis-Cameiro et al., Neuropathol. Appl. Neurobiol. 25 (1999) 196-206; and Wray et al., Arch. Neurol. 33 (1976) 183-5.

Regardless of the route of administration selected, the bispecific antibody as described herein is formulated into pharmaceutically acceptable dosage forms by conventional methods known to those of skill in the art.
One embodiment is the method of treatment or the bispecific antibody (medicament or pharmaceutical formulation) for use in the treatment of ocular vascular diseases according to any one of the preceding claims wherein the antibody is administered according to determinations of a software tool.
Another embodiment is method of providing a personalized dosing schedule according to a personalized treatment interval (PTI) for the treatment of a patient suffering from nAMD, the method comprising:
   receiving, at a computing system, patient data comprising a patient's CST and best-corrected visual acuity (BCVA); and optionally, the information on the assessment of new macular hemorrhages;
   using the computing system, extending, reducing, or maintaining a dosing interval based on the received patient data compared with respective reference CST and BCVA; and
   generating a PTI from the dosing interval based on the criteria as described herein for the different ocular vascular diseases like nAMD, DME or macular edema secondary to RVO.
Another embodiment is a computer device/computing system for use/for implementation of such a method.

Description of the Amino Acid Sequences

| | | |
|---|---|---|
| SEQ ID NO: | 1 | heavy chain CDR3H, <VEGF>ranibizumab |
| SEQ ID NO: | 2 | heavy chain CDR2H, <VEGF>ranibizumab |
| SEQ ID NO: | 3 | heavy chain CDR1H, <VEGF>ranibizumab |
| SEQ ID NO: | 4 | light chain CDR3L, <VEGF>ranibizumab |
| SEQ ID NO: | 5 | light chain CDR2L, <VEGF>ranibizumab |
| SEQ ID NO: | 6 | light chain CDR1L, <VEGF>ranibizumab |
| SEQ ID NO: | 7 | heavy chain variable domain VH, <VEGF>ranibizumab |
| SEQ ID NO: | 8 | light chain variable domain VL, <VEGF>ranibizumab |
| SEQ ID NO: | 9 | heavy chain CDR3H, <ANG-2> Ang2i_LC10 variant |
| SEQ ID NO: | 10 | heavy chain CDR2H, <ANG-2> Ang2i_LC10 variant |
| SEQ ID NO: | 11 | heavy chain CDR1H, <ANG-2> Ang2i_LC10 variant |
| SEQ ID NO: | 12 | light chain CDR3L, <ANG-2> Ang2i_LC10 variant |
| SEQ ID NO: | 13 | light chain CDR2L, <ANG-2> Ang2i_LC10 variant |
| SEQ ID NO: | 14 | light chain CDR1L, <ANG-2> Ang2i_LC10 variant |
| SEQ ID NO: | 15 | heavy chain variable domain VH, <ANG-2> |
| | | Ang2i_LC10 variant |
| SEQ ID NO: | 16 | light chain variable domain VL, <ANG-2> |
| | | Ang2i_LC10 variant |
| SEQ ID NO: | 17 | Heavy chain 1 of <VEGF-ANG-2> CrossMAb IgG1 |
| | | with AAA mutations and P329G LALA mutations |
| | | (VEGFang2-0016) |
| SEQ ID NO: | 18 | Heavy chain 2 of <VEGF-ANG-2> CrossMAb IgG1 |
| | | with AAA mutations and P329G LALA mutations |
| | | (VEGFang2-0016) |
| SEQ ID NO: | 19 | Light chain 1 of <VEGF-ANG-2> CrossMAb IgG1 |
| | | with AAA mutations and P329G LALA mutations |
| | | (VEGFang2-0016) |
| SEQ ID NO: | 20 | Light chain 2 of <VEGF-ANG-2> CrossMAb IgG1 |
| | | with AAA mutations and P329G LALA mutations |
| | | (VEGFang2-0016) |
| SEQ ID NO: | 21 | kappa light chain constant region |
| SEQ ID NO: | 22 | lambda light chain constant region |
| SEQ ID NO: | 23 | heavy chain constant region derived from human IgG1 |
| SEQ ID NO: | 24 | Human vascular endothelial growth factor (VEGF); |
| | | precursor sequence of human VEGF165 |
| SEQ ID NO: | 25 | Human angiopoietin-2 (ANG-2) |
| SEQ ID NO: | 26 | Human angiopoietin-1 (ANG-1) |
| SEQ ID NO: | 27 | Human Tie-2 receptor |

In the following, embodiments of the invention are listed:
1. A bispecific antibody which binds to human vascular endothelial growth factor (VEGF) and to human angiopoietin-2 (ANG-2) (or a medicament or pharmaceutical formulation comprising the bispecific antibody, or the bispecific antibody for use in the preparation of a medicament), for use in the treatment of an ocular vascular diseases selected from neovascular AMD (nAMD) and diabetic macular edema (DME) or of patients suffering from an ocular vascular diseases selected from neovascular AMD (nAMD) and diabetic macular edema (DME), wherein the treatment includes a personalized treatment interval (PTI).
2. The bispecific antibody (for use) (medicament or pharmaceutical formulation) according to embodimentl, for use in the treatment of neovascular age-related macular degeneration (nAMD) or of patients suffering from nAMD.
3. The bispecific antibody (for use) (medicament or pharmaceutical formulation) according to embodiment 2, wherein the treatment includes a personalized treatment interval, wherein
   a) patients are treated first 4 times with the bispecific VEGF/ANG2 antibody at an every 4 weeks (Q4W) dosing interval;
   b) at Weeks 20 and 24 the disease activity is assessed wherein the disease activity is determined if one of the following criteria are met:
      i) increase of >50 μm in central subfield thickness (CST) compared with the average CST value over the previous two scheduled visits which are Weeks 12 and 16 for the Week 20 assessment, and Weeks 16 and 20 for the Week 24 assessment, or
      ii) increase ≥75 μm in CST compared with the lowest CST value recorded at either of the previous two scheduled visits;
      iii) decrease ≥5 letters in best-corrected visual acuity (BCVA) compared with average BCVA value over the previous two scheduled visits, owing to nAMD disease activity,
      iv) decrease ≥10 letters in BCVA compared with the highest BCVA value recorded at either of the previous two scheduled visits, owing to nAMD disease activity, or
      v) presence of new macular hemorrhage, owing to nAMD activity
   c) then patients
      i) patients who meet the disease activity criteria at Week20 will be treated at a Q8W dosing interval from week 20 onward (with the first Q8W dosing at Week20);
      ii) patients who meet the disease activity criteria at Week24 will be treated at a Q12W dosing interval from week 24 onward (with the first Q12W dosing at Week24); and
      iii) patients who do not meet disease activity criteria at Week20 and Week24 will be treated at a Q16W dosing interval from week 28 onward (with the first Q16W dosing at Week28).
4. The bispecific antibody (for use) (medicament or pharmaceutical formulation) according to embodiment 3, wherein the personalized treatment interval will be extended, reduced, or maintained after week 60 wherein the
   a) interval is extended by 4 weeks (to a maximum of Q16W) if all of the following criteria are met:
      i) stable CST compared with the average of the last 2 study drug dosing visits where stability is defined as a change of CST of less than 30 μm and no increase ≥50 μm in CST compared with the lowest on-study drug dosing visit measurement,
      ii) no decrease ≥5 letters in BCVA compared with the average from the last two study drug dosing visits, and no decrease ≥10 letters in BCVA compared with the highest on-study drug dosing visit measurement,
      iii) no new macular hemorrhage;
   b) interval
      is reduced (to a minimum Q8W) by 4 weeks if one of the following criteria is met,
      or
      is reduced to an 8-week interval if two or more of the following criteria are met or one criterion includes new macular hemorrhage:
      i) increase of ≥50 μm in CST compared with the average from the last two dosing visits or of ≥75 μm compared with the lowest dosing visit measurement,
      ii) decrease of ≥5 letters in BCVA compared with average of last two dosing visits or decrease ≥10 letters in BCVA compared with the highest dosing visit measurement,
      iii) new macular hemorrhage.
5. The bispecific antibody (for use) (medicament or pharmaceutical formulation) according to embodiment 1, for use in the treatment of diabetic macular edema

(DME) or of patients suffering from DME.
6. The bispecific antibody (for use) (medicament or pharmaceutical formulation) according to embodiment 5, wherein the treatment includes a personalized treatment interval (PTI), wherein
   a) patients are treated first with the bispecific VEGF/ANG2 antibody at an every 4 weeks (Q4W) dosing interval until the central subfield thickness (CST) meets a predefined reference CST threshold (of CST <325 μm for Spectralis spectral domain-central subfield thickness SD-OCT, or <315 μm for Cirrus SD-OCT or Topcon SD-OCT) (as measured at week 12 or later);
   b) then the dosing interval is increased by 4 weeks to an initial Q8W dosing interval;
   c) from this point forward, the dosing interval is extended, reduced, or maintained based on assessments made at the dosing visits which are based on the relative change of the CST and best-corrected visual acuity (BCVA) compared with the respective reference CST and BCVA;
   wherein the
   i) interval is extended by 4 weeks,
      if the CST value is increased or decreased by ≤10% without an associated ≥10-letter BCVA decrease;
   ii) interval will be maintained:
      if the CST is decreased by >10%, or
      the CST value is increased or decreased by ≤10% with an associated ≥10-letter BCVA decrease, or
      the CST value is increased between >10% and ≤20% without an associated ≥5-letter BCVA decrease;
   iii) interval is reduced by 4 weeks
      if the CST value is increased between >10% and ≤20% with an associated ≥5 to <10-letter BCVA decrease; or
      the CST value is increased by >20% without an associated ≥10-letter BCVA decrease;
   iv) interval is reduced by 8 weeks if the CST value is increased by >10% with an associated ≥10-letter BCVA decrease;
   wherein the respective reference central subfield thickness (CST) is the CST value when the initial CST threshold criteria are met and the reference CST is adjusted if CST decreases by >10% from the previous reference CST for two consecutive dosing visits and the values obtained are within 30 μm so that the CST value obtained at the latter visit will serve as the new reference CST; and
   wherein the reference best-corrected visual acuity (BCVA) is the mean of the three best BCVA scores obtained at any prior dosing visit.
7. The bispecific antibody (for use) (medicament or pharmaceutical formulation) according to the embodiment 6, wherein the dosing interval can by adjusted by 4-week increments to a maximum of every 16 weeks (Q16W) and a minimum of Q4W.
8. A bispecific antibody which binds to human vascular endothelial growth factor (VEGF) and to human angiopoietin-2 (ANG-2), for use in the treatment of an ocular vascular disease selected from macular edema secondary to central retinal vein occlusion, secondary to hemiretinal vein occlusion or secondary to branch vein occlusion, or of patients suffering from an ocular vascular disease selected from macular edema secondary to central retinal vein occlusion, secondary to hemiretinal vein occlusion or secondary to branch vein occlusion, wherein the treatment includes a personalized treatment interval (PTI), wherein
   a) patients are treated first with the bispecific VEGF/ANG2 antibody at an every 4 weeks (Q4W) dosing interval from Day 1 through Week 20
   b) from Week 24, patients receive the bispecific VEGF/ANG2 antibody at a frequency of Q4W until the central subfield thickness (CST) meets a predefined reference CST threshold (of CST <325 μm for Spectralis spectral domain-central subfield thickness SD-OCT, or <315 μm for Cirrus SD-OCT or Topcon SD-OCT) (as measured at week 24 or later);
   c) from this point forward, the dosing interval is extended, reduced, or maintained based on assessments made at the dosing visits which are based on the relative change of the CST and best-corrected visual acuity (BCVA) compared with the respective reference CST and BCVA;
      wherein the
         i) interval is extended by 4 weeks
         if the CST value is increased or decreased by ≤10% without an associated ≥10-letter BCVA decrease; or
         ii) interval is maintained if any of the following criteria are met:
         if the CST value is decreased by >10%; or
         if the CST value is decreased ≤10% with an associated ≥10-letter BCVA decrease; or
         if the CST value is increased between >10% and ≤20% without an associated >5-letter BCVA decrease;
         iii) interval is reduced by 4 weeks if any of the following criteria are met:
         if the CST value is increased between >10% and ≤20% with an associated ≥5-to <10-letter BCVA decrease, or
         if the CST value is increased by >20% without an associated ≥10-letter BCVA decrease, or
         if the CST value is increased by ≤10% with an associated BCVA decrease of ≥10-letters;
         iv) interval is reduced to Q4W
         if the CST value is increased by >10% with an associated ≥10-letter BCVA decrease,
         wherein the respective reference central subfield thickness (CST) is the CST value when the initial CST threshold criteria are met and the reference CST is adjusted if CST decreases by >10% from the previous reference CST for two consecutive dosing visits and the values obtained are within 30 μm so that the CST value obtained at the latter visit will serve as the new reference CST; and
         wherein the reference best-corrected visual acuity (BCVA) is the mean of the three best BCVA scores obtained at any prior dosing visit.
9. The bispecific antibody (for use) (medicament or pharmaceutical formulation) according to the embodiment 8, wherein the dosing interval can by adjusted to a maximum of every 16 weeks (Q16W) and a minimum of Q4W.
10. The bispecific antibody (for use) (medicament or pharmaceutical formulation) according to any one of embodiments 1 to 9, wherein the bispecific antibody which binds to human VEGF and to human ANG2 is a bispecific, bivalent anti-VEGF/ANG2 antibody comprising a first antigen-binding site that specifically binds to human VEGF and a second antigen-binding site that specifically binds to human ANG-2, wherein
   i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
   ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14,
   and wherein
   iii) the bispecific antibody comprises a constant heavy chain region of human IgG1 subclass comprising the mutations I253A, H310A, and H435A and the mutations L234A, L235A and P329G (numberings according to EU Index of Kabat).
11. The bispecific antibody (for use) (medicament or pharmaceutical formulation) according to embodiment 10, wherein
   i) said first antigen-binding site specifically binding to VEGF comprises as heavy chain variable domain VH an amino acid sequence of SEQ ID NO: 7, and as light chain variable domain VL an amino acid sequence of SEQ ID NO: 8, and
   ii) said second antigen-binding site specifically binding to ANG-2 comprises as heavy chain variable domain VH an amino acid sequence of SEQ ID NO: 15, and as light chain variable domain VL an amino acid sequence of SEQ ID NO: 16.
12. The bispecific antibody (for use) (medicament or pharmaceutical formulation) according to any one of embodiments 1 to 9, wherein the bispecific antibody which binds to human VEGF and human ANG2 comprises the amino acid sequences of SEQ ID NO: 17, of SEQ ID NO: 18, of SEQ ID NO: 19, and of SEQ ID NO: 20.
13. The bispecific antibody (for use) (medicament or pharmaceutical formulation) according to any one of embodiments 1 to 9, wherein the bispecific antibody is faricimab.
14. The bispecific antibody (for use) (medicament or pharmaceutical formulation) according to any one of embodiments 10 to 13, wherein the bispecific antibody is administered in a dose of about 5 to 7 mg (at each treatment).
15. The bispecific antibody (for use) (medicament or pharmaceutical formulation) according to any one of embodiments 8 to 13, wherein the bispecific antibody is administered in a dose of about 6 mg (at each treatment).
16. The bispecific antibody (for use) (medicament or pharmaceutical formulation) according to any one of embodiments 14 to 15, wherein the bispecific antibody is administered at a concentration of about 120 mg/ml.
17. The bispecific antibody (for use) (medicament or pharmaceutical formulation) according to any one of the preceding embodiments wherein patients suffering from an ocular vascular disease have not been previously treated with anti-VEGF treatment.
18. The bispecific antibody (for use) (medicament or pharmaceutical formulation) according to any one of the preceding embodiments wherein patients suffering from an ocular vascular disease have been previously treated with anti-VEGF treatment.
19. The bispecific antibody for use (medicament or pharmaceutical formulation) according to any one of the preceding embodiments wherein the antibody is administered according to determinations of a software tool.
20. A method of providing a personalized dosing schedule according to a personalized treatment interval (PTI) for the treatment of a patient suffering from nAMD, the method comprising:
   receiving, at a computing system, patient data comprising a patient's CST and best-corrected visual acuity (BCVA) and optionally the information on the assessment of new macular hemorrhages; and
   using the computing system, extending, reducing, or maintaining a dosing interval based on the received patient data compared with respective reference CST and BCVA; and
   generating a PTI from the dosing interval, wherein the
   a) interval is extended by 4 weeks (to a maximum of Q16W) if all of the following criteria are met:
      i) stable CST compared with the average of the last 2 study drug dosing visits where stability is defined as a change of CST of less than 30 μm and no increase ≥50 μm in CST compared with the lowest on-study drug dosing visit measurement,
      ii) no decrease ≥5 letters in BCVA compared with the average from the last two study drug dosing visits, and no decrease >10 letters in BCVA compared with the highest on-study drug dosing visit measurement,
      iii) no new macular hemorrhage
   b) interval
      is reduced (to a minimum Q8W) by 4 weeks if one of the following criteria is met,
      or
      is reduced to an 8-week interval if two or more of the following criteria are met or one criterion includes new macular hemorrhage:
      i) increase of ≥50 μm in CST compared with the average from the last two dosing visits or of ≥75 μm compared with the lowest dosing visit measurement;
      ii) decrease of ≥5 letters in BCVA compared with average of last two dosing visits or decrease ≥10 letters in BCVA compared with the highest dosing visit measurement;
      iii) new macular hemorrhage.
21. A method of providing a personalized dosing schedule according to a personalized treatment interval (PTI) for the treatment of a patient suffering from DME, the method comprising:
   receiving, at a computing system, patient data comprising a patient's CST and best-corrected visual acuity (BCVA); and
   using the computing system, extending, reducing, or maintaining a dosing interval based on the received patient data compared with respective reference CST and BCVA; and
   generating a PTI from the dosing interval, wherein the
   i) interval is extended by 4 weeks,
      if the CST value is increased or decreased by ≤10% without an associated ≥10-letter BCVA decrease;
   ii) interval will be maintained:
      if the CST is decreased by >10%, or
      the CST value is increased or decreased by ≤10% with an associated ≥10-letter BCVA decrease, or
      the CST value is increased between >10% and ≤20% without an associated ≥5-letter BCVA decrease;
   iii) interval is reduced by 4 weeks
      if the CST value is increased between >10% and ≤20% with an associated ≥5 to <10-letter BCVA decrease; or
      the CST value is increased by >20% without an associated ≥10-letter BCVA decrease;
   iv) interval is reduced by 8 weeks if the CST value is increased by >10% with an associated >10-letter BCVA decrease.
22. A method of providing a personalized dosing schedule according to a personalized treatment interval (PTI) for the treatment of a patient suffering from an ocular vascular disease selected from macular edema secondary to central retinal vein occlusion, secondary to hemiretinal vein occlusion or secondary to branch vein occlusion, the method comprising:
   receiving, at a computing system, patient data comprising a patient's CST and best-corrected visual acuity (BCVA); and
   using the computing system, extending, reducing, or maintaining a dosing interval based on the received patient data compared with respective reference CST and BCVA; and
   generating a PTI from the dosing interval, wherein the
      i) interval is extended by 4 weeks
      if the CST value is increased or decreased by ≤10% without an associated ≤10-letter BCVA decrease; or
      ii) interval is maintained if any of the following criteria are met:
      if the CST value is decreased by >10%; or
      if the CST value is decreased ≤10% with an associated ≥10-letter BCVA decrease; or
      if the CST value is increased between >10% and ≤20% without an associated ≥5-letter BCVA decrease;
      iii) interval is reduced by 4 weeks if any of the following criteria are met:
      if the CST value is increased between >10% and ≤20% with an associated ≥5-to <10-letter BCVA decrease, or
      if the CST value is increased by >20% without an associated ≥10-letter BCVA decrease, or
      if the CST value is increased by ≤10% with an associated BCVA decrease of ≥10-letters;
      iv) interval is reduced to Q4W
      if the CST value is increased by >10% with an associated ≥10-letter BCVA decrease.
23. The method of any one of embodiments 20, 21 or 22, further comprising:
   receiving, at the computing system, updated patient data;
   using the computing system, continually updating or maintaining the dosing interval based on the updated patient data; and
   generating a visualization, user interface, or notification based on the updated or maintained dosing interval.
24. Use of a personalized dosing schedule according to a personalized treatment interval (PTI) (for the treatment of nAMD), wherein a computing system generates the PTI by:
   receiving, at a computing system, patient data comprising a patient's CST and best-corrected visual acuity (BCVA) and optionally the information on the assessment of new macular hemorrhages; and
   extending, reducing, or maintaining a dosing interval based on the received patient data compared with respective reference CST and BCVA;
      wherein the
   a) interval is extended by 4 weeks (to a maximum of Q16W) if all of the following criteria are met:
      i) stable CST compared with the average of the last 2 study drug dosing visits where stability is defined as a change of CST of less than 30 μm and no increase ≥50 μm in CST compared with the lowest on-study drug dosing visit measurement,
      ii) no decrease ≥5 letters in BCVA compared with the average from the last two study drug dosing visits, and no decrease ≥10 letters in BCVA compared with the highest on-study drug dosing visit measurement,
      iii) no new macular hemorrhage
   b) interval
      is reduced (to a minimum Q8W) by 4 weeks if one of the following criteria is met,
      or
      is reduced to an 8-week interval if two or more of the following criteria are met or one criterion includes new macular hemorrhage:
      i) increase of ≥50 μm in CST compared with the average from the last two dosing visits or of ≥75 μm compared with the lowest dosing visit measurement;
      ii) decrease of ≥5 letters in BCVA compared with average of last two dosing visits or decrease ≥10 letters in BCVA compared with the highest dosing visit measurement;
      iii) new macular hemorrhage.
25. Use of a personalized dosing schedule according to a personalized treatment interval (PTI) (for the treatment of DME), wherein a computing system generates the PTI by:
   receiving patient data comprising a patient's CST and best-corrected visual acuity (BCVA); and
   extending, reducing, or maintaining a dosing interval based on the received patient data compared with respective reference CST and BCVA;
      wherein the
   i) interval is extended by 4 weeks,
      if the CST value is increased or decreased by ≤10% without an associated ≥10-letter BCVA decrease;
   ii) interval will be maintained:
      if the CST is decreased by >10%, or
      the CST value is increased or decreased by ≤10% with an associated ≥10-letter BCVA decrease, or
      the CST value is increased between >10% and ≤20% without an associated ≥5-letter BCVA decrease;
   iii) interval is reduced by 4 weeks
      if the CST value is increased between >10% and ≤20% with an associated ≥5 to <10-letter BCVA decrease; or
      the CST value is increased by >20% without an associated ≥10-letter BCVA decrease;
   iv) interval is reduced by 8 weeks if the CST value is increased by >10% with an associated ≥10-letter BCVA decrease
26. Use of a personalized dosing schedule according to a personalized treatment interval (PTI) (for the treatment of macular edema secondary to central retinal vein occlusion, secondary to hemiretinal vein occlusion or secondary to branch vein occlusion), wherein a computing system generates the PTI by:
   receiving patient data comprising a patient's CST and best-corrected visual acuity (BCVA); and
   extending, reducing, or maintaining a dosing interval based on the received patient data compared with respective reference CST and BCVA;
      wherein the
         i) interval is extended by 4 weeks
         if the CST value is increased or decreased by ≤10% without an associated ≥10-letter BCVA decrease; or
         ii) interval is maintained if any of the following criteria are met:
         if the CST value is decreased by >10%; or
         if the CST value is decreased ≤10% with an associated ≥10-letter BCVA decrease; or
         if the CST value is increased between >10% and ≤20% without an associated ≥5-letter BCVA decrease;
         iii) interval is reduced by 4 weeks if any of the following criteria are met:
         if the CST value is increased between >10% and ≤20% with an associated ≥5-to <10-letter BCVA decrease, or
         if the CST value is increased by >20% without an associated ≥10-letter BCVA decrease, or
         if the CST value is increased by ≤10% with an associated BCVA decrease of ≥10-letters;
         iv) interval is reduced to Q4W
         if the CST value is increased by >10% with an associated ≥10-letter BCVA decrease.

In the following, embodiments of the invention are listed:
1. A method of treating patients suffering from an ocular vascular disease selected from neovascular AMD (nAMD) and diabetic macular edema (DME) the method comprising administering to the patient an effective amount of a bispecific antibody which binds to human vascular endothelial growth factor (VEGF) and to human angiopoietin-2 (ANG-2), wherein the treatment includes a personalized treatment interval (PTI).
2. The method according to embodiment 1, wherein the ocular vascular disease is neovascular age-related macular degeneration (nAMD).
3. The method according to embodiment 2, wherein the treatment includes a personalized treatment interval, wherein
   a) patients are treated first 4 times with the bispecific VEGF/ANG2 antibody at an every 4 weeks (Q4W) dosing interval;
   b) at Weeks 20 and 24 the disease activity is assessed wherein the disease activity is determined if one of the following criteria are met:
      i) increase of >50 μm in central subfield thickness (CST) compared with the average CST value over the previous two scheduled visits which are Weeks 12 and 16 for the Week 20 assessment, and Weeks 16 and 20 for the Week 24 assessment, or
      ii) increase ≥75 μm in CST compared with the lowest CST value recorded at either of the previous two scheduled visits;
      iii) decrease ≥5 letters in best-corrected visual acuity (BCVA) compared with average BCVA value over the previous two scheduled visits, owing to nAMD disease activity,
      iv) decrease ≥10 letters in BCVA compared with the highest BCVA value recorded at either of the previous two scheduled visits, owing to nAMD disease activity, or
      v) presence of new macular hemorrhage, owing to nAMD activity
   c) then patients
      i) patients who meet the disease activity criteria at Week20 will be treated at a Q8W dosing interval from week 20 onward (with the first Q8W dosing at Week20);
      ii) patients who meet the disease activity criteria at Week24 will be treated at a Q12W dosing interval from week 24 onward (with the first Q12W dosing at Week24); and
      iii) patients who do not meet disease activity criteria at Week20 and Week24 will be treated at a Q16W dosing interval from week 28 onward (with the first Q16W dosing at Week28).
4. The method according to embodiment 3, wherein the personalized treatment interval will be extended, reduced, or maintained after week 60 wherein the
   a) interval is extended by 4 weeks (to a maximum of Q16W) if all of the following criteria are met:
      i) stable CST compared with the average of the last 2 study drug dosing visits where stability is defined as a change of CST of less than 30 μm and no increase ≥50 μm in CST compared with the lowest on-study drug dosing visit measurement,
      ii) no decrease ≥5 letters in BCVA compared with the average from the last two study drug dosing visits, and no decrease ≥10 letters in BCVA compared with the highest on-study drug dosing visit measurement,
      iii) no new macular hemorrhage;
   b) interval
      is reduced (to a minimum Q8W) by 4 weeks if one of the following criteria is met,
      or
      is reduced to an 8-week interval if two or more of the following criteria are met or one criterion includes new macular hemorrhage:
      i) increase of ≥50 μm in CST compared with the average from the last two dosing visits or of ≥75 μm compared with the lowest dosing visit measurement,
      ii) decrease of ≥5 letters in BCVA compared with average of last two dosing visits or decrease ≥10 letters in BCVA compared with the highest dosing visit measurement,
      iii) new macular hemorrhage.
5. The method according to embodiment 1, for use in the treatment of diabetic macular edema (DME) or of patients suffering from DME.
6. The method according to embodiment 5, wherein the treatment includes a personalized treatment interval (PTI), wherein
   a) patients are treated first with the bispecific VEGF/ANG2 antibody at an every 4 weeks (Q4W) dosing interval until the central subfield thickness (CST) meets a predefined reference CST threshold (of CST <325 μm for Spectralis spectral domain-central subfield thickness SD-OCT, or <315 μm for Cirrus SD-OCT or Topcon SD-OCT) (as measured at week 12 or later);
   b) then the dosing interval is increased by 4 weeks to an initial Q8W dosing interval;
   c) from this point forward, the dosing interval is extended, reduced, or maintained based on assessments made at the dosing visits. which are based on the relative change of the CST and best-corrected visual acuity (BCVA) compared with the respective reference CST and BCVA;
   wherein the
   i) interval is extended by 4 weeks,
      if the CST value is increased or decreased by ≤10% without an associated ≥10-letter BCVA decrease;
   ii) interval will be maintained:
      if the CST is decreased by >10%, or
      the CST value is increased or decreased by ≤10% with an associated ≥10-letter BCVA decrease, or
      the CST value is increased between >10% and ≤20% without an associated ≥5-letter BCVA decrease;
   iii) interval is reduced by 4 weeks
      if the CST value is increased between >10% and ≤20% with an associated ≥5 to <10-letter BCVA decrease; or
      the CST value is increased by >20% without an associated ≥10-letter BCVA decrease;
   iv) interval is reduced by 8 weeks if the CST value is increased by >10% with an associated ≥10-letter BCVA decrease;
   wherein the respective reference central subfield thickness (CST) is the CST value when the initial CST threshold criteria are met and the reference CST is adjusted if CST decreases by >10% from the previous reference CST for two consecutive dosing visits and the values obtained are within 30 μm so that the CST value obtained at the latter visit will serve as the new reference CST; and
   wherein the reference best-corrected visual acuity (BCVA) is the mean of the three best BCVA scores obtained at any prior dosing visit.
7. The method according to the embodiment 6, wherein the dosing interval can by adjusted by 4-week increments to a maximum of every 16 weeks (Q16W) and a minimum of Q4W.
8. A method of treating patients suffering from an ocular vascular disease selected from macular edema secondary to central retinal vein occlusion, secondary to hemiretinal vein occlusion or secondary to branch vein occlusion the method comprising administering to the patient an effective amount of a bispecific antibody which binds to human vascular endothelial growth factor (VEGF) and to human angiopoietin-2 (ANG-2), wherein the treatment includes a personalized treatment interval (PTI), wherein
   a) patients are treated first with the bispecific VEGF/ANG2 antibody at an every 4 weeks (Q4W) dosing interval from Day 1 through Week 20
   b) from Week 24, patients receive the bispecific VEGF/ANG2 antibody at a frequency of Q4W until the central subfield thickness (CST) meets a predefined reference CST threshold (of CST <325 μm for Spectralis spectral domain-central subfield thickness SD-OCT, or <315 μm for Cirrus SD-OCT or Topcon SD-OCT) (as measured at week 24 or later);
   c) from this point forward, the dosing interval is extended, reduced, or maintained based on assessments made at the dosing visits which are based on the relative change of the CST and best-corrected visual acuity (BCVA) compared with the respective reference CST and BCVA;
      wherein the
         i) interval is extended by 4 weeks
         if the CST value is increased or decreased by ≤10% without an associated ≥10-letter BCVA decrease; or
         ii) interval is maintained if any of the following criteria are met:
         if the CST value is decreased by >10%; or
         if the CST value is decreased ≤10% with an associated ≥10-letter BCVA decrease; or
         if the CST value is increased between >10% and ≤20% without an associated ≥5-letter BCVA decrease;
         iii) interval is reduced by 4 weeks if any of the following criteria are met:
         if the CST value is increased between >10% and ≤20% with an associated ≥5-to <10-letter BCVA decrease, or
         if the CST value is increased by >20% without an associated ≥10-letter BCVA decrease, or
         if the CST value is increased by ≤10% with an associated BCVA decrease of ≥10-letters;
         iv) interval is reduced to Q4W
         if the CST value is increased by >10% with an associated ≥10-letter BCVA decrease,
         wherein the respective reference central subfield thickness (CST) is the CST value when the initial CST threshold criteria are met and the reference CST is adjusted if CST decreases by >10% from the previous reference CST for two consecutive dosing visits and the values obtained are within 30 μm so that the CST value obtained at the latter visit will serve as the new reference CST; and
         wherein the reference best-corrected visual acuity (BCVA) is the mean of the three best BCVA scores obtained at any prior dosing visit.
9. The method according to the embodiment 8, wherein the dosing interval can by adjusted to a maximum of every 16 weeks (Q16W) and a minimum of Q4W.
10. The method according to any one of embodiments 1 to 9, wherein the bispecific antibody which binds to human VEGF and to human ANG2 is a bispecific, bivalent anti-VEGF/ANG2 antibody comprising a first antigen-binding site that specifically binds to human VEGF and a second antigen-binding site that specifically binds to human ANG-2, wherein
   i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
   ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14,
   and wherein
   iii) the bispecific antibody comprises a constant heavy chain region of human IgG1 subclass comprising the mutations I253A, H310A, and

H435A and the mutations L234A, L235A and P329G (numberings according to EU Index of Kabat).
11. The method according to embodiment 10, wherein
   i) said first antigen-binding site specifically binding to VEGF comprises as heavy chain variable domain VH an amino acid sequence of SEQ ID NO: 7, and as light chain variable domain VL an amino acid sequence of SEQ ID NO: 8, and
   ii) said second antigen-binding site specifically binding to ANG-2 comprises as heavy chain variable domain VH an amino acid sequence of SEQ ID NO: 15, and as light chain variable domain VL an amino acid sequence of SEQ ID NO: 16.
12. The method according to any one of embodiments 1 to 9, wherein the bispecific antibody which binds to human VEGF and human ANG2 comprises the amino acid sequences of SEQ ID NO: 17, of SEQ ID NO: 18, of SEQ ID NO: 19, and of SEQ ID NO: 20.
13. The method according to any one of embodiments 1 to 9, wherein the bispecific antibody is faricimab.
14. The method according to any one of embodiments 10 to 13, wherein the bispecific antibody is administered in a dose of about 5 to 7 mg (at each treatment).
15. The method according to any one of embodiments 10 to 13, wherein the bispecific antibody is administered in a dose of about 6 mg (at each treatment).
16. The method according to any one of embodiments 14 to 15, wherein the bispecific antibody is administered at a concentration of about 120 mg/ml.
17. The method according to any one of the preceding embodiments wherein patients suffering from an ocular vascular disease have not been previously treated with anti-VEGF treatment.
18. The method according to any one of the preceding embodiments wherein patients suffering from an ocular vascular disease have been previously treated with anti-VEGF treatment.
19. The method according to any one of the preceding embodiments wherein the antibody is administered according to determinations of a software tool.
20. A method of providing a personalized dosing schedule according to a personalized treatment interval (PTI) for the treatment of a patient suffering from nAMD, the method comprising:
   receiving, at a computing system, patient data comprising a patient's CST and best-corrected visual acuity (BCVA) and optionally the information on the assessment of new macular hemorrhages; and
   using the computing system, extending, reducing, or maintaining a dosing interval based on the received patient data compared with respective reference CST and BCVA; and
   generating a PTI from the dosing interval, wherein the
   a) interval is extended by 4 weeks (to a maximum of Q16W) if all of the following criteria are met:
      i) stable CST compared with the average of the last 2 study drug dosing visits where stability is defined as a change of CST of less than 30 μm and no increase ≥50 μm in CST compared with the lowest on-study drug dosing visit measurement,
      ii) no decrease ≥5 letters in BCVA compared with the average from the last two study drug dosing visits, and no decrease ≥10 letters in BCVA compared with the highest on-study drug dosing visit measurement,
      iii) no new macular hemorrhage
   b) interval
      is reduced (to a minimum Q8W) by 4 weeks if one of the following criteria is met,
      or
      is reduced to an 8-week interval if two or more of the following criteria are met or one criterion includes new macular hemorrhage:
      i) increase of ≥50 μm in CST compared with the average from the last two dosing visits or of ≥75 μm compared with the lowest dosing visit measurement;
      ii) decrease of ≥5 letters in BCVA compared with average of last two dosing visits or decrease ≥10 letters in BCVA compared with the highest dosing visit measurement;
      iii) new macular hemorrhage.
21. A method of providing a personalized dosing schedule according to a personalized treatment interval (PTI) for the treatment of a patient suffering from DME, the method comprising:
   receiving, at a computing system, patient data comprising a patient's CST and best-corrected visual acuity (BCVA); and
   using the computing system, extending, reducing, or maintaining a dosing interval based on the received patient data compared with respective reference CST and BCVA; and
   generating a PTI from the dosing interval, wherein the
   i) interval is extended by 4 weeks,
      if the CST value is increased or decreased by ≤10% without an associated ≥10-letter BCVA decrease;
   ii) interval will be maintained:
      if the CST is decreased by >10%, or
      the CST value is increased or decreased by ≤10% with an associated ≥10-letter BCVA decrease, or
      the CST value is increased between >10% and ≤20% without an associated ≥5-letter BCVA decrease;
   iii) interval is reduced by 4 weeks
      if the CST value is increased between >10% and ≤20% with an associated ≥5 to <10-letter BCVA decrease; or
      the CST value is increased by >20% without an associated ≥10-letter BCVA decrease;
   iv) interval is reduced by 8 weeks if the CST value is increased by >10% with an associated ≥10-letter BCVA decrease.
22. A method of providing a personalized dosing schedule according to a personalized treatment interval (PTI) for the treatment of a patient suffering from an ocular vascular disease selected from macular edema secondary to central retinal vein occlusion, secondary to hemiretinal vein occlusion or secondary to branch vein occlusion, the method comprising:
   receiving, at a computing system, patient data comprising a patient's CST and best-corrected visual acuity (BCVA); and
   using the computing system, extending, reducing, or maintaining a dosing interval based on the received patient data compared with respective reference CST and BCVA; and
   generating a PTI from the dosing interval, wherein the
      i) interval is extended by 4 weeks
      if the CST value is increased or decreased by ≤10% without an associated ≥10-letter BCVA decrease; or
      ii) interval is maintained if any of the following criteria are met:
      if the CST value is decreased by >10%; or
      if the CST value is decreased ≤10% with an associated ≥10-letter BCVA decrease; or
      if the CST value is increased between >10% and ≤20% without an associated ≥5-letter BCVA decrease;
      iii) interval is reduced by 4 weeks if any of the following criteria are met:
      if the CST value is increased between >10% and ≤20% with an associated ≥5-to <10-letter BCVA decrease, or
      if the CST value is increased by >20% without an associated ≥10-letter BCVA decrease, or
      if the CST value is increased by ≤10% with an associated BCVA decrease of ≥10-letters;
      iv) interval is reduced to Q4W
      if the CST value is increased by >10% with an associated ≥10-letter BCVA decrease.
23. The method of any one of embodiments 20, 21 or 22, further comprising:
   receiving, at the computing system, updated patient data;
   using the computing system, continually updating or maintaining the dosing interval based on the updated patient data; and
   generating a visualization, user interface, or notification based on the updated or maintained dosing interval.
24. Use of a personalized dosing schedule according to a personalized treatment interval (PTI) (for the treatment of nAMD), wherein a computing system generates the PTI by:
   receiving, at a computing system, patient data comprising a patient's CST and best-corrected visual acuity (BCVA) and optionally the information on the assessment of new macular hemorrhages; and
   extending, reducing, or maintaining a dosing interval based on the received patient data compared with respective reference CST and BCVA; wherein the
   a) interval is extended by 4 weeks (to a maximum of Q16W) if all of the following criteria are met:
      i) stable CST compared with the average of the last 2 study drug dosing visits where stability is defined as a change of CST of less than 30 μm and no increase ≥50 μm in CST compared with the lowest on-study drug dosing visit measurement,
      ii) no decrease ≥5 letters in BCVA compared with the average from the last two study drug dosing visits, and no decrease ≥10 letters in BCVA compared with the highest on-study drug dosing visit measurement,
      iii) no new macular hemorrhage
   b) interval
      is reduced (to a minimum Q8W) by 4 weeks if one of the following criteria is met,
      or
      is reduced to an 8-week interval if two or more of the following criteria are met or one criterion includes new macular hemorrhage:
      i) increase of ≥50 μm in CST compared with the average from the last two dosing visits or of ≥75 μm compared with the lowest dosing visit measurement;
      ii) decrease of ≥5 letters in BCVA compared with average of last two dosing visits or decrease ≥10 letters in BCVA compared with the highest dosing visit measurement;
      iii) new macular hemorrhage.
25. Use of a personalized dosing schedule according to a personalized treatment interval (PTI) (for the treatment of DME), wherein a computing system generates the PTI by:
   receiving patient data comprising a patient's CST and best-corrected visual acuity (BCVA); and
   extending, reducing, or maintaining a dosing interval based on the received patient data compared with respective reference CST and BCVA;
      wherein the
   i) interval is extended by 4 weeks,
      if the CST value is increased or decreased by ≤10% without an associated ≥10-letter BCVA decrease;
   ii) interval will be maintained:
      if the CST is decreased by >10%, or
      the CST value is increased or decreased by ≤10% with an associated ≥10-letter BCVA decrease, or
      the CST value is increased between >10% and ≤20% without an associated ≥5-letter BCVA decrease;
   iii) interval is reduced by 4 weeks
      if the CST value is increased between >10% and ≤20% with an associated ≥5 to <10-letter BCVA decrease; or
      the CST value is increased by >20% without an associated ≥10-letter BCVA decrease;
   iv) interval is reduced by 8 weeks if the CST value is increased by >10% with an associated ≥10-letter BCVA decrease
26. Use of a personalized dosing schedule according to a personalized treatment interval (PTI) (for the treatment of macular edema secondary to central retinal vein occlusion, secondary to hemiretinal vein occlusion or secondary to branch vein occlusion), wherein a computing system generates the PTI by:
   receiving patient data comprising a patient's CST and best-corrected visual acuity (BCVA); and
   extending, reducing, or maintaining a dosing interval based on the received patient data compared with respective reference CST and BCVA;
      wherein the
         i) interval is extended by 4 weeks
         if the CST value is increased or decreased by ≤10% without an associated ≥10-letter BCVA decrease; or
         ii) interval is maintained if any of the following criteria are met:
         if the CST value is decreased by >10%; or
         if the CST value is decreased ≤10% with an associated ≥10-letter BCVA decrease; or
         if the CST value is increased between >10% and ≤20% without an associated ≥5-letter BCVA decrease;
         iii) interval is reduced by 4 weeks if any of the following criteria are met:
         if the CST value is increased between >10% and ≤20% with an associated ≥5-to <10-letter BCVA decrease, or
         if the CST value is increased by >20% without an associated ≥10-letter BCVA decrease, or
         if the CST value is increased by ≤10% with an associated BCVA decrease of ≥10-letters;
         iv) interval is reduced to Q4W
         if the CST value is increased by >10% with an associated ≥10-letter BCVA decrease.

### EXAMPLES

Treatment of Patient Suffering from Vascular Eye Diseases with a Bispecific Antibody that Binds to Human VEGF and Human ANG2

### Example 1

Efficacy and Durability of Treatment of Patients Suffering from Neovascular Age-Related Macular Degeneration (nAMD) Using a Personalized Treatment Interval

In an earlier Phase II, 52-week study to investigate, inter alia, the efficacy of RO6867461 (faricimab) administered at 12- and 16-week intervals in treatment-naive patients with nAMD some potential of longer durability (potential longer time to retreatment) over all patients involved could be seen. Three arms were studied

Arm A (Q12W): 6 mg RO6867461 intravitreally (IVT) every 4 weeks up to Week 12 (4 injections), followed by 6 mg RO6867461 IVT every 12 weeks up to Week 48 (injections at Weeks 24, 36, and 48; 3 injections)

Arm B (Q16W): 6 mg RO6867461 IVT every 4 weeks up to Week 12 (4 injections), followed by 6 mg RO6867461 IVT every 16 weeks up to Week 48 (injections at Weeks 28 and 44; 2 injections)

Arm C (comparator arm): 0.5 mg ranibizumab IVT every 4 weeks for 48 weeks (13 injections) Only one eye will be chosen as the study eye.

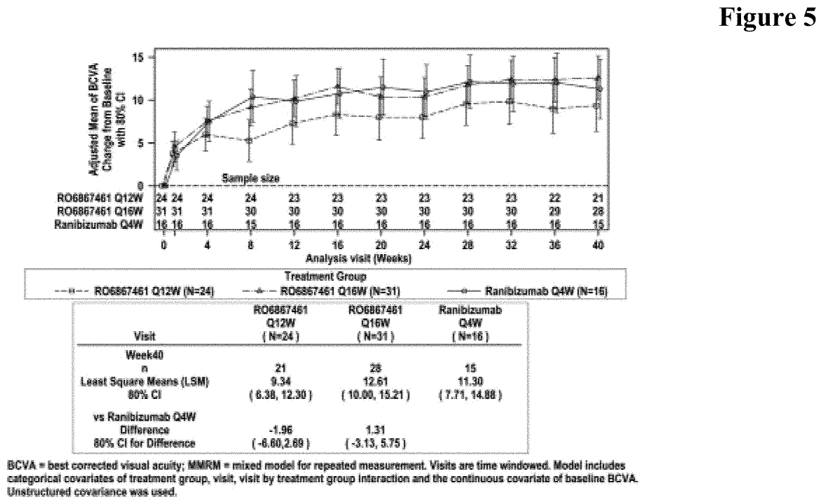

Results with respect to BCVA are shown in FIG. 5. FIG. 5 shows the BCVA gains from baseline of patients with neovascular age-related macular degeneration (nAMD) comparing the bispecific anti-VEGF/ANG2 antibody RO6867461 (faricimab) at 12- and 16-week intervals and ranibizumab (Lucentis®) ((administered intravitreally with a 0.3 mg dose)) at 4-week intervals.

A follow-up Phase III study was initiated which will now evaluate the efficacy, safety, durability, and pharmacokinetics of the 6-mg dose of faricimab administered at up to 16-week intervals (with a specific personalized treatment interval (PTI) schedule) compared with aflibercept monotherapy Q8W in patients with CNV secondary to AMD, also known as nAMD. Faricimab will be administered at a concentration of about 120 mg/ml.

Specific objectives and corresponding endpoints for the study are outlined in Table 1.

TABLE 1
Objectives and Corresponding Endpoints

| Primary Efficacy Objective | Corresponding Endpoint |
|---|---|
| To evaluate the efficacy of IVT | Change from baseline in BCVA (as measured |
| injections of the 6-mg dose of faricimab | on the ETDRS chart at a starting distance of |
| on BCVA outcomes compared with | 4 meters) based on an average at Weeks 40, |
| aflibercept | 44, and 48 |
| Secondary Efficacy Objectives | Corresponding Endpoints |
| To evaluate the efficacy of faricimab on | Change from baseline in BCVA over time |
| additional BCVA outcomes | Proportion of patients gaining ≥15, ≥10, ≥5, |
| | or ≥0 letters in BCVA from baseline over |
| | time |
| | Proportion of patients avoiding loss of ≥15, |
| | ≥10, ≥5, or >0 letters in BCVA from |
| | baseline over time |
| | Proportion of patients with BCVA Snellen |
| | equivalent of 20/40 or better over time |
| | Proportion of patients gaining ≥15 letters or |
| | achieving BCVA of ≥84 letters over time |
| | Proportion of patients with BCVA Snellen |
| | equivalent of 20/200 or worse over time |
| To evaluate the frequency of study drug | Proportion of patients on a Q8W, Q12W, and |
| administration | Q16W treatment interval at Weeks 48, 60, |
| | and 112 |
| | Number of study drug injections received |
| | through Weeks 48, 60, and 112 |
| To evaluate the efficacy of faricimab on | Change from baseline in CST based on an |
| anatomic outcome measures using OCT | average at Weeks 40, 44, and 48 |
| | Change from baseline in CST over time |
| | Proportion of patients with absence of |
| | intraretinal fluid over time |
| | Proportion of patients with absence of |
| | subretinal fluid over time |
| | Proportion of patients with absence of |
| | intraretinal and subretinal fluid over time |
| | Proportion of patients with absence of |
| | intraretinal cysts over time |
| | Proportion of patients with absence of pigment |
| | epithelial detachment over time |
| To evaluate the efficacy of faricimab on | Change from baseline in total area of CNV |
| anatomic outcome measures using FFA | lesion at Week 48 and Week 112 |
| | Change from baseline in total area of leakage |
| | at Week 48 and Week 112 |
| Safety Objective | Corresponding Endpoints |
| To evaluate the ocular and non-ocular | Incidence and severity of ocular adverse |
| safety and tolerability of faricimab | events |
| | Incidence and severity of non-ocular |
| | adverse events |
| Exploratory Efficacy Objectives | Corresponding Endpoint |
| To evaluate the efficacy of faricimab on | Change from baseline in NEI VFQ-25 |
| patient-reported vision-related functioning | composite score over time |
| and quality of life using the NEI VFQ-25 | |
| Pharmacokinetic Objectives | Corresponding Endpoints |
| To characterize the systemic | Plasma concentration of faricimab over time |
| pharmacokinetics of faricimab | |
| Immunogenicity Objectives | Corresponding Endpoints |
| To evaluate the immune response to | Presence of ADAs during the study relative |
| faricimab | to the presence of ADAs at baseline |
| To evaluate potential effects of ADAs | Relationship between ADA status and |
| | efficacy, safety, or PK endpoints |
| Exploratory Pharmacokinetic, | |
| Pharmacodynamic, and Biomarker | |
| Objectives | Corresponding Endpoints |
| To evaluate potential relationships | Relationship between selected covariates |
| between selected covariates and exposure | and plasma or aqueous humor (optional) |
| to faricimab | concentration or PK parameters for |
| | faricimab |
| To evaluate the drug concentration | Relationship between pharmacokinetics of |
| (exposure)-effect relationship for free | faricimab and concentration of free VEGF- |
| VEGF-A and Ang-2 | A and Ang-2 in aqueous humor (optional), |
| To characterize the aqueous humor | plasma, and/or vitreous (optional) over time |
| (optional) and vitreous (optional) | Aqueous humor (optional) and vitreous |
| pharmacokinetics of faricimab | (optional) concentration of faricimab |
| | over time |
| To explore concentration-effect | Pharmacokinetics of faricimab and the |
| relationship for visual acuity and other | change in BCVA or other endpoints |
| endpoints (e.g., anatomical markers) | (e.g., anatomical markers) over time |
| Primary Efficacy Objective | Corresponding Endpoint |
| To evaluate the efficacy of IVT | Change from baseline in BCVA (as measured |
| injections of the 6-mg dose of faricimab | on the ETDRS chart at a starting distance of |
| on BCVA outcomes compared with | 4 meters) based on an average at Weeks 40, |
| aflibercept | 44, and 48 |
| Secondary Efficacy Objectives | Corresponding Endpoints |
| To evaluate the efficacy of faricimab on | Change from baseline in BCVA over time |
| additional BCVA outcomes | Proportion of patients gaining ≥15, ≥10, ≥5, |
| | or ≥0 letters in BCVA from baseline over |
| | time |
| | Proportion of patients avoiding loss of ≥15, |
| | ≥10, ≥5, or >0 letters in BCVA from |
| | baseline over time |
| | Proportion of patients with BCVA Snellen |
| | equivalent of 20/40 or better over time |
| | Proportion of patients gaining ≥15 letters or |
| | achieving BCVA of ≥84 letters over time |
| | Proportion of patients with BCVA Snellen |
| | equivalent of 20/200 or worse over time |
| To evaluate the frequency of study drug | Proportion of patients on a Q8W, Q12W, and |
| administration | Q16W treatment interval at Weeks 48, 60, |
| | and 112 |
| | Number of study drug injections received |
| | through Weeks 48, 60, and 112 |
| To evaluate the efficacy of faricimab on | Change from baseline in CST based on an |
| anatomic outcome measures using OCT | average at Weeks 40, 44, and 48 |
| | Change from baseline in CST over time |
| | Proportion of patients with absence of |
| | intraretinal fluid over time |
| | Proportion of patients with absence of |
| | subretinal fluid over time |
| | Proportion of patients with absence of |
| | intraretinal and subretinal fluid over time |
| | Proportion of patients with absence of |
| | intraretinal cysts over time |
| | Proportion of patients with absence of pigment |
| | epithelial detachment over time |
| To evaluate the efficacy of faricimab on | Change from baseline in total area of CNV |
| anatomic outcome measures using FFA | lesion at Week 48 and Week 112 |
| | Change from baseline in total area of leakage |
| | at Week 48 and Week 112 |

Patients suffering from neovascular age-related macular degeneration (nAMD) (also called wet age-related macular degeneration (wet AMD) are treated with the bispecific antibody that binds to human VEGF and human ANG2 comprising the amino acid sequences of SEQ ID NO: 17, of SEQ ID NO: 18, of SEQ ID NO: 19, and of SEQ ID NO: 20 (this antibody VEGFang2-0016 and its production is also described in detail in WO2014/009465 which is incorporated by reference). Designations of this bispecific anti-VEGF/ANG2 antibody herein are RO6867461 or RG7716 or VEGFang2-0016, or faricimab. As active comparator in treatment e.g. aflibercept will be used. Patients include anti-VEGF treatment-naïve patients (have not been previously treated with anti-VEGF treatment with e.g. aflibercept and/or ranibizumab and/or other anti-VEGF treatments)). Vials of sterile, colorless to brownish, preservative-free solution of RO6867461 (faricimab) for intravitreal (IVT) administration of 6 mg dose are used.

Study Design

This is a multicenter, randomized, active comparator, double-masked, parallel-group, 112-week study to investigate the efficacy, safety, durability, and pharmacokinetics of faricimab administered at up to 16-week intervals to treatment-naive patients with nAMD.

Approximately 640 patients will be enrolled globally and randomized in a 1:1 ratio to one of two treatment arms:

Arm A (faricimab up to Q16W) (n=320): Patients randomized to Arm A will receive 6 mg of IVT faricimab Q4W up to Week 12 (4 injections). At Week 20, a protocol-defined assessment of disease activity requires patients in Arm A with active disease (for the criteria, see below) to be treated at that visit and to continue with a Q8W dosing regimen of faricimab. A second protocol-defined assessment of disease activity at Week 24 requires patients in Arm A with active disease (excluding those with active disease at Week 20 and therefore receiving a Q8W dosing regimen of faricimab) to be treated at that visit and to continue with a Q12W dosing regimen of faricimab. Patients receiving faricimab who do not have active disease according to the protocol-defined criteria at Week 20 and Week 24 will be treated with a Q16W dosing regimen of faricimab. Patients will continue receiving faricimab on a fixed regimen every 8, 12, or 16 weeks until Week 60 according to the disease activity assessments made at Weeks 20 and 24. From Week 60 (when all patients in Arm A are scheduled to receive faricimab) onward, all patients in Arm A will be treated according to a personalized treatment interval (PTI) dosing regimen (see Table 2 for the PTI dosing criteria) up to Week 108.

Arm B (comparator arm) (Q8W): Patients randomized to Arm B will receive 2 mg of IVT aflibercept Q4W up to Week 8 (3 injections), followed by 2 mg of IVT aflibercept Q8W up to Week 108.

Patients in both treatment arms will complete scheduled study visits Q4W for the entire study duration (112 weeks). A sham procedure will be administered to patients in both treatment arms at study visits with no study treatment administration to maintain masking among treatment arms

FIG. 1 presents an overview of the study design
a At Weeks 20 and 24, patients will undergo a disease activity assessment. Patients with anatomic or functional signs of disease activity at these time points will receive Q8W or Q12W dosing, respectively, rather than Q16W dosing.
b The primary endpoint is the change from baseline in BCVA (as assessed on the ETDRS chart at a starting distance of 4 meters) based on an average at Weeks 40, 44, and 48.
c From Week 60 (when all patients in Arm A are scheduled to receive faricimab) onward, patients in Arm A will be treated according to a PTI dosing regimen (between Q8W and Q16W).
BCVA=best-corrected visual acuity; ETDRS=Early Treatment Diabetic Retinopathy Study; IVT=intravitreal; PTI=personalized treatment interval; Q8W=every 8 weeks; Q12W=every 12 weeks; Q16W=every 16 weeks; W=Week.

Only one eye will be assigned as the study eye. If both eyes are considered eligible (per the inclusion and exclusion criteria), the eye with the worse BCVA, as assessed at screening, will be selected as the study eye (unless based on medical reasons, the investigator deems the other eye to be more appropriate for treatment in the study). There will be a minimum of two investigators per site to fulfill the masking requirements of the study. At least one investigator will be designated as the assessor physician who will be masked to each patient's treatment assignment and who will evaluate ocular assessments. At least one other investigator will be unmasked and will perform study treatments.

The study will consist of a screening period of up to 28 days (Days −28 to −1) in length and an approximately 108-week treatment period, followed by the final study visit at Week 112 (at least 28 days after the last study treatment administration).

Weeks 20 and 24 Disease Activity Criteria

Determination of active disease at Weeks 20 and 24 will be made if any of the following criteria are met:
Increase >50 μm in CST compared with the average CST value over the previous two scheduled visits (Weeks 12 and 16 for the Week 20 assessment and Weeks 16 and 20 for the Week 24 assessment); or
Increase ≥75 μm in CST compared with the lowest CST value recorded at either of the previous two scheduled visits; or
Decrease ≥5 letters in BCVA compared with average BCVA value over the previous two scheduled visits, owing to nAMD disease activity (as determined by the investigator); or
Decrease ≥10 letters in BCVA compared with the highest BCVA value recorded at either of the previous two scheduled visits, owing to nAMD disease activity (as determined by the investigator); or
Presence of new macular hemorrhage (as determined by the investigator), owing to nAMD activity

Additional considerations at Week 24: If there is significant nAMD disease activity at Week 24 that does not meet the criteria above, but which in the opinion of the investigator would otherwise warrant treatment, patients randomized to Arm A will receive 6 mg of faricimab at Week 24 and will continue to receive repeated 12 weekly treatments. Patients randomized to Arm A who meet the disease activity criteria at Week 20 will remain on their Q8W dosing schedule and will not receive treatment at Week 24. Patients randomized to Arm B will remain on their Q8W dosing schedule and will receive aflibercept at Week 24.

Personalized Treatment Interval (PTI) Disease Activity Criteria

Starting at Week 60, when all patients in Arm A are scheduled to receive faricimab, the study drug dosing interval for patients in Arm A will be extended based on assessments made at study drug dosing visits. Study drug dosing interval decisions during the PTI regimen phase for Arm A (and the respective algorithm) are described in Table 2. The decision will be made based on data from visits at which patients received drug. Patients will receive a sham procedure at study visits when they are not receiving treatment with faricimab

TABLE 2
Personalized Treatment Interval Algorithm

| Dosing Interval | Criteria |
|---|---|
| Interval extended by 4 | Stable CST^{a} compared with the average of the last |
| weeks (to a maximum | 2 study drug dosing visits, and no increase ≥50 |
| of Q16W) | μm in CST compared with the lowest on-study |
| | drug dosing visit measurement and |
| | No decrease ≥5 letters in BCVA ^{b} compared with |
| | the average from the last two study drug dosing |
| | visits, and no decrease ≥10 letters in BCVA ^{b} |
| | compared with the highest on-study drug dosing |
| | visit measurement and |
| | No new macular hemorrhage ^{c} |
| Interval reduced (to a | Increase ≥50 μm in CST compared with the |
| minimum Q8W) | average from the last two study drug dosing visits |
| If one of the criteria is | or ≥75 μm compared with the lowest on-study |
| met, the interval will be | drug dosing visit measurement or |
| reduced by 4 weeks. If | Decrease ≥5 letters in BCVA ^{b} compared with |
| two or morecriteria are | average of last two study drug dosing visits or |
| met or one criterion | decrease ≥10 letters in BCVA ^{b} compared with |
| includes new macular | the highest on-study drug dosing visit |
| hemorrhage, the | measurement or |
| interval will be reduced | New macular hemorrhage ^{c} |
| to an 8-week interval. ^{c} | |
| Interval maintained | If extension or reduction criteria have not been |
| | met |

BCVA = best-corrected visual acuity; CST = central subfield thickness;
IRF = intraretinal fluid; nAMD = neovascular age-related macular degeneration;
Q8W = every 8 weeks; Q16W = every 16 weeks; SRF = subretinal fluid.
^{a}Where stability is defined as a change of CST of less than 30 μm
^{b} Change in BCVA should be attributable to nAMD disease activity (as determined by investigator).
^{c} Refers to macular hemorrhage owing to nAMD activity (as determined by investigator).
^{d} Patients whose treatment interval is reduced by 8 weeks from Q16W to Q8W will not be allowed to return to a Q16W interval during the study.

As outlined above in Table 2 the algorithm for the personalized drug treatment interval decision making is based on the relative change of the CST and absolute change in BCVA compared with the reference CST and BCVA, respectively; and in addition on the assessment/finding of new macular hemorrhages.

The algorithm may be implemented by a computing system or device. Such a computing system or device may include a web interface, mobile app, software program, or any clinical decision support tool. For example, patient CST and BCVA scores may be uploaded to a web interface of a personalized dosing interval software tool. Using the uploaded CST and BVCA, the tool may automatically compute and output the timing of a next dose. The tool may further provide dosing schedules or notifications, monitor and generate visualizations of dosing interval changes for a given patient, generate visualizations of dosing interval changes for groups of patients, aggregate received CST and BCVA data to determine trends, or a combination thereof.

Dosing schedules or notifications may include displays of calendar dates of scheduled dosing visit(s) and calendar alerts notifying clinicians or patients of upcoming dosing visits. Visualizations of dosing interval changes may include, for instance, displays of the schematics in Table 2. In one case, a patient's dosing interval adjustment may be shown in one color, and the patient's immediate prior dosing interval adjustment may be shown in another color. To illustrate, a patient may first have their interval extended by 4 weeks, and then have their personalized treatment interval maintained. The tool may generate a visualization of the patient's personalized interval progression by showing the “interval maintained” area of the schematic in Table 2 in green, and the “interval extended by 4 weeks” shown in yellow. Green may reflect the patient's most recent interval computation and yellow may depict results of the patient's immediate prior interval computation. With this visualization, a user of the tool may quickly ascertain that a patient's disease progression is improving, but not so improved that their treatment interval may be extended more.

The tool may further aggregate patient and dosing schedule data and generate visualizations of the aggregated data. Such data analyses may include visualizations of dosing changes for a single patient, similar to the color coding example previously described. Alternately, visualizations may show dosing adjustments across groups of patients. For example, one visualization may show which patients are having interval extensions, and which patients are having interval reductions. This visualization may be organized by various characteristic(s), e.g., patient age, prior treatment, disease state, administered antibody, clinical trial group, etc. The tool may also aggregate and create visualizations from patient CST and BCVA data. The visualizations may show trends in the data to facilitate or generate longitudinal analyses. These visualizations may include alerts, plots, analysis workflow interfaces, or any graphical interface.

The tool may generate dosing schedule outputs or visualizations in response to, or along with ocular assessments and images. In one embodiment, the tool may directly compute patient CST or BVCA. For CST, the tool may receive or directly capture ocular images. The tool may further employ image segmentation, image recognition, or machine learning techniques to compute CST from the ocular images. For BCVA, the tool may administer ocular assessments virtually, prompting and collecting patient user inputs via a user interface or via eye tracking mechanisms. Alternately, the tool may receive, store, and track ocular assessment data. In this way, the tool may track each patient's disease progression and adjust dosing schedules accordingly.

The present embodiments may include a method of providing a personalized dosing schedule according to a personalized treatment interval (PTI) for the treatment of a patient suffering from nAMD, the method comprising: receiving, at a computing system, patient data comprising a patient's CST and best-corrected visual acuity (BCVA); using the computing system, extending, reducing, or maintaining a dosing interval based on the received patient data compared with respective reference CST and BCVA; and generating a PTI from the dosing interval. The exemplary dosing interval is extended by 4 weeks (to a maximum of Q16W) if all of the following criteria are met: i) stable CST compared with the average of the last 2 study drug dosing visits where stability is defined as a change of CST of less than 30 μm and no increase ≥50 μm in CST compared with the lowest on-study drug dosing visit measurement, ii) no decrease ≥5 letters in BCVA compared with the average from the last two study drug dosing visits, and no decrease ≥10 letters in BCVA compared with the highest on-study drug dosing visit measurement, iii) no new macular hemorrhage. The exemplary dosing interval is reduced (to a minimum Q8W) by 4 weeks if one of the following criteria is met, or is reduced to an 8-week interval if two or more of the following criteria are met or one criterion includes new macular hemorrhage: i) increase of ≥50 μm in CST compared with the average from the last two dosing visits or of ≥75 μm compared with the lowest dosing visit measurement, ii) decrease of ≥5 letters in BCVA compared with average of last two dosing visits or decrease ≥10 letters in BCVA compared with the highest dosing visit measurement, iii) new macular hemorrhage.

Such a method of providing a personalized dosing schedule according to a personalized treatment interval (PTI) for the treatment of a patient suffering from nAMD, may further comprise receiving, at the computing system, updated patient data; using the computing system, continually updating or maintaining the dosing interval based on the updated patient data; and generating a visualization, user interface, or notification based on the updated or maintained dosing interval.

The present embodiments also include use of a personalized dosing schedule according to a personalized treatment interval (PTI) (for the treatment of nAMD), wherein a computing system generates the PTI by receiving patient data comprising a patient's CST and best-corrected visual acuity (BCVA); and extending, reducing, or maintaining a dosing interval based on the received patient data compared with respective reference CST and BCVA. The exemplary dosing interval is extended by 4 weeks (to a maximum of Q16W) if all of the following criteria are met: i) stable CST compared with the average of the last 2 study drug dosing visits where stability is defined as a change of CST of less than 30 μm and no increase ≥50 μm in CST compared with the lowest on-study drug dosing visit measurement, ii) no decrease ≥5 letters in BCVA compared with the average from the last two study drug dosing visits, and no decrease ≥10 letters in BCVA compared with the highest on-study drug dosing visit measurement, iii) no new macular hemorrhage. The exemplary dosing interval is reduced (to a minimum Q8W) by 4 weeks if one of the following criteria is met, or is reduced to an 8-week interval if two or more of the following criteria are met or one criterion includes new macular hemorrhage: i) increase of ≥50 μm in CST compared with the average from the last two dosing visits or of ≥75 μm compared with the lowest dosing visit measurement; ii) decrease of ≥5 letters in BCVA compared with average of last two dosing visits or decrease ≥10 letters in BCVA compared with the highest dosing visit measurement; iii) new macular hemorrhage.

Ocular Assessments

Ocular assessments include the following and will be performed at specified time points:
BCVA is measured by using the set of three Precision Vision™ or Lighthouse distance acuity charts (modified ETDRS Charts 1, 2, and R). A VA Manual was provided to the investigators. VA examiner and VA examination room certifications were obtained before any VA examinations were performed. The BCVA examiner is masked to study eye and treatment assignment and will only perform the refraction and BCVA assessment (e.g. Visual Acuity Specification Manual). The BCVA examiner is also masked to the BCVA letter scores of a patient's previous visits and only knew the patient's refraction data from previous visits. The BCVA examiner is not allowed to perform any other tasks involving direct patient care.
Low-luminance BCVA, as assessed on the ETDRS chart at a starting distance of 4 meters Low-Luminance Best-Corrected Visual Acuity Testing. There are the same requirements as the best corrected visual acuity described in Appendix 4; however, low-luminance best-corrected visual acuity will be measured by placing a 2.0 log-unit neutral density filter (Kodak Wratten 2.0 neutral density filter) over the best correction for that eye and having the participant read the normally illuminated Early Treatment Diabetic Retinopathy Study chart.
Pre-treatment IOP (intraocular pressure) measurement of both eyes (performed prior to dilating eyes).
Slitlamp examination (for grading scales for anterior and vitreous cells, see Foster C S, Kothari S, Anesi S D, et al. The Ocular and Uveitis Foundation preferred practice patterns of uveitis management. Sury Opthalmol 61 (2016)1-17).
Dilated binocular indirect high-magnification ophthalmoscopy.
Finger counting test followed by hand motion and light perception tests (when necessary) performed within 15 minutes of post-study treatment in the study eye only by the unmasked treatment administrator.
At study treatment visits, post-treatment IOP measurement in the study eye only within 30 (±15) minutes by qualified personnel assigned to the unmasked role. If there are no safety concerns after 30 (±15) minutes following the study treatment, the patient will be permitted to leave the clinic. If the TOP value is of concern to the treatment administrator, the patient will remain in the clinic and will be managed in accordance with the treatment administrator's clinical judgment. The adverse event will be recorded on the Adverse Event electronic Case Report Form (eCRF) as applicable.

The method of IOP measurement used for a patient must remain consistent throughout the study.

Ocular Imaging

The central reading center(s) (CRC(s)) will provide sites with the central reading center(s) manual and training materials for specified study ocular images. Before any study images are obtained, site personnel, test images, systems, and software (where applicable) will be certified and validated by the reading center(s) as specified in the central reading center manual. All ocular images results will be obtained by trained site personnel at the study sites and forwarded to the central reading center(s) for independent analysis and/or storage.

After randomization, if a patient misses a study visit when ocular images are scheduled, or the images are not taken at the scheduled visit (e.g., due to broken equipment), the images should be obtained at the next scheduled visit the patient attends.

Ocular images include the following:
Color Fundus Photography (CFP) of both eyes. Stereo color fundus photographs will be obtained from both eyes by trained personnel at the study sites. Fundus photography will be performed at the intervals specified in the schedule of activities.
Fundus Fluorescein Angiography (FFA of both eyes (performed after laboratory samples are obtained). Fundus fluorescein angiography will be performed on both eyes at the study sites by trained personnel who are certified by the central reading center. The fundus fluorescein angiograms will be obtained at the intervals specified in the protocol.
Spectral-Domain Optical Coherence Tomography (SD-OCT) or swept-source OCT (SS-OCT) images of both eyes.
Optional OCT-angiography (OCT-A) of both eyes at sites with agreed OCT-A capabilities.
Optional Indocyanine Green Angiography (ICGA) of both eyes at selected sites with agreed ICGA capabilities (performed after laboratory samples are obtained). Indocyanine green angiography (ICGA) will be performed on both eyes by trained personnel who are certified by the central reading center at the intervals specified.

Results

The primary efficacy analyses included all randomized patients, with patients grouped according to the treatment assigned at randomization.

The primary efficacy variable is the BCVA change. The primary efficacy analysis will be performed using e.g. a Mixed Model for Repeated Measurement (MMRM) model.

Best Corrected Visual Acuity

BCVA is measured as described. Primary Efficacy Outcome Measure is shown in a Figure which displays the primary efficacy endpoint: BCVA change from Baseline over Time for patients. The bispecific anti-VEGF/ANG2 antibody RO6867461 (faricimab) comprising the amino acid sequences of SEQ ID NO: 17, of SEQ ID NO: 18, of SEQ ID NO: 19, and of SEQ ID NO: 20 (administered intravitreally with a 6.0 mg as described in Arm A using the personalized treatment interval), is compared e.g. to Arm B (aflibercept (Eylea®) Q8W dosing) according to the study scheme described above.

Central Subfield Thickness (CST) Change from Baseline (Study Eye)

A key secondary endpoint is the change from baseline in CST, central subfield thickness. CST (as well as retinal thickness) is measured via Optical coherence tomography (OCT). Results are shown in a Figure in which the change of CST is shown over time for the bispecific anti-VEGF/ANG2 antibody RO6867461 (faricimab) comprising the amino acid sequences of SEQ ID NO: 17, of SEQ ID NO: 18, of SEQ ID NO: 19, and of SEQ ID NO: 20 (administered intravitreally with a 6.0 mg as described in Arm A using the personalized treatment interval) is compared e.g. to Arm B (aflibercept (Eylea®) Q8W dosing) according to the study scheme described above.

Further outcomes of the ocular assessment and imaging can be displayed accordingly.

### Example 2

Efficacy and Durability of Bispecific Anti-VEGF/ANG2 Treatment of Patients Suffering from Diabetic Macular Edema (DME) Using a Personalized Treatment Interval

In an earlier Phase II, 36-week study in patients with diabetic macular edema (DME) some potential of longer durability (potential longer time to retreatment) over all patients involved could be seen. The three study groups were treated as follows: Arm A: 0.3 mg ranibizumab intravitreal (IVT); Arm B: 1.5 mg RO6867461 (faricimab) IVT; Arm C: 6 mg RO6867461 (faricimab) IVT.

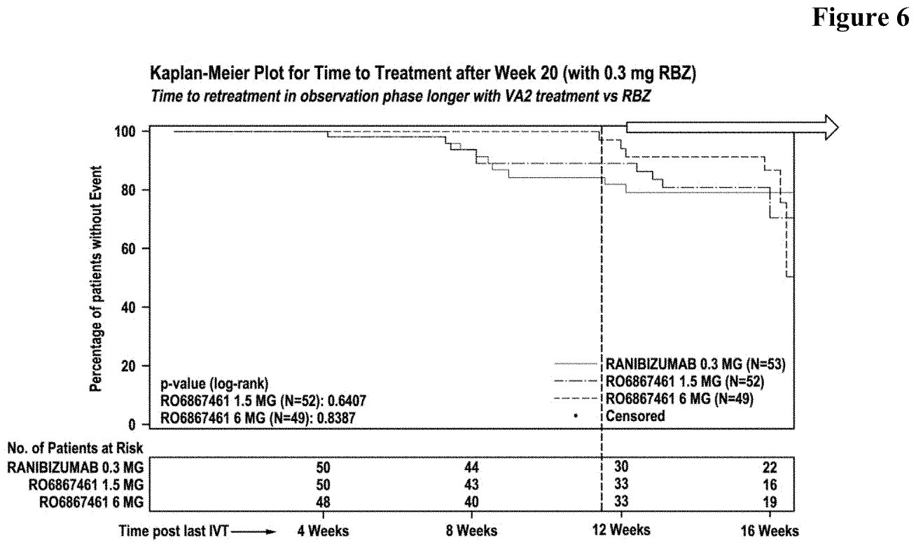

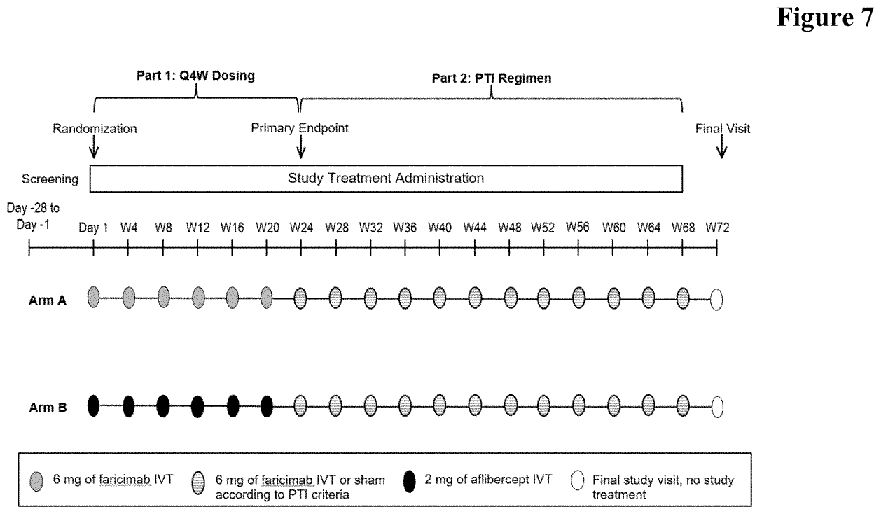

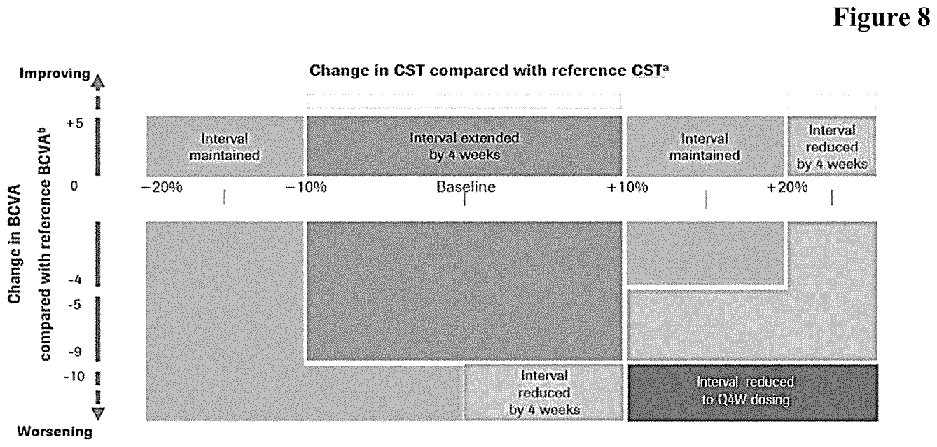

Results with respect to the potential longer time to retreatment for RO6867461 (faricimab, VA2) are shown in FIG. 6. FIG. 6 shows the time to retreatment in DME patients after dosing has discontinued (after 20 weeks or 6 monthly doses=Time post last intravitreal (IVT) administration) based on disease activity assessed by both: BCVA decreased by ≥5 letters and CST increased by ≥50 μm (=patients with an event). The bispecific anti-VEGF/ANG2 antibody RO6867461 (faricimab) (administered intravitreally with a 6.0 mg or 1.5 mg dose), was compared to ranibizumab (Lucentis®) (administered intravitreally with a 0.3 mg dose).

A follow-up Phase III study was initiated which will now evaluate the efficacy, safety, and pharmacokinetics of RO6867461 (faricimab) when administered to patients every 8 weeks (Q8W) and with a personalized treatment interval (PTI) regimen compared with aflibercept (Eylea®) monotherapy in patients with DME. The effect on visual function will be assessed by measuring the change from baseline in best-corrected visual acuity (BCVA) (i.e., the number of ETDRS letters). The effect on retinal anatomy will be evaluated by retinal imaging (spectral-domain optical coherence tomography [SD-OCT], color fundus photographs [CFPs], fundus fluorescein angiography [FFA]), and other imaging modalities to assess both DME and DR outcomes. In addition, safety, patient-reported outcomes (PROs), and the pharmacokinetics of RO6867461 will be assessed.

This study will evaluate the efficacy, safety, and pharmacokinetics of RO6867461 when dosed Q8W and with a PTI regimen compared with aflibercept (Eylea®) monotherapy in patients with DME. Specific objectives and corresponding endpoints for the study are outlined in Table 3.

TABLE 3
Objectives and Corresponding Endpoints

| Primary | |
|---|---|
| Efficacy Objective | Corresponding Endpoint |
| To evaluate the efficacy | Change from baseline in BCVA (as measured |
| of IVT injections of the | on the ETDRS chart at a starting distance of |
| 6-mg dose of faricimab | 4 meters) at 1 year^{a} |
| on BCVA outcomes | |
| Key Secondary | |
| Efficacy Objective | Corresponding Endpoint |
| To evaluate the efficacy | Proportion of patients with a ≥2-step DRS |
| of faricimab on DR | improvement from baseline on the ETDRS |
| severity outcomes | DRSS at Week 52 |
| Secondary Efficacy | |
| Objectives | Corresponding Endpoints |
| To evaluate the efficacy | Change from baseline in BCVA (as measured |
| of faricimab on additional | on the ETDRS chart at a starting distance of |
| BCVA outcomes | 4 meters) over time |
| To evaluate the efficacy | Proportion of patients gaining ≥15, ≥10, ≥5, |
| of faricimab on additional | or ≥0 letters in BCVA from baseline over |
| DR outcomes | time |
| To evaluate faricimab | Proportion of patients avoiding a loss of ≥15, |
| treatment intervals in | ≥10, ≥5, or >0 letters in BCVA from baseline |
| the PTI arm | over time |
| To evaluate the efficacy of | Proportion of patients gaining ≥15 letters or |
| faricimab on anatomical | achieving BCVA of ≥84 letters over time |
| outcome measures using | Proportion of patients with BCVA Snellen |
| SD-OCT | equivalent of 20/40 or better over time |
| To evaluate the efficacy of | Proportion of patients with BCVA Snellen |
| faricimab on patient- | equivalent of 20/200 or worse over time |
| reported vision-related | Proportion of patients with a ≥2-step DRS |
| functioning and quality | improvement from baseline on the ETDRS |
| of life using the NEI | DRSS over time |
| VFQ-25 | Proportion of patients with a ≥3-step DRS |
| | improvement from baseline on the ETDRS |
| | DRSS over time |
| | Proportion of patients who develop new PDR |
| | over time |
| | Proportion of patients in the PTI arm on a |
| | Q4W, Q8W, Q12W, or Q16W treatment |
| | interval at 1 year and 2 years |
| | Treatment intervals in the PTI arm over time |
| | Change from baseline in CST at 1 year ^{a} |
| | Change from baseline in CST over time |
| | Proportion of patients with absence of DME |
| | (CST < 325 μm for Spectralis SD-OCT, or |
| | <315 μm for Cirrus SD-OCT or Topcon SD- |
| | OCT) over time |
| | Proportion of patients with absence of |
| | intraretinal fluid over time |
| | Proportion of patients with absence of |
| | subretinal fluid over time |
| | Proportion of patients with absence of |
| | intraretinal fluid and subretinal fluid over time |
| | Change from baseline in NEI VFQ-25 |
| | composite score over time |
| Safety Objective | Corresponding Endpoints |
| To evaluate the ocular and | Incidence and severity of ocular adverse |
| systemic safety and | events |
| tolerability of faricimab | Incidence and severity of non-ocular |
| | adverse events |
| Exploratory | |
| Efficacy Objectives | Corresponding Endpoints |
| To further evaluate the | Proportion of patients with a ≥2-step or ≥3- |
| efficacy of faricimab on | step DRS worsening from baseline on |
| additional DR outcomes | ETDRS DRSS over time |
| To further evaluate the | Proportion of patients who receive vitrectomy |
| efficacy of faricimab on | or PRP over time during the study |
| anatomical outcome | Change from baseline in the macular and the |
| measures using FFA and/ | total retinal area ^{b} of ischemic non-perfusion |
| or OCT-A ^{c} | (capillary loss) over time |
| To further evaluate the | Change from baseline in vascular leakage in |
| efficacy of faricimab on | the macula and in the total retinal area ^{b} over |
| anatomical outcome | time |
| measures using SD-OCT | Proportion of patients with resolution of |
| To further evaluate the | vascular leakage in the macula and in the |
| efficacy of faricimab | total retinal area ^{b} over time |
| on patient-reported vision- | Change from baseline neurosensory CST |
| related functioning and | over time |
| quality of life using the | Change from baseline in total macular |
| NET VFQ-25 | volume over time |
| | Change from baseline in the NEI VFQ-25 |
| | Near Activities, Distance Activities, and |
| | Driving subscales at 1 year^{a} |
| | Proportion of patients with a 4-point |
| | improvement from baseline in NEI VFQ-25 |
| | composite score at 1 year^{a} |
| Pharmacokinetic | |
| Objective | Corresponding Endpoint |
| To characterize the | Plasma concentration of faricimab over time |
| systemic pharmaco- | |
| kinetics of faricimab | |
| Immunogenicity | |
| Objectives | Corresponding Endpoints |
| To evaluate the immune | Presence of ADAs during the study relative |
| response to faricimab | to the presence of ADAs at baseline |
| To evaluate potential | Relationship between ADA status and |
| effects of ADAs | efficacy, safety, or PK endpoints |
| Exploratory | |
| Pharmacokinetic, | |
| Pharmacodynamic, and | |
| Biomarker Objectives | Corresponding Endpoints |
| To identify biomarkers | Concentration of biomarkers of angiogenesis |
| that are predictive of | and inflammation in aqueous humor |
| response to faricimab, | (optional) at baseline and over time and their |
| are associated with | correlation with PK and/or primary and |
| progression to a more | secondary endpoints at baseline and over time |
| severe disease state, are | Relationship between efficacy, safety, PK, |
| associated with | immunogenicity, or other biomarker |
| susceptibility to de- | endpoints and genetic polymorphisms at |
| veloping adverse events, | loci, including, but not limited to, VEGFA |
| can provide evidence of | and ANGPT2 |
| faricimab activity, or can | Relationship between baseline anatomic |
| increase the knowledge | measures and the change in BCVA or other |
| and understanding of | endpoints (e.g., the frequency of study drug |
| disease biology | administration) over time |
| | Relationship between anatomic measures |
| | and visual acuity |
| To evaluate potential | Relationship between selected covariates |
| relationships between | and plasma or aqueous humor (optional) |
| selected covariates and | concentration or PK parameters for faricimab |
| exposure to faricimab | |
| To characterize the | Aqueous humor (optional) and vitreous |
| aqueous humor (optional) | (optional) concentration of faricimab over |
| and vitreous (optional) | time |
| pharmacokinetics | |
| of faricimab | |
| To evaluate the drug | Relationship between pharmacokinetics of |
| concentration (exposure)- | faricimab and concentration of free VEGF-A |
| effect relationship for free | and free Ang-2 in aqueous humor (optional), |
| VEGF-A and free Ang-2 | plasma, and/or vitreous (optional) over time |
| To explore the | Pharmacokinetics of faricimab and the |
| concentration-effect | change in BCVA or other endpoints (e.g., |
| relationship for visual | anatomical markers) over time |
| acuity and other endpoints | |
| (e.g., anatomical markers) | |

^{a}The definition of 1 year is the average of the Week 48, 52, and 56 visits.
^{b} The total retinal area is defined as 7-modified fields or 4-wide fields or ETDRS 7-field mask overlay on ultra-wide field (UWF; Optos ®) images in all study patients and as the entire UWF image, including peripheral areas in a subset of patients with Optos FFA.
^{c} In a subset of patients with OCT-A.
Abbreviations in the Table
ADA = anti-drug antibody; Ang-2 = angiopoietin-2; ANGPT2 = angiopoietin-2 (gene); BCVA = best-corrected visual acuity; CST = central subfield thickness; DR = diabetic retinopathy; DRS = diabetic retinopathy severity; DRSS = Diabetic Retinopathy Severity Scale; ETDRS = Early Treatment Diabetic Retinopathy Study; FFA = fundus fluorescein angiography; IVT = intravitreal; NEI VFQ-25 = National Eye Institute 25-Item Visual Function Questionnaire; OCT-A = optical coherence tomography-angiography; PDR = proliferative diabetic retinopathy; PK = pharmacokinetic; PRP = panretinal photocoagulation; PTI = personalized treatment interval; Q4W = every 4 weeks; Q8W = every 8 weeks; Q12W = every 12 weeks; Q16W = every 16 weeks; SD-OCT = spectral-domain optical coherence tomography; VEGFA = vascular endothelial growth factor-A (gene).

Patients suffering from DME (e.g. center-involving diabetic macular edema (CI-DME)). are treated with the bispecific antibody that binds to human VEGF and human ANG2 comprising the amino acid sequences of SEQ ID NO: 17, of SEQ ID NO: 18, of SEQ ID NO: 19, and of SEQ ID NO: 20 (this antibody VEGFang2-0016 and its production is also described in detail in WO2014/009465 which is incorporated by reference). Designations of this bispecific anti-VEGF/ANG2 antibody herein are RO6867461 or RG7716 or VEGFang2-0016, or faricimab. As active comparator in treatment e.g. aflibercept will be used. Patients include anti-VEGF treatment-naïve patients (have not been previously treated with anti-VEGF treatment with e.g. aflibercept and/or ranibizumab and/or other anti-VEGF treatment)) and also a group of patients which have been previously treated with anti-VEGF treatment. Vials of sterile, colorless to brownish, preservative-free solution of RO6867461(faricimab) for intravitreal (IVT) administration of 6 mg dose are used. RO6867461 (faricimab) will be administered at a concentration of about 120 mg/ml.

Approximately 900 patients will be randomized during the global enrollment phase of the study in a 1:1:1 ratio to one of three treatment arms (see FIG. 2) at approximately 240 investigational sites globally. The study will randomize patients with DME who are naive to anti-VEGF therapy in the study eye and patients who have previously been treated with anti-VEGF therapy in the study eye, provided that the last treatment was at least 3 months prior to the Day 1 visit (the first study treatment). Site investigators will be retina specialists

The study treatment arms will be as follows (see also FIG. 2):

Arm A (administered Q8W): Patients randomized to Arm A will receive 6-mg IVT RO6867461 (faricimab) injections Q4W to Week 20, followed by 6-mg IVT RO6867461 (faricimab) injections Q8W to Week 96, followed by the final study visit at Week 100.

Arm B (personalized treatment interval PTI): Patients randomized to Arm B will receive 6-mg IVT RO6867461 (faricimab) injections Q4W to at least Week 12, followed by PTI dosing (see the PTI dosing criteria below) of 6-mg IVT RO6867461 (faricimab) injections to Week 96, followed by the final study visit at Week 100.

Arm C (comparator arm) (administered Q8W): Patients randomized to Arm C will receive 2-mg IVT aflibercept injections Q4W to Week 16, followed by 2-mg IVT aflibercept injections Q8W to Week 96, followed by the final study visit at Week 100.

Patients in all three treatment arms will complete scheduled study visits Q4W for the entire study duration (100 weeks). A sham procedure will be administered to patients in all three treatment arms at applicable visits to maintain masking among treatment arms (see FIG. 2-Study Treatment Schema).

Only one eye will be assigned as the study eye. If both eyes are considered eligible, the eye with the worse BCVA, as assessed at screening, will be selected as the study eye unless the investigator deems the other eye to be more appropriate for treatment in the study.

There will be a minimum of two investigators per site to fulfill the masking requirements of the study. At least one investigator will be designated as the assessor physician who will be masked to each patient's treatment assignment and who will evaluate ocular assessments. At least one other investigator will be unmasked and will perform study treatments (see Section 4.2.2 for additional masking details).

Treatment Schedule for Patients in the Personalized Treatment Interval (PTI) Arm (Arm B)

The dosing interval decisions in the PTI arm are described in this section. Study drug dosing visits are visits when a patient is assigned to receive faricimab (RO6867461).

Study Drug Dosing Interval Determination

Patients randomized to the PTI arm (Arm B) will be treated with faricimab on a Q4W dosing interval until the patient's Week 12 visit or later CST meets the predefined reference CST threshold (CST <325 μm for Spectralis SD-OCT, or <315 μm for Cirrus SD-OCT or Topcon SD-OCT). The reference CST is used at study drug dosing visits for interval decision-making.

After a patient's initial reference CST is established, their study drug dosing interval will be increased by 4 weeks to an initial Q8W dosing interval. From this point forward, the study drug dosing interval will be extended, reduced, or maintained based on assessments made at study drug dosing visits.

FIG. 3 outlines the algorithm for interval decision-making, which is based on the relative change of the CST and BCVA compared with reference CST and reference BCVA. In FIG. 3 * and ** mean the following:

* Reference central subfield thickness (CST): the CST value when the initial CST threshold criteria are met. Reference CST is adjusted if CST decreases by >10% from the previous reference CST for two consecutive study drug dosing visits and the values obtained are within 30 μm. The CST value obtained at the latter visit will serve as the new reference CST, starting immediately at that visit. ** Reference best-corrected visual acuity (BCVA): the mean of the three best BCVA scores obtained at any prior study drug dosing visit.

All comparisons are made relative to the reference CST* and reference BCVA**. Determination of the drug dosing interval based on CST and BCVA data obtained from the drug dosing visits.

Interval extended by 4 weeks:
If the CST value is increased or decreased by ≤10% without an associated ≤10-letter BCVA decrease

Interval maintained:
If the CST is decreased by >10% or CST value is increased or decreased by ≤10% with an associated ≥10-letter BCVA decrease or
CST value is increased between >10% and ≤20% without an associated ≥5-letter BCVA decrease

Interval reduced by 4 weeks:
If the CST value is increased between >10% and ≤20% with an associated ≥5- to <10-letter BCVA decrease or
CST value is increased by >20% without an associated ≥10-letter BCVA decrease

Interval reduced by 8 weeks:
If the CST value is increased by >10% with an associated ≥10-letter BCVA decrease
* Reference central subfield thickness (CST): the CST value when the initial CST threshold criteria are met. Reference CST is adjusted if CST decreases by >10% from the previous reference CST for two consecutive study drug dosing visits and the values obtained are within 30 μm. The CST value obtained at the latter visit will serve as the new reference CST, starting immediately at that visit.

** Reference best-corrected visual acuity (BCVA): the mean of the three best BCVA scores obtained at any prior study drug dosing visit.

The personalized drug dosing interval can be adjusted by 4-week increments to a maximum of every 16 weeks (Q16W) and a minimum of Q4W. The algorithm for the personalized drug treatment interval decision making is based on the relative change of the CST and absolute change in BCVA compared with the reference CST and BCVA, respectively.

The algorithm may be implemented by a computing system or device. Such a computing system or device may include a web interface, mobile app, software program, or any clinical decision support tool. For example, patient CST and BCVA scores may be uploaded to a web interface of a personalized dosing interval software tool. Using the uploaded CST and BVCA, the tool may automatically compute and output the timing of a next dose. The tool may further provide dosing schedules or notifications, monitor and generate visualizations of dosing interval changes for a given patient, generate visualizations of dosing interval changes for groups of patients, aggregate received CST and BCVA data to determine trends, or a combination thereof.

Dosing schedules or notifications may include displays of calendar dates of scheduled dosing visit(s) and calendar alerts notifying clinicians or patients of upcoming dosing visits. Visualizations of dosing interval changes may include, for instance, displays of the schematics in FIG. 3. In one case, a patient's dosing interval adjustment may be shown in one color, and the patient's immediate prior dosing interval adjustment may be shown in another color. To illustrate, a patient may first have their interval extended by 4 weeks, and then have their personalized treatment interval maintained. The tool may generate a visualization of the patient's personalized interval progression by showing the “interval maintained” area of the schematic in FIG. 3 in green, and the “interval extended by 4 weeks” shown in yellow. Green may reflect the patient's most recent interval computation and yellow may depict results of the patient's immediate prior interval computation. With this visualization, a user of the tool may quickly ascertain that a patient's disease progression is improving, but not so improved that their treatment interval may be extended more.

The tool may further aggregate patient and dosing schedule data and generate visualizations of the aggregated data. Such data analyses may include visualizations of dosing changes for a single patient, similar to the color coding example previously described. Alternately, visualizations may show dosing adjustments across groups of patients. For example, one visualization may show which patients are having interval extensions, and which patients are having interval reductions. This visualization may be organized by various characteristic(s), e.g., patient age, prior treatment, disease state, administered antibody, clinical trial group, etc. The tool may also aggregate and create visualizations from patient CST and BCVA data. The visualizations may show trends in the data to facilitate or generate longitudinal analyses. These visualizations may include alerts, plots, analysis workflow interfaces, or any graphical interface.

The tool may generate dosing schedule outputs or visualizations in response to, or along with ocular assessments and images. In one embodiment, the tool may directly compute patient CST or BVCA. For CST, the tool may receive or directly capture ocular images. The tool may further employ image segmentation, image recognition, or machine learning techniques to compute CST from the ocular images. For BCVA, the tool may administer ocular assessments virtually, prompting and collecting patient user inputs via a user interface or via eye tracking mechanisms. Alternately, the tool may receive, store, and track ocular assessment data. In this way, the tool may track each patient's disease progression and adjust dosing schedules accordingly.

The present embodiments may include a method of providing a personalized dosing schedule according to a personalized treatment interval (PTI) for the treatment of a patient suffering from DME, the method comprising: receiving, at a computing system, patient data comprising a patient's CST and best-corrected visual acuity (BCVA); using the computing system, extending, reducing, or maintaining a dosing interval based on the received patient data compared with respective reference CST and BCVA; and generating a PTI from the dosing interval. The exemplary dosing interval is extended by 4 weeks, if the CST value is increased or decreased by ≤10% without an associated ≥10-letter BCVA decrease. The exemplary dosing interval will be maintained: if the CST is decreased by >10%, the CST value is increased or decreased by ≤10% with an associated ≥10-letter BCVA decrease, or the CST value is increased between >10% and ≤20% without an associated ≥5-letter BCVA decrease. The exemplary dosing interval is reduced by 4 weeks if the CST value is increased between >10% and ≤20% with an associated ≥5 to <10-letter BCVA decrease; or the CST value is increased by >20% without an associated ≥10-letter BCVA decrease. The exemplary dosing interval is reduced by 8 weeks if the CST value is increased by >10% with an associated ≥10-letter BCVA decrease.

Such a method of providing a personalized dosing schedule according to a personalized treatment interval (PTI) for the treatment of a patient suffering from DME, may further comprise receiving, at the computing system, updated patient data; using the computing system, continually updating or maintaining the dosing interval based on the updated patient data; and generating a visualization, user interface, or notification based on the updated or maintained dosing interval.

The present embodiments also include use of a personalized dosing schedule according to a personalized treatment interval (PTI) (for the treatment of DME), wherein a computing system generates the PTI by: receiving patient data comprising a patient's CST and best-corrected visual acuity (BCVA); and extending, reducing, or maintaining a dosing interval based on the received patient data compared with respective reference CST and BCVA. The exemplary dosing interval is extended by 4 weeks, if the CST value is increased or decreased by ≤10% without an associated ≥10-letter BCVA decrease. The exemplary dosing interval will be maintained if the CST is decreased by >10%, or the CST value is increased or decreased by ≤10% with an associated ≥10-letter BCVA decrease, or the CST value is increased between >10% and ≤20% without an associated ≥5-letter BCVA decrease. The exemplary dosing interval is reduced by 4 weeks—if the CST value is increased between >10% and ≤20% with an associated ≥5 to <10-letter BCVA decrease; or the CST value is increased by >20% without an associated ≥10-letter BCVA decrease. The exemplary dosing interval is reduced by 8 weeks if the CST value is increased by >10% with an associated ≥10-letter BCVA decrease. Similar to Arms A and C, patients randomized to the PTI arm (Arm B) will receive a sham procedure at study visits when they are not receiving treatment with faricimab.

Ocular Assessments

Ocular assessments include the following and will be performed for both eyes at specified time points according to the schedule of activities:
Refraction and BCVA assessed on ETDRS chart at a starting distance of 4 meters. BCVA is measured by using the set of three Precision Vision™ or Lighthouse distance acuity charts (modified ETDRS Charts 1, 2, and R). A VA Manual was provided to the investigators. VA examiner and VA examination room certifications were obtained before any VA examinations were performed. The BCVA examiner is masked to study eye and treatment assignment and will only perform the refraction and BCVA assessment (e.g. Visual Acuity Specification Manual). The BCVA examiner is also masked to the BCVA letter scores of a patient's previous visits and only knew the patient's refraction data from previous visits. The BCVA examiner is not allowed to perform any other tasks involving direct patient care.
Pre-treatment IOP (intraocular pressure) measurement of both eyes (perform prior to dilating eyes).
Slitlamp examination (for grading scales for anterior and vitreous cells, see Foster C S, Kothari S, Anesi S D, et al. The Ocular and Uveitis Foundation preferred practice patterns of uveitis management. Sury Opthalmol 61 (2016) 1-17).
Dilated binocular indirect high-magnification ophthalmoscopy.
Finger-counting test followed by hand motion and light perception tests (when necessary) performed within approximately 15 minutes of post-study treatment in the study eye only by the unmasked treatment administrator.
At study treatment visits, post treatment IOP measurement in the study eye only at 30 (±15) minutes by qualified personnel assigned to the unmasked role. If there are no safety concerns after 30 (±15) minutes following the study treatment, the patient will be permitted to leave the clinic. If the TOP value is of concern to the treatment administrator, the patient will remain in the clinic and will be managed in accordance with this physician clinical judgment. The adverse event will be recorded on the Adverse Event electronic Case Report Form (eCRF) as applicable.

The method of TOP measurement used for a patient must remain consistent throughout the study.

Ocular Imaging

The central reading center(s) (CRC(s)) will provide sites with the CRC(s) manual and training materials for specified study ocular images. Before any study images are obtained, site personnel, test images, systems and software (where applicable) will be certified and validated by the CRC(s) as specified in the CRC manual. All ocular images results will be obtained by trained site personnel at the study sites and forwarded to the CRC(s) for independent analysis and/or storage.

After randomization, if a patient misses a study visit when ocular CFP and FFA images are scheduled or the images are not taken at the scheduled visit (e.g., due to broken equipment), they should be obtained at the next scheduled visit the patient attends.

Ocular images include the following:
Mandatory Color Fundus Photography (CFP) (7- or 4-wide fields; perform one of these methods for the patient consistently throughout the trial participation) of both eyes. Stereo color fundus photographs will be obtained from both eyes by trained personnel at the study sites. Fundus photography will be performed at the intervals specified in the schedule of activities.
Optional ultra-wide field (UWF; Optos®) CFP of both eyes (at the sites with UWF CFP capabilities and agreement to take these images in addition to the mandatory CFP images)
Fundus Fluorescein Angiography (FFA) (preferred method is UWF (Optos) FFA if sites have capability; the sites without UWF (Optos) FFA to capture 7 or 4-wide fields using the same method consistently throughout the trial participation) of both eyes (if applicable, performed after blood samples are obtained) will be performed on both eyes at the study sites by trained personnel. UWF (Optos) is the preferred method for fundus fluorescein angiography (FFA) capture. The study sites without Optos equipment and certification must use 7- or 4-wide field FFA capture.
Spectral-Domain Optical Coherence Tomography (SD-OCT) or swept-source OCT (SS-OCT) images of both eyes.
Optional OCT-angiography (OCT-A) of both eyes at sites with OCT-A capabilities and agreement by sites to take these images.

Results

The primary efficacy analyses included all randomized patients, with patients grouped according to the treatment assigned at randomization.

The primary efficacy variable is the BCVA change as described herein. The primary efficacy analysis will be performed using e.g. a Mixed Model for Repeated Measurement (MMRM) model.

Best Corrected Visual Acuity

BCVA is measured as described. Primary Efficacy Outcome Measure is shown in a Figure which displays the primary efficacy endpoint: BCVA change from Baseline over Time for patients. The bispecific anti-VEGF/ANG2 antibody RO6867461 (faricimab) comprising the amino acid sequences of SEQ ID NO: 17, of SEQ ID NO: 18, of SEQ ID NO: 19, and of SEQ ID NO: 20 (administered intravitreally with a 6.0 mg as described in Arm B using the personalized treatment interval), is compared e.g. to Arm A (Faricimab with Q8W dosing) and/or Arm C (aflibercept (Eylea®) Q8W dosing) according to the study scheme described above.

Central Subfield Thickness (CST) Change from Baseline (Study Eye)

A key secondary endpoint is the change from baseline in CST, central subfield thickness. CST (as well as retinal thickness) is measured via Optical coherence tomography (OCT). Results are shown in a Figure in which the change of CST is shown over time for the bispecific anti-VEGF/ANG2 antibody RO6867461 (faricimab) comprising the amino acid sequences of SEQ ID NO: 17, of SEQ ID NO: 18, of SEQ ID NO: 19, and of SEQ ID NO: 20 (administered intravitreally with a 6.0 mg as described in Arm B using the personalized treatment interval), is compared e.g. to Arm A (Faricimab with Q8W dosing) and/or Arm C (aflibercept (Eylea®) Q8W dosing) according to the study scheme described above.

Further outcomes of the ocular assessment and imaging can be displayed accordingly

### Example 3

Efficacy and Durability of Bispecific Anti-VEGF/ANG2 Treatment of Patients Suffering from Macular Edema Secondary to Retinal Vein Occlusion (RVO) (Macular Edema Secondary to Central Retinal Vein Occlusion (CRVO), Secondary to Hemiretinal Vein Occlusion (HRVO) or Secondary to Branch Vein Occlusion(BRVO)) Using a Personalized Treatment Interval

Nonclinical studies have shown that Ang-2 and VEGF act in concert to regulate the vasculature and to increase retinal endothelial cell permeability in vitro. Simultaneous inhibition of Ang-2 and VEGF with the bispecific monoclonal antibody faricimab led to a greater reduction in the leakiness and severity of choroidal neovascularization (CNV) lesions in a laser-induced CNV model in non-human primates compared with the molar equivalent of anti-VEGF (ranibizumab) or anti-Ang-2 alone. Earlier experiments using a mouse model of spontaneous CNV showed that dual inhibition of Ang-2 and VEGF consistently outperformed monotherapeutic inhibition of either target alone in terms of reduction in vascular growth, leakage, edema, leukocyte infiltration, and photoreceptor loss (Regula J T, Lundh von Leithner P, Foxton R, et al. EMBO Mol Med 2016; 8:1265-1288).

In addition, aqueous and vitreous concentrations of both Ang-2 and VEGF were shown to be upregulated in patients with neovascular age-related macular degeneration (nAMD), DR, and RVO (Tong J P, Chan W M, Liu D T, et al. Am J Ophthalmol 2006; 141:456-462; Penn J S, Madan A, Caldwell R B, et al. Prog Retin Eye Res 2008; 27:331-371.; Kinnunen K, Puustjärvi T, Teräsvirta M, et al. Br J Ophthalmol 2009; 93:1109-1115; Tuuminen R, Loukovaara S. Eye (Lond) 2014; 28 :1095-1099; Regula J T, Lundh von Leithner P, Foxton R, et al. EMBO Mol Med 2016; 8:1265-1288; Ng D S, Yip Y W, Bakthavatsalam M, et al. Sci Rep 2017; 7:45081). Therefore, simultaneous neutralization of both targets, Ang-2 and VEGF, may further normalize the pathological ocular vasculature compared with anti-VEGF therapy alone. Data from the completed Phase II studies in DME and nAMD (see below) also support the hypothesis that targeting Ang-2 has the potential to extend the durability of effect beyond anti-VEGF therapy alone in diseases affecting the retinal vasculature.

Faricimab has been studied for the treatment of nAMD and DME in two Phase I studies (BP28936 in nAMD and JP39844 in nAMD and DME) and in three Phase II studies (BP29647 [AVENUE] and CR39521 [STAIRWAY] for nAMD and BP30099 [BOULEVARD] for DME). Four global Phase III studies are ongoing: GR40349 (YOSEMITE) and GR40398 (RHINE) in DME and GR40306 (TENAYA) and GR40844 (LUCERNE) in nAMD.

Based on the mechanism of action of faricimab, data from nonclinical and clinical trials, and the pathophysiology of macular edema due to RVO, it is hypothesized that faricimab may lead to stabilization of the pathological ocular vasculature and to improved visual and anatomical outcomes in RVO compared with anti-VEGF monotherapies.

Macular edema secondary to/due to RVO are among the highest in retinal vascular diseases (Aiello L P, Avery R L, Arrigg P G, et al. N Engl J Med1994; 331:1480-1487; Regula J T, Lundh von Leithner P, Foxton R, et al. EMBO Mol Med 2016; 8:1265-1288). The effect of Ang-2 and VEGF inhibition in the nonclinical models of angiogenesis and inflammation (Regula J T, Lundh von Leithner P, Foxton R, et al. EMBO Mol Med 2016; 8:1265-1288) and the data from Phase I and Phase II faricimab studies in patients with nAMD and DME provide the evidence of efficacy on pathological pathways that are common to all three retinal vascular diseases: nAMD, DME/DR, and macular edema due to RVO (Phase I study BP28936 in nAMD; Phase II studies AVENUE in nAMD, STAIRWAY in nAMD, and BOULEVARD in DME).

Data from the Phase II BOULEVARD study are reported here due to parallels in pathophysiology between DME and macular edema due to RVO. While the trigger for macular edema in diabetic and RVO patients is different, the downstream pathophysiology of hypoxia-driven macular edema with subsequent vision loss is similar and driven by the same proangiogenic, pro-inflammatory, vessel destabilization and vessel permeability factors, including Ang-2, VEGF, and interleukin-6 (IL-6). Results with respect to the potential longer time to retreatment for RO6867461 (faricimab, VA2) are shown in FIG. 6. FIG. 6 shows the time to retreatment in DME patients after dosing has discontinued (after 20 weeks or 6 monthly doses=Time post last intravitreal (IVT) administration) based on disease activity assessed by both: BCVA decreased by ≥5 letters and CST increased by ≥50 μm (=patients with an event). The bispecific anti-VEGF/ANG2 antibody RO6867461 (faricimab) (administered intravitreally with a 6.0 mg or 1.5 mg dose), was compared to ranibizumab (Lucentis®) (administered intravitreally with a 0.3 mg dose).

The BOULEVARD study provided preliminary evidence of a positive benefit/risk profile for the use of 6-mg IVT injections of faricimab for patients with DME and supported further evaluation of faricimab in the Phase III DME studies. The study met its primary efficacy endpoint, demonstrating statistically significant improvement in the mean change from baseline in BCVA at Week 24 in patients naive to anti-VEGF treatment who were treated with 6 mg faricimab compared with 0.3 mg ranibizumab.

The outcomes in the off-treatment study observation period provided evidence of prolonged duration of effect with faricimab compared with anti-VEGF monotherapy.

Assessment of time to disease reactivation up to 16 weeks after the last dose showed an improvement in the duration of the effect of faricimab over ranibizumab, as measured by the time to loss of ≥5 Early Treatment Diabetic Retinopathy Study (ETDRS) letters because of DME and an increase ≥50 μm in central subfield thickness (CST), in the treatment-naive patient population in a dose-dependent manner. This improvement in the duration of effect of faricimab over ranibizumab was also seen in the previously treated group and the overall patient group. Based on the totality of this nonclinical and clinical evidence, treatment with faricimab could lead to improved efficacy over anti-VEGF standard of care in patients with macular edema due to RVO. Additionally, this study will investigate a less frequent treatment administration schedule tailored to individual need (up to every 16 weeks) that could provide BCVA outcomes comparable to those of more frequently administered anti-VEGF monotherapy (e.g., every 4 to 8 weeks). Together, these would represent an important and meaningful advance relative to currently available therapies.

Study design

Phase III, multicenter, randomized, double-masked, active comparator-controlled, parallel-group study evaluating the efficacy, safety, and pharmacokinetics of faricimab (bispecific antibody that binds to human VEGF and human ANG2 comprising the amino acid sequences of SEQ ID NO: 17, of SEQ ID NO: 18, of SEQ ID NO: 19, and of SEQ ID NO: 20 (VEGFang2-0016 WO2014/009465 which is incorporated by reference. Designations of this bispecific anti-VEGF/ANG2 antibody herein are RO6867461 or RG7716 or VEGFang2-0016, or faricimab). administered by IVT injection at 4-week intervals until Week 24, followed by a double-masked period of study without active control to evaluate faricimab administered according to a PTI dosing regimen in patients with macular edema secondary/due to CRVO or HRVO or BRVO was initiated.

Overview of Study Design

This study is comprised of two parts: Part 1 (Day 1 through Week 24) will compare faricimab Q4W versus aflibercept (active comparator) Q4W; Part 2 (Weeks 24-72) will evaluate faricimab administered at masked treatment intervals of Q4W to Q16W based on PTI dosing criteria.

In Part 1 (Q4W Dosing), approximately 680 patients will be randomized during the global enrollment phase of the study in a 1:1 ratio to one of two treatment arms, with treatment defined as follows:
Arm A (n=340): Patients randomly assigned to Arm A will receive faricimab 6 mg IVT Q4W from Day 1 through Week 20 (6 injections).
Arm B (comparator arm, n=340): Patients randomly assigned to Arm B will receive aflibercept 2 mg IVT Q4W from Day 1 through Week 20 (6 injections).

In Part 2 (PTI Regimen), patients in both Arms A and B will receive faricimab 6 mg IVT according to a PTI dosing regimen from Week 24 through Week 68

All patients will complete scheduled study visits Q4W for the entire study duration (72 weeks). To preserve the masking of faricimab treatment intervals for Week 24 through Week 68, a sham procedure will be administered during study visits at which (according to the PTI dosing regimen) no faricimab treatment is administered.

FIG. 7 presents an overview of the study design.

Only one eye will be assigned as the study eye. If both eyes are considered eligible, the eye with the worse BCVA, as assessed at screening, will be selected as the study eye, unless the investigator deems the other eye to be more appropriate for treatment in the study. There will be a minimum of two investigators per site to fulfill the masking requirements of the study. At least one investigator will be designated as the assessor physician who will be masked to each patient's treatment assignment and who will evaluate ocular assessments. At least one other investigator will be unmasked and will perform study treatments.

The study will consist of a screening period of up to 28 days (Days −28 to −1) and an approximately 68-week treatment period, followed by the final study visit at Week 72.

Objectives And Endpoints

This study will evaluate the efficacy, safety, and pharmacokinetics of faricimab compared with aflibercept in patients with macular edema secondary to (due to) CRVO or HRVO or BRVO up to the primary endpoint at Week 24. Efficacy, safety, and pharmacokinetics of faricimab administered according to the PTI dosing regimen (i.e., from Q4W to Q16W) will be assessed during the study period from Week 24 to Week 72. Specific objectives and corresponding endpoints for the study are outlined below. In this protocol, “study drug” refers to faricimab or aflibercept and “study treatment” refers to faricimab, aflibercept, or the sham procedure.

Efficacy Objectives

For efficacy endpoint evaluation, BCVA will be assessed on the ETDRS visual acuity chart at a starting test distance of 4 meters.

Primary Efficacy Objective

The primary efficacy objective for this study is to evaluate the efficacy of faricimab 6 mg IVT Q4W compared with aflibercept 2 mg IVT Q4W on the basis of the following endpoint:

Change from baseline in BCVA at Week 24

Secondary Efficacy Objectives

The secondary efficacy objective for Part 1 of this study (i.e., through Week 24) is to evaluate the efficacy of faricimab compared with aflibercept on the basis of the following endpoints:

Change from baseline in BCVA at specified time points through Week 24

Proportion of patients with an increase from baseline of ≥15 letters in BCVA at Week 24
Proportion of patients with an increase from baseline of ≥15, ≥10, ≥5, or >0 letters in BCVA at specified time points through Week 24
Proportion of patients avoiding a loss of ≥15, ≥10, ≥5, or >0 letters in BCVA from baseline at specified time points through Week 24
Proportion of patients achieving ≥84 letters (20/20 Snellen equivalent) in BCVA at specified time points through Week 24
Proportion of patients with BCVA Snellen equivalent of 20/40 or better at specified time points through Week 24
Proportion of patients with BCVA Snellen equivalent of 20/200 or worse at specified time points through Week 24
Change from baseline in CST at specified time points through Week 24
Change from baseline in National Eye Institute 25-Item Visual Functioning Questionnaire (NEI VFQ-25) composite score at specified time points through Week 24

The secondary efficacy objective for Part 2 of this study (i.e., Week 24 through Week 72) is to evaluate and the efficacy of faricimab administered according to the PTI dosing regimen on the basis of the following endpoints:
Change from baseline in BCVA at specified time points from Week 24 through Week 72
Proportion of patients with an increase from baseline of ≥15 letters in BCVA at Week 24
Proportion of patients with an increase from baseline of ≥15, ≥10, ≥5, or >0 letters in BCVA at specified time points from Week 24 through Week 72
Proportion of patients avoiding a loss of ≥15, ≥10, ≥5, or >0 letters in BCVA from baseline at specified time points from Week 24 through Week 72
Proportion of patients achieving ≥84 letters (20/20 Snellen equivalent) in BCVA at specified time points from Week 24 through Week 72
Proportion of patients with BCVA Snellen equivalent of 20/40 or better at specified time points from Week 24 through Week 72
Proportion of patients with BCVA Snellen equivalent of 20/200 or worse at specified timepoints from Week 24 through Week 72
Change from Week 24 in BCVA at specified timepoints through Week 72
Proportion of patients avoiding a loss of ≥15, ≥10, ≥5, or >0 letters in BCVA from Week 24 through Week 72
Proportion of patients on a Q4W, every 8 weeks (Q8W), every 12 weeks (Q12W), or Q16W treatment interval at Week 72
Number of study drug injections received from Week 24 through Week 72
Change from baseline in CST at specified timepoints from Week 24 through Week 72
Change from baseline in NEI VFQ-25 composite score at specified timepoints from Week 24 through Week 72

Exploratory Efficacy Objective

The exploratory efficacy objective for this study is to evaluate the efficacy of faricimab on the basis of the following endpoints:
Proportion of patients with absence of retinal ischemia on fundus fluorescein angiography (FFA) and on optical coherence tomography angiography (OCT-A) (optional) over time (at specified timepoints)
Change from baseline in area of retinal ischemia on FFA and on OCT-A (optional) over time
Proportion of patients with vascular leakage on FFA and on OCT-A (optional) over time
Change from baseline in area of vascular leakage on FFA, and on OCT-A (optional) over time
Change from baseline in foveal avascular zone and other exploratory outputs defined in SAP (Statistical Analysis Plan) on OCT-A (optional) over time
Proportion of patients with absence of retinal neovascularization (per investigator assessment) over time
Proportion of patients with absence of vitreal, preretinal, or subretinal hemorrhage over time (per investigator assessment)
Proportion of patients with absence of anterior segment (iris and anterior chamber angle) neovascularization over time
Proportion of patients requiring panretinal photocoagulation at any time during study
Proportion of patients with absence of macular edema, defined as CST of ≤325 μm for Spectralis SD-OCT, or ≤315 μm for Cirrus SD-OCT or Topcon SD-OCT, over time
Proportion of patients with absence of intraretinal fluid over time
Proportion of patients with absence of subretinal fluid over time
Proportion of patients with absence of both intraretinal fluid and subretinal fluid over time
Proportion of patients with absence of intraretinal cysts over time
Change from baseline in NEI VFQ-25 near activities-subscale score and distance activities-subscale scores over time

Treatment Schedule for Part 1 (Q4W Dosing)

In Part 1 of the study, patients will receive treatment as follows:
Patients randomly assigned to Arm A will receive faricimab Q4W from Day 1 through Week 20
Patients randomly assigned to Arm B will receive aflibercept Q4W from Day 1 through Week 20

Treatment Schedule for Part 2 (Personalized Treatment interval (PTI) Regimen)

In Part 2 of the study, all patients will visit the clinic Q4W from Week 24 through Week 68 and receive either sham treatment or faricimab 6 mg IVT, depending on their PTI dosing regimen.

Faricimab PTI decisions will be automatically calculated based on the PTI criteria described in this section.

Study drug dosing interval decisions in the PTI arm are based on the algorithm described in this section. Faricimab dosing visits are defined as those visits when the patient receives faricimab 6 mg IVT.

Starting at Week 24, patients will receive faricimab at a frequency of Q4W until CST meets the predefined reference CST threshold (<325 mm for Spectralis SD-OCT or <315 mm for Cirrus SD-OCT and Topcon SD-OCT), as determined by the CRC. The reference CST (as defined in FIG. 8 description and below) is used at faricimab dosing visits to determine the faricimab dosing interval. After a patient's initial reference CST is established, the patient is eligible to have the faricimab dosing interval increased in 4-week increments if the CST value is stable (i.e., has not increased or decreased by >10%) with no associated loss of vision of ≥10 letters with respect to reference BCVA (as defined in FIG. 8 description and below).

Reference CST and reference BCVA (in FIG. 8 and figure description see letters ^{a} and ^{b}) mean the following:

a Reference central subfield thickness (CST): the CST value when the initial CST threshold criteria are met. Reference CST is adjusted if CST decreases by >10% from the previous reference CST for two consecutive study drug dosing visits and the values obtained are within 30 μm. The CST value obtained at the latter visit will serve as the new reference CST, starting immediately at that visit.
b Reference best-corrected visual acuity (BCVA): the mean of the three best BCVA scores obtained at any prior study drug dosing visit.

The maximum and minimum treatment intervals that may be assigned will be Q16W and Q4W, respectively. Patients whose dosing interval had been previously extended and who experience disease worsening that triggers interval reduction will not be allowed to extend the interval again, with the exception of patients whose dosing intervals were reduced to Q4W; their interval may be extended again but only to an interval that is 4 weeks less than their original maximum extension. For example, if a patient's interval is reduced from Q12W to Q8W, this patient's interval will not be extended beyond Q8W for the remainder of the treatment period. If a patient's interval is reduced from Q16W to Q4W, this patient's interval can be extended up to Q12W, but cannot be extended back to Q16W.

Faricimab (RO6867461/RG7716/VEGFang2-0016) Interval Determination

The algorithm used for interval decision-making, which is based on the relative change of the CST and BCVA at faricimab dosing visits compared with the reference CST and reference BCVA, is outlined below and in FIG. 8. The faricimab dosing interval will be extended, maintained, or reduced as follows.

Interval extended by 4 weeks

If the CST value is increased or decreased by ≤10% without an associated ≥10-letter BCVA decrease

Interval maintained if any of the following criteria are met:

If the CST value is decreased by >10%

If the CST value is decreased ≤10% with an associated ≥10-letter BCVA decrease

If the CST value is increased between >10% and ≤20% without an associated ≥5-letter BCVA decrease

Interval reduced by 4 weeks if any of the following criteria are met:

If the CST value is increased between >10% and ≤20% with an associated ≥5-to <10-letter BCVA decrease

If the CST value is increased by >20% without an associated ≥10-letter BCVA decrease

If the CST value is increased by <10% with an associated BCVA decrease of ≥10-letters

Interval reduced to Q4W

If the CST value is increased by >10% with an associated ≥10-letter BCVA Decrease

As outlined above the algorithm for the personalized drug treatment interval decision making is based on the relative change of the CST and absolute change in BCVA compared with the reference CST and BCVA, respectively.

The algorithm may be implemented by a computing system or device. Such a computing system or device may include a web interface, mobile app, software program, or any clinical decision support tool. For example, patient CST and BCVA scores may be uploaded to a web interface of a personalized dosing interval software tool. Using the uploaded CST and BVCA, the tool may automatically compute and output the timing of a next dose. The tool may further provide dosing schedules or notifications, monitor and generate visualizations of dosing interval changes for a given patient, generate visualizations of dosing interval changes for groups of patients, aggregate received CST and BCVA data to determine trends, or a combination thereof.

Dosing schedules or notifications may include displays of calendar dates of scheduled dosing visit(s) and calendar alerts notifying clinicians or patients of upcoming dosing visits. Visualizations of dosing interval changes may include, for instance, displays of the schematics in FIG. 8. In one case, a patient's dosing interval adjustment may be shown in one color, and the patient's immediate prior dosing interval adjustment may be shown in another color. To illustrate, a patient may first have their interval extended by 4 weeks, and then have their personalized treatment interval maintained. The tool may generate a visualization of the patient's personalized interval progression by showing the “interval maintained” area of the schematic in FIG. 8 in green, and the “interval extended by 4 weeks” shown in yellow. Green may reflect the patient's most recent interval computation and yellow may depict results of the patient's immediate prior interval computation. With this visualization, a user of the tool may quickly ascertain that a patient's disease progression is improving, but not so improved that their treatment interval may be extended more.

The tool may further aggregate patient and dosing schedule data and generate visualizations of the aggregated data. Such data analyses may include visualizations of dosing changes for a single patient, similar to the color coding example previously described. Alternately, visualizations may show dosing adjustments across groups of patients. For example, one visualization may show which patients are having interval extensions, and which patients are having interval reductions. This visualization may be organized by various characteristic(s), e.g., patient age, prior treatment, disease state, administered antibody, clinical trial group, etc. The tool may also aggregate and create visualizations from patient CST and BCVA data. The visualizations may show trends in the data to facilitate or generate longitudinal analyses. These visualizations may include alerts, plots, analysis workflow interfaces, or any graphical interface.

The tool may generate dosing schedule outputs or visualizations in response to, or along with ocular assessments and images. In one embodiment, the tool may directly compute patient CST or BVCA. For CST, the tool may receive or directly capture ocular images. The tool may further employ image segmentation, image recognition, or machine learning techniques to compute CST from the ocular images. For BCVA, the tool may administer ocular assessments virtually, prompting and collecting patient user inputs via a user interface or via eye tracking mechanisms. Alternately, the tool may receive, store, and track ocular assessment data. In this way, the tool may track each patient's disease progression and adjust dosing schedules accordingly.

The present embodiments may include a method of providing a personalized dosing schedule according to a personalized treatment interval (PTI) for the treatment of a patient suffering from an ocular vascular disease selected from macular edema secondary to central retinal vein occlusion, secondary to hemiretinal vein occlusion or secondary to branch vein occlusion, the method comprising: receiving, at a computing system, patient data comprising a patient's CST and best-corrected visual acuity (BCVA); using the computing system, extending, reducing, or maintaining a dosing interval based on the received patient data compared with respective reference CST and BCVA; and generating a PTI from the dosing interval. The exemplary dosing interval is extended by 4 weeks if the CST value is increased or decreased by ≤10% without an associated ≥10-letter BCVA decrease. The exemplary dosing interval is maintained if any of the following criteria are met: if the CST value is decreased by >10%; or if the CST value is decreased ≤10% with an associated ≥10-letter BCVA decrease; or if the CST value is increased between >10% and ≤20% without an associated ≥5-letter BCVA decrease. The exemplary dosing interval is reduced by 4 weeks if any of the following criteria are met: if the CST value is increased between >10% and ≤20% with an associated ≥5-to <10-letter BCVA decrease, or if the CST value is increased by >20% without an associated ≥10-letter BCVA decrease, or if the CST value is increased by ≤10% with an associated BCVA decrease of ≥10-letters. The exemplary dosing interval is reduced to Q4W if the CST value is increased by >10% with an associated ≥10-letter BCVA decrease.

Such a method of providing a personalized dosing schedule according to a personalized treatment interval (PTI) for the treatment of a patient suffering from an ocular vascular disease selected from macular edema secondary to central retinal vein occlusion, secondary to hemiretinal vein occlusion or secondary to branch vein occlusion may further comprise receiving, at the computing system, updated patient data; using the computing system, continually updating or maintaining the dosing interval based on the updated patient data; and generating a visualization, user interface, or notification based on the updated or maintained dosing interval.

The present embodiments also include use of a personalized dosing schedule according to a personalized treatment interval (PTI) (for the treatment of macular edema secondary to central retinal vein occlusion, secondary to hemiretinal vein occlusion or secondary to branch vein occlusion), wherein a computing system generates the PTI by receiving patient data comprising a patient's CST and best-corrected visual acuity (BCVA) and extending, reducing, or maintaining a dosing interval based on the received patient data compared with respective reference CST and BCVA. The exemplary dosing interval is extended by 4 weeks if the CST value is increased or decreased by ≤10% without an associated ≥10-letter BCVA decrease. The exemplary dosing interval is maintained if any of the following criteria are met: if the CST value is decreased by >10%; or if the CST value is decreased ≤10% with an associated ≥10-letter BCVA decrease; or if the CST value is increased between >10% and ≤20% without an associated ≥5-letter BCVA decrease. The exemplary dosing interval is reduced by 4 weeks if any of the following criteria are met: if the CST value is increased between >10% and ≤20% with an associated ≥5-to <10-letter BCVA decrease, or if the CST value is increased by >20% without an associated ≥10-letter BCVA decrease, or if the CST value is increased by ≤10% with an associated BCVA decrease of ≥10-letters. The exemplary dosing interval is reduced to Q4W if the CST value is increased by >10% with an associated ≥10-letter BCVA decrease.

Ocular Assessments

Ocular assessments will be performed for both eyes, unless otherwise indicated, at specified timepoints according to the schedule of activities. Assessments include:
Refraction and BCVA assessed on ETDRS visual acuity chart at a starting test distance of 4 meters (perform prior to dilating eyes)
Predose TOP measurement of both eyes (perform prior to dilating eyes)
Slitlamp examination (for grading scales for anterior and vitreous cells)
Dilated binocular indirect high-magnification ophthalmoscopy
Finger-counting test followed by hand-motion and light-perception tests (when necessary) performed within approximately 15 minutes of study treatment in the study eye only
Postdose TOP (intraocular pressure) measurement only in the study eye taken 30 (±15) minutes after study treatment administration

If there are no safety concerns after 30 (±15) minutes following study treatment administration, the patient will be permitted to leave the clinic. If the IOP value is of concern to the treatment administrator/unmasked investigator, the patient will remain in the clinic and will be managed in accordance with the treatment administrator/unmasked investigator's clinical judgment. The adverse event will be recorded on the Adverse Event electronic Case Report Form (eCRF) as applicable.
The method of IOP measurement used for a patient must remain consistent throughout the study Ocular Imaging

After randomization, if a patient misses a study visit when Color Fundus Photography (CFP) or Fundus Fluorescein Angiography (FFA) ocular images are scheduled or the images are not taken at the scheduled visit (e.g., due to broken equipment), they should be obtained at the next scheduled visit the patient attends.

Ocular images include the following:
FFA of study eye
CFP of study eye
Spectral-Domain Optical Coherence Tomography (SD-OCT) or swept-source OCT (SS-OCT) images of study eye
Optional OCT-A of study eye at sites with OCT-A capabilities (provided sites approve optional sampling)

For patients diagnosed at screening with bilateral RVO, CFP and OCT images will also be captured of the fellow eye and stored at the CRC.

Results

The primary efficacy analyses included all randomized patients, with patients grouped according to the treatment assigned at randomization.

The primary efficacy variable is the BCVA change. The primary efficacy analysis will be performed using e.g. a Mixed Model for Repeated Measurement (MMRM) model.

Best Corrected Visual Acuity

BCVA is measured as described. Primary Efficacy Outcome Measure is shown in a Figure which displays the primary efficacy endpoint: BCVA change from Baseline over Time for patients. The bispecific anti-VEGF/ANG2 antibody RO6867461 (faricimab) comprising the amino acid sequences of SEQ ID NO: 17, of SEQ ID NO: 18, of SEQ ID NO: 19, and of SEQ ID NO: 20 (administered intravitreally with a 6.0 mg as described in Arm A using the personalized treatment interval), is e.g.

compared to Arm B (aflibercept (Eylea®) in Part 1 of the study) according to the study scheme described above.

Central Subfield Thickness (CST) Change from Baseline (Study Eye)

A key secondary endpoint is the change from baseline in CST, central subfield thickness. CST (as well as retinal thickness) is measured via Optical coherence tomography (OCT). Results are shown in a Figure in which the change of CST is shown over time for the bispecific anti-VEGF/ANG2 antibody RO6867461 (faricimab) comprising the amino acid sequences of SEQ ID NO: 17, of SEQ ID NO: 18, of SEQ ID NO: 19, and of SEQ ID NO: 20 (administered intravitreally with a 6.0 mg as described in Arm A using the personalized treatment interval), is e.g.

compared to Arm B (aflibercept (Eylea®) in Part 1 of the study) according to the study scheme described above.

Further outcomes of the ocular assessment and imaging can be displayed accordingly.

### Example 4

Binding to of the anti-VEGF/ANG2 Antibody to VEGF, Ang2, FcgammaR and FcRn

VEGF Isoforms Kinetic Affinity Including Assessment of Species-Crossreactivity

Around 12000 resonance units (RU) of the capturing system (10 μg/ml goat anti human F(ab)′₂; Order Code: 28958325; GE Healthcare Bio-Sciences AB, Sweden) were coupled on a CMS chip (GE Healthcare BR-1005-30) at pH 5.0 by using an amine coupling kit supplied by the GE Healthcare. The sample and system buffer was PBS-T (10 mM phosphate buffered saline including 0.05% Tween® 20) pH 7.4. The flow cell was set to 25° C.—and the sample block set to 12° C.—and primed with running buffer twice. The bispecific antibody was captured by injecting a 50 nM solution for 30 sec at a flow of 5 μl/min. Association was measured by injection of human hVEGF121, mouse mVEGF120 or rat rVEGF164 in various concentrations in solution for 300 sec at a flow of 30 μl/min starting with 300 nM in 1:3 dilutions. The dissociation phase was monitored for up to 1200 sec and triggered by switching from the sample solution to running buffer. The surface was regenerated by 60 sec washing with a Glycine pH 2.1 solution at a flow rate of 30 μl/min. Bulk refractive index differences were corrected by subtracting the response obtained from a goat anti human F(ab′)₂ surface. Blank injections are also subtracted (=double referencing). For calculation of apparent K_{D} and other kinetic parameters the Langmuir 1:1 model was used. Results are shown in Table 5.

Ang2 Solution Affinity Including Assessment of Species-Crossreactivity

Solution affinity measures the affinity of an interaction by determining the concentration of free interaction partners in an equilibrium mixture. The solution affinity assay involves the mixing of an <VEGF-ANG-2> bispecific antibody, kept at a constant concentration, with a ligand (=Ang2) at varying concentrations. Maximum possible resonance units (e.g. 17000 resonance units (RU)) of an antibody was immobilized on the CM5 chip (GE Healthcare BR-1005-30) surface at pH 5.0 using an amine coupling kit supplied by the GE Healthcare. The sample and system buffer was HBS-P pH 7.4. Flow cell was set to 25° C. and sample block to 12° C. and primed with running buffer twice. To generate a calibration curve increasing concentrations of Ang2 were injected into a BIAcore™ flowcell containing the immobilized VEGF-ANG-2>bispecific antibody. The amount of bound Ang2 was determined as resonance units (RU) and plotted against the concentration. Solutions of each ligand (11 concentrations from 0 to 200 nM for the VEGF-ANG-2>bispecific antibody) were incubated with 10 nM Ang2 and allowed to reach equilibrium at room temperature. Free Ang2 concentrations were determined from calibration curve generated before and after measuring the response of solutions with known amounts of Ang2. A 4-parameter fit was set with XLfit4 (IDBS Software) using Model 201 using free Ang2 concentration as y-axis and used concentration of antibody for inhibition as x-axis. The affinity was calculated by determining the inflection point of this curve. The surface was regenerated by one time 30 sec washing with a 0.85% H₃PO₄ solution at a flow rate of 30 μl/min. Bulk refractive index differences were corrected by subtracting the response obtained from a blank-coupled surface. Results are shown in Tables below.

FeRn Steady State Affinity

For FcRn measurement a steady state affinity was used to compare bispecific antibodies against each other. Human FcRn was diluted into coupling buffer (10 μg/ml, Na-Acetate pH5.0) and immobilized on a C1-Chip (GE Healthcare BR-1005-35) by targeted immobilization procedure using a BIAcore™ wizard to a final response of 200 RU. Flow cell was set to 25° C. and sample block to 12° C. and primed with running buffer twice. The sample and system buffer was PBS-T (10 mM phosphate buffered saline including 0.05% Tween® 20) pH 6.0. To assess different IgG concentrations for each antibody, a concentration of 62.5 nM, 125 nM and 250 nM, 500 nM was prepared. Flow rate was set to 30 μl/min and the different samples were injected consecutively onto the chip surface choosing 180 sec association time. The surface was regenerated by injected PBS-T pH 8 for 60 sec at a flow rate of 30 μl/min. Bulk refractive index differences were corrected by subtracting the response obtained from a blank surface. Buffer injections are also subtracted (=double referencing). For calculation of steady state affinity, the method from the Bia-Evaluation software was used. Briefly, the RU values (RU max) were plotted against the analysed concentrations, yielding a dose-response curve. Based on a 2-parametric fit, the upper asymptote is calculated, allowing the determination of the half-maximal RU value and hence the affinity. Results are shown in the Tables below. Analogously the affinity to cyno, mouse and rabbit FcRn can be determined.

FcgammaRIIIa Measurement

For FcgammaRIIIa measurement a direct binding assay was used. Around 3000 resonance units (RU) of the capturing system (1 μg/ml Penta-His; Qiagen) were coupled on a CMS chip (GE Healthcare BR-1005-30) at pH 5.0 by using an amine coupling kit supplied by the GE Healthcare. The sample and system buffer was HBS-P+pH 7.4. The flow cell was set to 25° C.—and sample block to 12° C.—and primed with running buffer twice. The FcgammaRIIIa -His-receptor was captured by injecting a 100 nM solution for 60 sec at a flow of 5 μl/min. Binding was measured by injection of 100 nM of bispecific antibody or monospecific control antibodies (anti-Dig for IgG1 subclass and an IgG4 subclass antibody) for 180 sec at a flow of 30 μl/. The surface was regenerated by 120 sec washing with Glycine pH 2.5 solution at a flow rate of 30 μl/min. Because FcgammaRIIIa binding differs from the Langmuir 1:1 model, only binding/no binding was determined with this assay. In a similar manner FcgammaRIa, and FcgammaRIIa binding can be determined. Results are shown in the tables below, where it follows that by introduction of the mutations P329G LALA no more binding to FcgammaRIIIa could be detected.

Assessment of Independent VEGF- and Ang2-Binding to the <VEGF-ANG-2> Bispecific Antibodies

Around 3500 resonance units (RU) of the capturing system (10 μg/ml goat anti human IgG; GE Healthcare Bio-Sciences AB, Sweden) were coupled on a CM4 chip (GE Healthcare BR-1005-34) at pH 5.0 by using an amine coupling kit supplied by the GE Healthcare. The sample and system buffer was PBS-T (10 mM phosphate buffered saline including 0.05% Tween® 20) pH 7.4. The temperature of the flow cell was set to 25° C. and of the sample block to 12° C. Before capturing, the flow cell was primed with running buffer twice.

The bispecific antibody was captured by injecting a 10 nM solution for 60 sec at a flow of 5 μl/min. Independent binding of each ligand to the bispecific antibody was analysed by determining the active binding capacity for each ligand, either added sequentially or simultaneously (flow of 30 μl/min):
1. Injection of human VEGF with a concentration of 200 nM for 180 sec (identifies the single binding of the antigen).
2. Injection of human Ang2 with a concentration of 100 nM for 180 sec (identifies single binding of the antigen).
3. Injection of human VEGF with a concentration of 200 nM for 180 sec followed by an additional injection of human Ang2 with a concentration of 100 nM for 180 sec (identifies binding of Ang2 in the presence of VEGF).
4. Injection of human Ang2 with a concentration of 100 nM for 180 sec followed by an additional injection of human VEGF with a concentration of 200 nM (identifies binding of VEGF in the presence of Ang2).
5. Co-Injection of human VEGF with a concentration of 200 nM and of human Ang2 with a concentration of 100 nM for 180 sec (identifies the binding of VEGF and of Ang2 at the same time).

The surface was regenerated by 60 sec washing with a 3mM MgCl2 solution at a flow rate of 30 μl/min. Bulk refractive index differences were corrected by subtracting the response obtained from a goat anti human IgG surface.

The bispecific antibody is able to bind both antigens mutual independently if the resulting final signal of the approaches 3, 4 & 5 equals or is similar to the sum of the individual final signals of the approaches 1 and 2. Results are shown in the Table below, where VEGFang2-0016 (=RO6867461), is shown to be able to bind mutual independently to VEGF and ANG2

Assessment of Simultaneous VEGF- and Ang2-Binding to the <VEGF-ANG-2> Bispecific Antibodies

First, around 1600 resonance units (RU) of VEGF (20m/m1) were coupled on a CM4 chip (GE Healthcare BR-1005-34) at pH 5.0 by using an amine coupling kit supplied by the GE Healthcare. The sample and system buffer was PBS-T (10 mM phosphate buffered saline including 0.05% Tween® 20) pH 7.4. Flow cell was set to 25° C. and sample block to 12° C. and primed with running buffer twice. Second, 50nM solution of the bispecific antibody was injected for 180 sec at a flow of 30 μl/min. Third, hAng-2 was injected for 180 sec at a flow of 30 μl/min. The binding response of hAng-2 depends from the amount of the bispecific antibody bound to VEGF and shows simultaneous binding. The surface was regenerated by 60 sec washing with a 0.85% H3PO4 solution at a flow rate of 30 μl/min. Simultaneous binding is shown by an additional specific binding signal of hAng2 to the previous VEGF bound <VEGF-ANG-2> bispecific antibodies.

TABLE
Results: Kinetic affinities to VEGF isoforms from different species

| | VEGFang2-0016-apparent affinity |
|---|---|
| Human VEGF 121 | ≤1 pM (out of Biacore specification) |
| mouseVEGF 120 | no binding |
| Rat VEGF 164 | 14 nM |

TABLE
Results: Solution affinities to Ang2

| | VEGFang2-0016 KD [nM] |
|---|---|
| humanAng2 | 20 |
| cynoAng2 | 13 |
| mouseAng2 | 13 |
| rabbitAng2 | 11 |

TABLE
Results: Affinity to FcRn of <VEGF-ANG-2> bispecific antibodies

| | VEGFang2-0016 [affinity] |
|---|---|
| Human FcRn | no binding |
| Cyno FcRn | no binding |
| Mouse FcRn | no binding |

TABLE
Results Binding to FcgammaIIIa

| | VEGFang2-0016 |
|---|---|
| FcγRIIIa | No binding |

TABLE
Results: Independent binding of VEGF- and Ang2 to
<VEGF-ANG-2> bispecific antibodies

| | | | 3) first | 4) first | 5) |
|---|---|---|---|---|---|
| | | | VEGF | Ang2 | Coinjection |
| | | | then | then | Ang2 + |
| | 1) Ang2 | 2) VEGF | Ang2 | VEGF | VEGF |
| | [RUmax] | [RUmax] | [RUmax] | [RUmax] | [RUmax] |
| VEGFang2- | 174 | 50 | 211 | 211 | 211 |
| 0016 | | | | | |

1. A bispecific antibody which binds to human vascular endothelial growth factor (VEGF) and to human angiopoietin-2 (ANG-2), for use in the treatment of an ocular vascular diseases selected from neovascular AMD (nAMD) and diabetic macular edema (DME) or of patients suffering from an ocular vascular diseases selected from neovascular AMD (nAMD) and diabetic macular edema (DME), wherein the treatment includes a personalized treatment interval (PTI).

2. The bispecific antibody (for use) according to claim 1, for use in the treatment of neovascular age-related macular degeneration (nAMD) or of patients suffering from nAMD.

3. The bispecific antibody (for use) according to claim 2, wherein the treatment includes a personalized treatment interval, wherein
a) patients are treated first 4 times with the bispecific VEGF/ANG2 antibody at an every 4 weeks (Q4W) dosing interval
b) at Weeks 20 and 24 the disease activity is assessed wherein the disease activity is determined if one of the following criteria are met:
i) increase of >50 μm in central subfield thickness (CST) compared with the average CST value over the previous two scheduled visits which Weeks 12 and 16 for the Week 20 assessment and Weeks 16 and 20 for the Week 24 assessment, or
ii) increase ≥75 μm in CST compared with the lowest CST value recorded at either of the previous two scheduled visits;
iii) decrease ≥5 letters in best-corrected visual acuity (BCVA) compared with average BCVA value over the previous two scheduled visits,
iv) decrease 10 letters in BCVA compared with the highest BCVA value recorded at either of the previous two scheduled visits, or
v) presence of new macular hemorrhage, owing to nAMD activity
c) then patients
i) patients who meet the disease activity criteria at Week20 will be treated at an every 8 weeks (Q8W) dosing interval from week 20 onward (with the first Q8W dosing at Week20);
ii) patients who meet the disease activity criteria at Week24 will be treated at an every 12 weeks (Q12W) dosing interval from week 24 onward (with the first Q12W dosing at Week24); and
iii) patients who do not meet disease activity criteria at Week20 and Week24 will be treated at an every 16 weeks (Q16W) dosing interval from week 28 onward (with the first Q16W dosing at Week28)

4. The bispecific antibody for use according to claim 3, wherein the personalized treatment interval will be extended, reduced, or maintained after week 60 wherein the
a) interval is extended by 4 weeks (to a maximum of Q16W) if all of the following criteria are met:
i) stable CST compared with the average of the last 2 study drug dosing visits where stability is defined as a change of CST of less than 30 μm and no increase ≥50 μm in CST compared with the lowest on-study drug dosing visit measurement,
ii) no decrease ≥5 letters in BCVA compared with the average from the last two study drug dosing visits, and no decrease ≥10 letters in BCVA compared with the highest on-study drug dosing visit measurement,
iii) no new macular hemorrhage
b) interval
is reduced (to a minimum Q8W) by 4 weeks if one of the following criteria is met,
or
is reduced to an 8-week interval if two or more of the following criteria are met or one criterion includes new macular hemorrhage:
i) increase of ≥50 μm in CST compared with the average from the last two dosing visits or of ≥75 μm compared with the lowest dosing visit measurement;
ii) decrease of ≥5 letters in BCVA compared with average of last two dosing visits or decrease ≥10 letters in BCVA compared with the highest dosing visit measurement;
iii) new macular hemorrhage.

5. The bispecific antibody for use according to claim 1, for use in the treatment of diabetic macular edema (DME) or of patients suffering from DME.

6. The bispecific antibody for use according to claim 5 wherein the treatment includes a personalized treatment interval (PTI), wherein
a) patients are treated first with the bispecific VEGF/ANG2 antibody at an every 4 weeks (Q4W) dosing interval until the central subfield thickness (CST) meets a predefined reference CST threshold as measured at week 12 or later;
b) then the dosing interval is increased by 4 weeks to an initial Q8W dosing interval;
c) from this point forward, the dosing interval is extended, reduced, or maintained based on assessments made at the dosing visits. which are based on the relative change of the CST and best-corrected visual acuity (BCVA) compared with the respective reference CST and BCVA;
wherein the
i) interval is extended by 4 weeks,
if the CST value is increased or decreased by ≤10% without an associated ≥10-letter BCVA decrease;
ii) interval will be maintained:
if the CST is decreased by >10%, or
the CST value is increased or decreased by ≤10% with an associated ≥10-letter BCVA decrease, or
the CST value is increased between >10% and ≤20% without an associated ≥5-letter BCVA decrease;
iii) interval is reduced by 4 weeks
if the CST value is increased between >10% and ≤20% with an associated ≥5 to <10-letter BCVA decrease; or
the CST value is increased by >20% without an associated >10-letter BCVA decrease;
iv) interval is reduced by 8 weeks if the CST value is increased by >10% with an associated ≥10-letter BCVA decrease;
wherein the respective reference central subfield thickness (CST) is the CST value when the initial CST threshold criteria are met and the reference CST is adjusted if CST decreases by >10% from the previous reference CST for two consecutive dosing visits and the values obtained are within 30 μm so that the CST value obtained at the latter visit will serve as the new reference CST; and
wherein the reference best-corrected visual acuity (BCVA) is the mean of the three best BCVA scores obtained at any prior dosing visit.

7. The bispecific antibody for use according to the claim 6, wherein the dosing interval can by adjusted by 4-week increments to a maximum of every 16 weeks (Q16W) and a minimum of Q4W.

8. A bispecific antibody which binds to human vascular endothelial growth factor (VEGF) and to human angiopoietin-2 (ANG-2), for use in the treatment of an ocular vascular disease selected from macular edema secondary to central retinal vein occlusion, secondary to hemiretinal vein occlusion or secondary to branch vein occlusion, or of patients suffering from an ocular vascular disease selected from macular edema secondary to central retinal vein occlusion, secondary to hemiretinal vein occlusion or secondary to branch vein occlusion, wherein the treatment includes a personalized treatment interval (PTI), wherein
a) patients are treated first with the bispecific VEGF/ANG2 antibody at an every 4 weeks (Q4W) dosing interval from Day 1 through Week 20
b) from Week 24, patients receive the bispecific VEGF/ANG2 antibody at a frequency of Q4W until the central subfield thickness (CST) meets a predefined reference CST threshold;
c) from this point forward, the dosing interval is extended, reduced, or maintained based on assessments made at the dosing visits which are based on the relative change of the CST and best-corrected visual acuity (BCVA) compared with the respective reference CST and BCVA;
wherein the
i) interval is extended by 4 weeks
if the CST value is increased or decreased by ≤10% without an associated ≥10-letter BCVA decrease; or
ii) interval is maintained if any of the following criteria are met:
if the CST value is decreased by >10%; or
if the CST value is decreased ≤10% with an associated ≥10-letter BCVA decrease; or
if the CST value is increased between >10% and ≤20% without an associated ≥5-letter BCVA decrease;
iii) interval is reduced by 4 weeks if any of the following criteria are met:
if the CST value is increased between >10% and ≤20% with an associated ≥5-to <10-letter BCVA decrease, or
if the CST value is increased by >20% without an associated ≥10-letter BCVA decrease, or
if the CST value is increased by <10% with an associated BCVA decrease of ≥10-letters;
iv) interval is reduced to Q4W
if the CST value is increased by >10% with an associated ≥10-letter BCVA decrease,
wherein the respective reference central subfield thickness (CST) is the CST value when the initial CST threshold criteria are met and the reference CST is adjusted if CST decreases by >10% from the previous reference CST for two consecutive dosing visits and the values obtained are within 30 μm so that the CST value obtained at the latter visit will serve as the new reference CST; and
wherein the reference best-corrected visual acuity (BCVA) is the mean of the three best BCVA scores obtained at any prior dosing visit.

9. The bispecific antibody (for use) according to claim 8, wherein the dosing interval can by adjusted to a maximum of every 16 weeks (Q16W) and a minimum of Q4W.

10. The bispecific antibody for use according to any one of claims 1 to 9, wherein the bispecific antibody which binds to human VEGF and to human ANG2 is a bispecific, bivalent anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14,
and wherein
iii) the bispecific antibody comprises a constant heavy chain region of human IgG1 subclass comprising the mutations I253A, H310A, and H435A and the mutations L234A, L235A and P329G, wherein the numberings are according to EU Index of Kabat.

11. The bispecific antibody for use according to claim 10, wherein
i) said first antigen-binding site specifically binding to VEGF comprises as heavy chain variable domain VH an amino acid sequence of SEQ ID NO: 7, and as light chain variable domain VL an amino acid sequence of SEQ ID NO: 8, and
ii) said second antigen-binding site specifically binding to ANG-2 comprises as heavy chain variable domain VH an amino acid sequence of SEQ ID NO: 15, and as light chain variable domain VL an amino acid sequence of SEQ ID NO: 16.

12. The bispecific antibody for use according to any one of claims 1 to 9, wherein the bispecific antibody which binds to human VEGF and human ANG2 comprises the amino acid sequences of SEQ ID NO: 17, of SEQ ID NO: 18, of SEQ ID NO: 19, and of SEQ ID NO: 20.

13. The bispecific antibody for use according to any one of claims 1 to 9, wherein the bispecific antibody is faricimab.

14. The bispecific antibody for use according to any one of claims 10 to 13, wherein the bispecific antibody is administered in a dose of about 5 to 7 mg.

15. The bispecific antibody for use according to any one of claims 10 to 13, wherein the bispecific antibody is administered in a dose of about 6 mg.

16. The bispecific antibody for use according to any one of claims 14 to 15, wherein the bispecific antibody is administered at a concentration of about 120 mg/ml.

17. The bispecific antibody for use according to any one of the preceding claims wherein patients suffering from an ocular vascular disease have not been previously treated with anti-VEGF treatment.

18. The bispecific antibody for use according to any one of the preceding claims wherein patients suffering from an ocular vascular disease have been previously treated with anti-VEGF treatment.

19. The bispecific antibody for use according to any one of the preceding claims wherein the antibody is administered according to determinations of a software tool.

20. A method of providing a personalized dosing schedule according to a personalized treatment interval (PTI) for the treatment of a patient suffering from nAMD, the method comprising:
receiving, at a computing system, patient data comprising a patient's CST and best-corrected visual acuity (BCVA) and optionally the information on the assessment of new macular hemorrhages; and
using the computing system, extending, reducing, or maintaining a dosing interval based on the received patient data compared with respective reference CST and BCVA; and
generating a PTI from the dosing interval, wherein the
a) interval is extended by 4 weeks (to a maximum of Q16W) if all of the following criteria are met:
i) stable CST compared with the average of the last 2 study drug dosing visits where stability is defined as a change of CST of less than 30 μm and no increase ≥50 μm in CST compared with the lowest on-study drug dosing visit measurement,
ii) no decrease ≥5 letters in BCVA compared with the average from the last two study drug dosing visits, and no decrease ≥10 letters in BCVA compared with the highest on-study drug dosing visit measurement,
iii) no new macular hemorrhage
b) interval is reduced (to a minimum Q8W) by 4 weeks if one of the following criteria is met,
or
is reduced to an 8-week interval if two or more of the following criteria are met or one criterion includes new macular hemorrhage:
i) increase of ≥50 μm in CST compared with the average from the last two dosing visits or of ≥75 μm compared with the lowest dosing visit measurement;
ii) decrease of ≥5 letters in BCVA compared with average of last two dosing visits or decrease ≥10 letters in BCVA compared with the highest dosing visit measurement;
iii) new macular hemorrhage.

21. A method of providing a personalized dosing schedule according to a personalized treatment interval (PTI) for the treatment of a patient suffering from DME, the method comprising:
receiving, at a computing system, patient data comprising a patient's CST and best-corrected visual acuity (BCVA);
using the computing system, extending, reducing, or maintaining a dosing interval based on the received patient data compared with respective reference CST and BCVA; and
generating a PTI from the dosing interval, wherein the
i) interval is extended by 4 weeks,
if the CST value is increased or decreased by ≤10% without an associated ≥10-letter BCVA decrease;
ii) interval will be maintained:
if the CST is decreased by >10%, or
the CST value is increased or decreased by ≤10% with an associated ≥10-letter BCVA decrease, or
the CST value is increased between >10% and ≤20% without an associated ≥5-letter BCVA decrease;
iii) interval is reduced by 4 weeks
if the CST value is increased between >10% and ≤20% with an associated ≥5 to <10-letter BCVA decrease; or
the CST value is increased by >20% without an associated ≥10-letter BCVA decrease;
iv) interval is reduced by 8 weeks if the CST value is increased by >10% with an associated >10-letter BCVA decrease.

22. A method of providing a personalized dosing schedule according to a personalized treatment interval (PTI) for the treatment of a patient suffering from an ocular vascular disease selected from macular edema secondary to central retinal vein occlusion, secondary to hemiretinal vein occlusion or secondary to branch vein occlusion, the method comprising:
receiving, at a computing system, patient data comprising a patient's CST and best-corrected visual acuity (BCVA);
using the computing system, extending, reducing, or maintaining a dosing interval based on the received patient data compared with respective reference CST and BCVA; and
generating a PTI from the dosing interval, wherein the
i) interval is extended by 4 weeks
if the CST value is increased or decreased by ≤10% without an associated ≥10-letter BCVA decrease; or
ii) interval is maintained if any of the following criteria are met:
if the CST value is decreased by >10%; or
if the CST value is decreased ≤10% with an associated ≥10-letter BCVA decrease; or
if the CST value is increased between >10% and ≤20% without an associated ≥5-letter BCVA decrease;
iii) interval is reduced by 4 weeks if any of the following criteria are met:
if the CST value is increased between >10% and ≤20% with an associated ≥5-to <10-letter BCVA decrease, or
if the CST value is increased by >20% without an associated ≥10-letter BCVA decrease, or
if the CST value is increased by ≤10% with an associated BCVA decrease of ≥10-letters;
iv) interval is reduced to Q4W
if the CST value is increased by >10% with an associated ≥10-letter BCVA decrease.

23. The method of any one of claim 20, 21 or 22, further comprising:
receiving, at the computing system, updated patient data;
using the computing system, continually updating or maintaining the dosing interval based on the updated patient data; and
generating a visualization, user interface, or notification based on the updated or maintained dosing interval.

24. Use of a personalized dosing schedule according to a personalized treatment interval (PTI) (for the treatment of nAMD), wherein a computing system generates the PTI by:
receiving, at a computing system, patient data comprising a patient's CST and best-corrected visual acuity (BCVA) and optionally the information on the assessment of new macular hemorrhages; and
extending, reducing, or maintaining a dosing interval based on the received patient data compared with respective reference CST and BCVA;
wherein the
a) interval is extended by 4 weeks (to a maximum of Q16W) if all of the following criteria are met:
i) stable CST compared with the average of the last 2 study drug dosing visits where stability is defined as a change of CST of less than 30 μm and no increase ≥50 μm in CST compared with the lowest on-study drug dosing visit measurement,
ii) no decrease >5 letters in BCVA compared with the average from the last two study drug dosing visits, and no decrease ≥10 letters in BCVA compared with the highest on-study drug dosing visit measurement,
iii) no new macular hemorrhage
b) interval
is reduced (to a minimum Q8W) by 4 weeks if one of the following criteria is met,
or
is reduced to an 8-week interval if two or more of the following criteria are met or one criterion includes new macular hemorrhage:
i) increase of ≥50 μm in CST compared with the average from the last two dosing visits or of ≥75 μm compared with the lowest dosing visit measurement;
ii) decrease of ≥5 letters in BCVA compared with average of last two dosing visits or decrease ≥10 letters in BCVA compared with the highest dosing visit measurement;
iii) new macular hemorrhage.

25. Use of a personalized dosing schedule according to a personalized treatment interval (PTI) (for the treatment of DME), wherein a computing system generates the PTI by:
receiving patient data comprising a patient's CST and best-corrected visual acuity (BCVA); and
extending, reducing, or maintaining a dosing interval based on the received patient data compared with respective reference CST and BCVA;
wherein the
i) interval is extended by 4 weeks,
if the CST value is increased or decreased by ≤10% without an associated ≥10-letter BCVA decrease;
ii) interval will be maintained:
if the CST is decreased by >10%, or
the CST value is increased or decreased by ≤10% with an associated ≥10-letter BCVA decrease, or
the CST value is increased between >10% and ≤20% without an associated ≥5-letter BCVA decrease;
iii) interval is reduced by 4 weeks
if the CST value is increased between >10% and ≤20% with an associated ≥5 to <10-letter BCVA decrease; or
the CST value is increased by >20% without an associated ≥10-letter BCVA decrease;
iv) interval is reduced by 8 weeks if the CST value is increased by >10% with an associated ≥10-letter BCVA decrease.

26. Use of a personalized dosing schedule according to a personalized treatment interval (PTI) (for the treatment of macular edema secondary to central retinal vein occlusion, secondary to hemiretinal vein occlusion or secondary to branch vein occlusion), wherein a computing system generates the PTI by:
receiving patient data comprising a patient's CST and best-corrected visual acuity (BCVA); and
extending, reducing, or maintaining a dosing interval based on the received patient data compared with respective reference CST and BCVA;
wherein the
i) interval is extended by 4 weeks
if the CST value is increased or decreased by ≤10% without an associated ≥10-letter BCVA decrease; or
ii) interval is maintained if any of the following criteria are met:
if the CST value is decreased by >10%; or
if the CST value is decreased ≤10% with an associated ≥10-letter BCVA decrease; or
if the CST value is increased between >10% and ≤20% without an associated ≥5-letter BCVA decrease;
iii) interval is reduced by 4 weeks if any of the following criteria are met:
if the CST value is increased between >10% and ≤20% with an associated ≥5-to <10-letter BCVA decrease, or
if the CST value is increased by >20% without an associated ≥10-letter BCVA decrease, or
if the CST value is increased by ≤10% with an associated BCVA decrease of ≥10-letters;
iv) interval is reduced to Q4W
if the CST value is increased by >10% with an associated ≥10-letter BCVA decrease.
